SEQUENCE LISTING (120 sequences)

SEQ ID NO: 1 - protein, 122 aa, Artificial Sequence
  source: /note=“Description of Artificial Sequence Synthetic polypeptide”
QVQLVQSGAEVKKPGASVKVSCKASGYIFTNYWMQWVRQAPGQGLEWMGEILPGTGSTEYAQKFQGRVTMTRDTSISTVYMEVRRLRSDDTAVYYCARYFFGSTPNWYFDVWGQGTLVTVSS

SEQ ID NO: 2 - protein, 122 aa, Artificial Sequence
  source: /note=“Description of Artificial Sequence Synthetic polypeptide”
QVQLVQSGAEVKKPGASVKVSCKASGYIFTDFYMQWVRQAPGQGLEWMGEILPGTGSTEYAQKFQGRVTMTRDTSISTVYMEVRRLRSDDTAVYYCARYFFGSTPNWYFDVWGQGTLVTVSS

SEQ ID NO: 3 - protein, 122 aa, Artificial Sequence
  source: /note=“Description of Artificial Sequence Synthetic polypeptide”
QVQLVQSGAEVKKPGASVKVSCKASGHIFTNYWMQWVRQAPGQGLEWMGEILPGTGHTEYAQKFQGRVTMTRDTSISTVYMEVRRLRSDDTAVYYCARYFFGSTPNWYFDVWGQGTLVTVSS

SEQ ID NO: 4 - protein, 122 aa, Artificial Sequence
  source: /note=“Description of Artificial Sequence Synthetic polypeptide”
QVQLVQSGAEVKKPGASVKVSCKASGHIFTDFYMQWVRQAPGQGLEWMGEILPGTGHTEYAQKFQGRVTMTRDTSISTVYMEVRRLRSDDTAVYYCARYFFGSTPNWYFDVWGQGTLVTVSS

SEQ ID NO: 5 - protein, 123 aa, Artificial Sequence
  source: /note=“Description of Artificial Sequence Synthetic polypeptide”
QVKLLEQSGAEVKKPGASVKVSCKASGYIFSNYWIQWVRQAPGQRLEWMGEILAGSGSTEYSQKFRGRVTFTRDTSATTAYMGLSSLRPEDTAVYYCARYPFGSSPNWEFDYWGQGTLVTVSS

SEQ ID NO: 6 - protein, 123 aa, Artificial Sequence
  source: /note=“Description of Artificial Sequence Synthetic polypeptide”
QVKLLEQSGAEVKKPGASVKVSCKASGFIFSNYWIQWVRQAPGQRLEWMGEVLPGSGSTEYSQKFRGRVTMTRDTSATTAYMGLSSLRPEDTAVYYCARYYFGSSPNWYFDVWGQGTLVTVSS

SEQ ID NO: 7 - protein, 123 aa, Artificial Sequence
  source: /note=“Description of Artificial Sequence Synthetic polypeptide”
QVKLLEQSGAEVKKPGASVKVSCKASGYIFTSYWIQWVRQAPGQRLEWMGEILPGDGSTEYSQKFRGRVTMTRDTSATTAYMGLSSLRPEDTAVYYCARYFFGSSPNWAMDYWGQGTLVTVSS

SEQ ID NO: 8 - protein, 123 aa, Artificial Sequence
  source: /note=“Description of Artificial Sequence Synthetic polypeptide”
QVKLLEQSGAEVKKPGASVKVSCKASGYIFTSYWIQWVRQAPGQRLEWMGEILPTNGSTEYSQKFRGRVTMTRDTSATTAYMGLSSLRPEDTAVYYCARYFFGSSPNWAMDYWGQGTLVTVSS

SEQ ID NO: 9 - protein, 116 aa, Artificial Sequence
  source: /note=“Description of Artificial Sequence Synthetic polypeptide”
QVQLVQSGAEVKKPGASVKVSCKASGYTFTGNYMHWVRQAPGQGLEYMGWINPKSGDTNYAQKFQGRVTMTRDTSISTVYMEVRRLRSDDTAVYYCATGWWGMDVWGQGTLVTVSS

SEQ ID NO: 10 - protein, 107 aa, Artificial Sequence
  source: /note=“Description of Artificial Sequence Synthetic polypeptide”
EIVMTQSPSSLSASVGDRVTITCGATENIYGALNWYQHEPGKAPKLLIYGASNLADGVTSRFSGSGSGTDFTLTISTLQPEDFATYYCQNVLNTPLSFGGGTKVDIK

SEQ ID NO: 11 - protein, 107 aa, Artificial Sequence
  source: /note=“Description of Artificial Sequence Synthetic polypeptide”
EIVMTQSPSSLSASVGDRVTITCGATENIYGALNWYQHEPGKAPKLLIYGASNRYTGVTSRFSGSGSGTDFTLTISTLQPEDFATYYCQQVLNTPVSFGGGTKVDIK

SEQ ID NO: 12 - protein, 107 aa, Artificial Sequence
  source: /note=“Description of Artificial Sequence Synthetic polypeptide”
ELVMTQSPSSLSASVGDRVNIACRASEGIYGALAWYQQKPGKAPRLLIYDASNLESGVPSRFSGSGSGTDFTLTISSLQPEDFAIYYCQQVLNTPLTFGGGTKVEIK

SEQ ID NO: 13 - protein, 107 aa, Artificial Sequence
  source: /note=“Description of Artificial Sequence Synthetic polypeptide”
ELQMTQSPSSLSASVGDRVNIACRASESIYGALNWYQQKPGKAPRLLIYDASNLESGVPSRFSGSGSGTDFTLTISSLQPEDFAIYYCQNVLSTPWTFGGGTKVEIK

SEQ ID NO: 14 - protein, 107 aa, Artificial Sequence
  source: /note=“Description of Artificial Sequence Synthetic polypeptide”
ELQMTQSPSSLSASVGDRVNIACGASENIYGALHWYQQKPGKAPRLLIYGASTRESGVPSRFSGSGSGTDFTLTISSLQPEDFAIYYCQNVLNTPYTFGGGTKVEIK

SEQ ID NO: 15 - protein, 107 aa, Artificial Sequence
  source: /note=“Description of Artificial Sequence Synthetic polypeptide”
DIVMTQSPSSLSASVGDRVTITCRASQNINNYLHWYQHEPGKAPKLLIYAASNLQGGVTSRFSGSGSGTDFTLTISTLQPEDFATYYCLQTHAYPLTFGGGTKVDIK

SEQ ID NO: 16 - protein, 7 aa, Artificial Sequence
  source: /note=“Description of Artificial Sequence Synthetic peptide”
GYIFSNY

SEQ ID NO: 17 - protein, 7 aa, Artificial Sequence
  source: /note=“Description of Artificial Sequence Synthetic peptide”
GFIFSNY

SEQ ID NO: 18 - protein, 7 aa, Artificial Sequence
  source: /note=“Description of Artificial Sequence Synthetic peptide”
GYIFTSY

SEQ ID NO: 19 - protein, 7 aa, Artificial Sequence
  source: /note=“Description of Artificial Sequence Synthetic peptide”
GYIFTNY

SEQ ID NO: 20 - protein, 8 aa, Artificial Sequence
  source: /note=“Description of Artificial Sequence Synthetic peptide”
GYIFTDFY

SEQ ID NO: 21 - protein, 7 aa, Artificial Sequence
  source: /note=“Description of Artificial Sequence Synthetic peptide”
GHIFTNY

SEQ ID NO: 22 - protein, 8 aa, Artificial Sequence
  source: /note=“Description of Artificial Sequence Synthetic peptide”
GHIFTDFY

SEQ ID NO: 23 - protein, 8 aa, Artificial Sequence
  source: /note=“Description of Artificial Sequence Synthetic peptide”
GYTFTGNY

SEQ ID NO: 24 - protein, 6 aa, Artificial Sequence
  source: /note=“Description of Artificial Sequence Synthetic peptide”
LAGSGS

SEQ ID NO: 25 - protein, 6 aa, Artificial Sequence
  source: /note=“Description of Artificial Sequence Synthetic peptide”
LPGSGS

SEQ ID NO: 26 - protein, 6 aa, Artificial Sequence
  source: /note=“Description of Artificial Sequence Synthetic peptide”
LPGDGS

SEQ ID NO: 27 - protein, 6 aa, Artificial Sequence
  source: /note=“Description of Artificial Sequence Synthetic peptide”
LPTNGS

SEQ ID NO: 28 - protein, 6 aa, Artificial Sequence
  source: /note=“Description of Artificial Sequence Synthetic peptide”
LPGTGS

SEQ ID NO: 29 - protein, 6 aa, Artificial Sequence
  source: /note=“Description of Artificial Sequence Synthetic peptide”
LPGTGH

SEQ ID NO: 30 - protein, 6 aa, Artificial Sequence
  source: /note=“Description of Artificial Sequence Synthetic peptide”
NPKSGD

SEQ ID NO: 31 - protein, 13 aa, Artificial Sequence
  source: /note=“Description of Artificial Sequence Synthetic peptide”
YPFGSSPNWEFDY

SEQ ID NO: 32 - protein, 13 aa, Artificial Sequence
  source: /note=“Description of Artificial Sequence Synthetic peptide”
YYFGSSPNWYFDV

SEQ ID NO: 33 - protein, 10 aa, Artificial Sequence
  source: /note=“Description of Artificial Sequence Synthetic peptide”
GSSPNWAMDY

SEQ ID NO: 34 - protein, 13 aa, Artificial Sequence
  source: /note=“Description of Artificial Sequence Synthetic peptide”
YFFGSSPNWAMDY

SEQ ID NO: 35 - protein, 13 aa, Artificial Sequence
  source: /note=“Description of Artificial Sequence Synthetic peptide”
YFFGSTPNWYFDV

SEQ ID NO: 36 - protein, 7 aa, Artificial Sequence
  source: /note=“Description of Artificial Sequence Synthetic peptide”
GWWGMDV

SEQ ID NO: 37 - protein, 11 aa, Artificial Sequence
  source: /note=“Description of Artificial Sequence Synthetic peptide”
RASQNINNYLH

SEQ ID NO: 38 - protein, 11 aa, Artificial Sequence
  source: /note=“Description of Artificial Sequence Synthetic peptide”
GATENIYGALN

SEQ ID NO: 39 - protein, 11 aa, Artificial Sequence
  source: /note=“Description of Artificial Sequence Synthetic peptide”
RASESIYGALN

SEQ ID NO: 40 - protein, 11 aa, Artificial Sequence
  source: /note=“Description of Artificial Sequence Synthetic peptide”
GASENIYGALH

SEQ ID NO: 41 - protein, 11 aa, Artificial Sequence
  source: /note=“Description of Artificial Sequence Synthetic peptide”
RASEGIYGALA

SEQ ID NO: 42 - protein, 7 aa, Artificial Sequence
  source: /note=“Description of Artificial Sequence Synthetic peptide”
AASNLQG

SEQ ID NO: 43 - protein, 7 aa, Artificial Sequence
  source: /note=“Description of Artificial Sequence Synthetic peptide”
GASNLAD

SEQ ID NO: 44 - protein, 7 aa, Artificial Sequence
  source: /note=“Description of Artificial Sequence Synthetic peptide”
GASNRYT

SEQ ID NO: 45 - protein, 7 aa, Artificial Sequence
  source: /note=“Description of Artificial Sequence Synthetic peptide”
DASNLES

SEQ ID NO: 46 - protein, 7 aa, Artificial Sequence
  source: /note=“Description of Artificial Sequence Synthetic peptide”
GASTRES

SEQ ID NO: 47 - protein, 9 aa, Artificial Sequence
  source: /note=“Description of Artificial Sequence Synthetic peptide”
LQTHAYPLT

SEQ ID NO: 48 - protein, 9 aa, Artificial Sequence
  source: /note=“Description of Artificial Sequence Synthetic peptide”
QNVLNTPLS

SEQ ID NO: 49 - protein, 9 aa, Artificial Sequence
  source: /note=“Description of Artificial Sequence Synthetic peptide”
QQVLNTPVS

SEQ ID NO: 50 - protein, 9 aa, Artificial Sequence
  source: /note=“Description of Artificial Sequence Synthetic peptide”
QNVLSTPWT

SEQ ID NO: 51 - protein, 9 aa, Artificial Sequence
  source: /note=“Description of Artificial Sequence Synthetic peptide”
QNVLNTPYT

SEQ ID NO: 52 - protein, 9 aa, Artificial Sequence
  source: /note=“Description of Artificial Sequence Synthetic peptide”
QQVLNTPLT

SEQ ID NO: 53 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 54 - protein, 5 aa, Artificial Sequence
  source: /note=“Description of Artificial Sequence Synthetic peptide”
NYWMQ

SEQ ID NO: 55 - protein, 5 aa, Artificial Sequence
  source: /note=“Description of Artificial Sequence Synthetic peptide”
DFYMQ

SEQ ID NO: 56 - protein, 5 aa, Artificial Sequence
  source: /note=“Description of Artificial Sequence Synthetic peptide”
NYWIQ

SEQ ID NO: 57 - protein, 5 aa, Artificial Sequence
  source: /note=“Description of Artificial Sequence Synthetic peptide”
SYWIQ

SEQ ID NO: 58 - protein, 5 aa, Artificial Sequence
  source: /note=“Description of Artificial Sequence Synthetic peptide”
GNYMH

SEQ ID NO: 59 - protein, 17 aa, Artificial Sequence
  source: /note=“Description of Artificial Sequence Synthetic peptide”
EILPGTGSTEYAQKFQG

SEQ ID NO: 60 - protein, 17 aa, Artificial Sequence
  source: /note=“Description of Artificial Sequence Synthetic peptide”
EILPGTGHTEYAQKFQG

SEQ ID NO: 61 - protein, 17 aa, Artificial Sequence
  source: /note=“Description of Artificial Sequence Synthetic peptide”
EILAGSGSTEYSQKFRG

SEQ ID NO: 62 - protein, 17 aa, Artificial Sequence
  source: /note=“Description of Artificial Sequence Synthetic peptide”
EVLPGSGSTEYSQKFRG

SEQ ID NO: 63 - protein, 17 aa, Artificial Sequence
  source: /note=“Description of Artificial Sequence Synthetic peptide”
EILPGDGSTEYSQKFRG

SEQ ID NO: 64 - protein, 17 aa, Artificial Sequence
  source: /note=“Description of Artificial Sequence Synthetic peptide”
EILPTNGSTEYSQKFRG

SEQ ID NO: 65 - protein, 17 aa, Artificial Sequence
  source: /note=“Description of Artificial Sequence Synthetic peptide”
WINPKSGDTNYAQKFQG

SEQ ID NO: 66 - protein, 13 aa, Artificial Sequence
  source: /note=“Description of Artificial Sequence Synthetic peptide”
YFFGSTPNWYFDV

SEQ ID NO: 67 - protein, 13 aa, Artificial Sequence
  source: /note=“Description of Artificial Sequence Synthetic peptide”
YPFGSSPNWEFDY

SEQ ID NO: 68 - protein, 13 aa, Artificial Sequence
  source: /note=“Description of Artificial Sequence Synthetic peptide”
YYFGSSPNWYFDV

SEQ ID NO: 69 - protein, 13 aa, Artificial Sequence
  source: /note=“Description of Artificial Sequence Synthetic peptide”
YFFGSSPNWAMDY

SEQ ID NO: 70 - protein, 7 aa, Artificial Sequence
  source: /note=“Description of Artificial Sequence Synthetic peptide”
GWWGMDV

SEQ ID NO: 71 - protein, 11 aa, Artificial Sequence
  source: /note=“Description of Artificial Sequence Synthetic peptide”
GATENIYGALN

SEQ ID NO: 72 - protein, 11 aa, Artificial Sequence
  source: /note=“Description of Artificial Sequence Synthetic peptide”
RASEGIYGALA

SEQ ID NO: 73 - protein, 11 aa, Artificial Sequence
  source: /note=“Description of Artificial Sequence Synthetic peptide”
RASESIYGALN

SEQ ID NO: 74 - protein, 11 aa, Artificial Sequence
  source: /note=“Description of Artificial Sequence Synthetic peptide”
GASENIYGALH

SEQ ID NO: 75 - protein, 11 aa, Artificial Sequence
  source: /note=“Description of Artificial Sequence Synthetic peptide”
RASQNINNYLH

SEQ ID NO: 76 - protein, 7 aa, Artificial Sequence
  source: /note=“Description of Artificial Sequence Synthetic peptide”
GASNLAD

SEQ ID NO: 77 - protein, 7 aa, Artificial Sequence
  source: /note=“Description of Artificial Sequence Synthetic peptide”
GASNRYT

SEQ ID NO: 78 - protein, 7 aa, Artificial Sequence
  source: /note=“Description of Artificial Sequence Synthetic peptide”
DASNLES

SEQ ID NO: 79 - protein, 7 aa, Artificial Sequence
  source: /note=“Description of Artificial Sequence Synthetic peptide”
GASTRES

SEQ ID NO: 80 - protein, 7 aa, Artificial Sequence
  source: /note=“Description of Artificial Sequence Synthetic peptide”
AASNLQG

SEQ ID NO: 81 - protein, 9 aa, Artificial Sequence
  source: /note=“Description of Artificial Sequence Synthetic peptide”
QNVLNTPLS

SEQ ID NO: 82 - protein, 9 aa, Artificial Sequence
  source: /note=“Description of Artificial Sequence Synthetic peptide”
QQVLNTPVS

SEQ ID NO: 83 - protein, 9 aa, Artificial Sequence
  source: /note=“Description of Artificial Sequence Synthetic peptide”
QQVLNTPLT

SEQ ID NO: 84 - protein, 9 aa, Artificial Sequence
  source: /note=“Description of Artificial Sequence Synthetic peptide”
QNVLSTPWT

SEQ ID NO: 85 - protein, 9 aa, Artificial Sequence
  source: /note=“Description of Artificial Sequence Synthetic peptide”
QNVLNTPYT

SEQ ID NO: 86 - protein, 9 aa, Artificial Sequence
  source: /note=“Description of Artificial Sequence Synthetic peptide”
LQTHAYPLT

SEQ ID NO: 87 - protein, 8 aa, Artificial Sequence
  source: /note=“Description of Artificial Sequence Synthetic peptide”
  VARIANT (2)..(2): /replace=“Phe” or “His”
  VARIANT (3)..(3): /replace=“Thr”
  VARIANT (5)..(5): /replace=“Thr”
  VARIANT (6)..(6): /replace=“Ser” or “Asp” or “Gly”
  VARIANT (7)..(7): /replace=“Asn” or “ ”
  SITE (1)..(8): /note=“Variant residues given in the sequence have no preference with respect to those in the annotations for variant positions”
  source: /note=“See specification as filed for detailed description of substitutions and preferred embodiments”
GYIFSNFY

SEQ ID NO: 88 - protein, 6 aa, Artificial Sequence
  source: /note=“Description of Artificial Sequence Synthetic peptide”
  VARIANT (1)..(1): /replace=“Asn”
  VARIANT (2)..(2): /replace=“Pro”
  VARIANT (3)..(3): /replace=“Thr” or “Lys”
  VARIANT (4)..(4): /replace=“Asp” or “Asn” or “Thr” or “Ser”
  VARIANT (6)..(6): /replace=“His” or “Asp”
  SITE (1)..(6): /note=“Variant residues given in the sequence have no preference with respect to those in the annotations for variant positions”
  source: /note=“See specification as filed for detailed description of substitutions and preferred embodiments”
LAGSGS

SEQ ID NO: 89 - protein, 13 aa, Artificial Sequence
  source: /note=“Description of Artificial Sequence Synthetic peptide”
  VARIANT (1)..(1): /replace=“Gly”
  VARIANT (2)..(2): /replace=“Tyr” or “Ser” or “Phe” or “Trp”
  VARIANT (3)..(3): /replace=“Ser” or “Trp”
  VARIANT (4)..(4): /replace=“Pro”
  VARIANT (5)..(5): /replace=“Asn” or “Met”
  VARIANT (6)..(6): /replace=“Trp” or “Thr” or “Asp”
  VARIANT (7)..(7): /replace=“Ala” or “Val”
  VARIANT (8)..(8): /replace=“Met” or “ ”
  VARIANT (9)..(9): /replace=“Asp” or “ ”
  VARIANT (10)..(10): /replace=“Tyr” or “Ala” or “ ”
  VARIANT (11)..(11): /replace=“Met” or “ ”
  VARIANT (12)..(12): /replace=“ ”
  VARIANT (13)..(13): /replace=“Val” or “ ”
  SITE (1)..(13): /note=“Variant residues given in the sequence have no preference with respect to those in the annotations for variant positions”
  source: /note=“See specification as filed for detailed description of substitutions and preferred embodiments”
YPFGSSPNWEFDY

SEQ ID NO: 90 - protein, 11 aa, Artificial Sequence
  source: /note=“Description of Artificial Sequence Synthetic peptide”
  VARIANT (1)..(1): /replace=“Gly”
  VARIANT (3)..(3): /replace=“Thr”
  VARIANT (4)..(4): /replace=“Glu”
  VARIANT (5)..(5): /replace=“Ser” or “Gly”
  VARIANT (7)..(7): /replace=“Tyr”
  VARIANT (8)..(8): /replace=“Gly”
  VARIANT (9)..(9): /replace=“Ala”
  VARIANT (11)..(11): /replace=“Asn” or “Ala”
  SITE (1)..(11): /note=“Variant residues given in the sequence have no preference with respect to those in the annotations for variant positions”
  source: /note=“See specification as filed for detailed description of substitutions and preferred embodiments”
RASQNINNYLH

SEQ ID NO: 91 - protein, 7 aa, Artificial Sequence
  source: /note=“Description of Artificial Sequence Synthetic peptide”
  VARIANT (1)..(1): /replace=“Gly” or “Asp”
  VARIANT (4)..(4): /replace=“Thr”
  VARIANT (5)..(5): /replace=“Arg”
  VARIANT (6)..(6): /replace=“Ala” or “Tyr” or “Glu”
  VARIANT (7)..(7): /replace=“Asp” or “Thr” or “Ser”
  SITE (1)..(7): /note=“Variant residues given in the sequence have no preference with respect to those in the annotations for variant positions”
  source: /note=“See specification as filed for detailed description of substitutions and preferred embodiments”
AASNLQG

SEQ ID NO: 92 - protein, 9 aa, Artificial Sequence
  source: /note=“Description of Artificial Sequence Synthetic peptide”
  VARIANT (1)..(1): /replace=“Gln”
  VARIANT (2)..(2): /replace=“Asn”
  VARIANT (3)..(3): /replace=“Val”
  VARIANT (4)..(4): /replace=“Leu”
  VARIANT (5)..(5): /replace=“Asn” or “Ser”
  VARIANT (6)..(6): /replace=“Thr”
  VARIANT (8)..(8): /replace=“Val” or “Trp” or “Tyr”
  VARIANT (9)..(9): /replace=“Ser”
  SITE (1)..(9): /note=“Variant residues given in the sequence have no preference with respect to those in the annotations for variant positions”
  source: /note=“See specification as filed for detailed description of substitutions and preferred embodiments”
LQTHAYPLT

SEQ ID NO: 93 - protein, 1685 aa, Mus musculus
MPLPAMGLWGILCLLIFLDKTWGQEQTYVISAPKILRVGSSENVVIQVHGYTEAFDATLSLKSYPDKKVTFSSGYVNLSPENKFQNAALLTLQPNQVPREESPVSHVYLEVVSKHFSKSKKIPITYNNGILFIHTDKPVYTPDQSVKIRVYSLGDDLKPAKRETVLTFIDPEGSEVDIVEENDYTGIISFPDFKIPSNPKYGVWTIKANYKKDFTTTGTAYFEIKEYVLPRFSVSIELERTFIGYKNFKNFEITVKARYFYNKVVPDAEVYAFFGLREDIKDEEKQMMHKATQAAKLVDGVAQISFDSETAVKELSYNSLEDLNNKYLYIAVTVTESSGGFSEEAEIPGVKYVLSPYTLNLVATPLFVKPGIPFSIKAQVKDSLEQAVGGVPVTLMAQTVDVNQETSDLETKRSITHDTDGVAVFVLNLPSNVTVLKFEIRTDDPELPEENQASKEYEAVAYSSLSQSYIYIAWTENYKPMLVGEYLNIMVTPKSPYIDKITHYNYLILSKGKIVQYGTREKLFSSTYQNINIPVTQNMVPSARLLVYYIVTGEQTAELVADAVWINIEEKCGNQLQVHLSPDEYVYSPGQTVSLDMVTEADSWVALSAVDRAVYKVQGNAKRAMQRVFQALDEKSDLGCGAGGGHDNADVFHLAGLTFLTNANADDSHYRDDSCKEILRSKRNLHLLRQKIEEQAAKYKHSVPKKCCYDGARVNFYETCEERVARVTIGPLCIRAFNECCTIANKIRKESPHKPVQLGRIHIKTLLPVMKADIRSYFPESWLWEIHRVPKRKQLQVTLPDSLTTWEIQGIGISDNGICVADTLKAKVFKEVFLEMNIPYSVVRGEQIQLKGTVYNYMTSGTKFCVKMSAVEGICTSGSSAASLHTSRPSRCVFQRIEGSSSHLVTFTLLPLEIGLHSINFSLETSFGKDILVKTLRVVPEGVKRESYAGVILDPKGIRGIVNRRKEFPYRIPLDLVPKTKVERILSVKGLLVGEFLSTVLSKEGINILTHLPKGSAEAELMSIAPVFYVFHYLEAGNHWNIFYPDTLSKRQSLEKKIKQGVVSVMSYRNADYSYSMWKGASASTWLTAFALRVLGQVAKYVKQDENSICNSLLWLVEKCQLENGSFKENSQYLPIKLQGTLPAEAQEKTLYLTAFSVIGIRKAVDICPTMKIHTALDKADSFLLENTLPSKSTFTLAIVAYALSLGDRTHPRFRLIVSALRKEAFVKGDPPIYRYWRDTLKRPDSSVPSSGTAGMVETTAYALLASLKLKDMNYANPIIKWLSEEQRYGGGFYSTQDTINAIEGLTEYSLLLKQIHLDMDINVAYKHEGDFHKYKVTEKHFLGRPVEVSLNDDLVVSTGYSSGLATVYVKTVVHKISVSEEFCSFYLKIDTQDIEASSHFRLSDSGFKRIIACASYKPSKEESTSGSSHAVMDISLPTGIGANEEDLRALVEGVDQLLTDYQIKDGHVILQLNSIPSRDFLCVRFRIFELFQVGFLNPATFTVYEYHRPDKQCTMIYSISDTRLQKVCEGAACTCVEADCAQLQAEVDLAISADSRKEKACKPETAYAYKVRITSATEENVFVKYTATLLVTYKTGEAADENSEVTFIKKMSCTNANLVKGKQYLIMGKEVLQIKHNFSFKYIYPLDSSTWIEYWPTDTTCPSCQAFVENLNNFAEDLFLNSCE

SEQ ID NO: 94 - protein, 5 aa, Artificial Sequence
  source: /note=“Description of Artificial Sequence Synthetic peptide”
  VARIANT (1)..(1): /replace=“Asp” or “Ser” or “Gly”
  VARIANT (2)..(2): /replace=“Phe” or “Asn”
  VARIANT (3)..(3): /replace=“Tyr”
  VARIANT (4)..(4): /replace=“Ile”
  VARIANT (5)..(5): /replace=“His”
  SITE (1)..(5): /note=“Variant residues given in the sequence have no preference with respect to those in the annotations for variant positions”
  source: /note=“See specification as filed for detailed description of substitutions and preferred embodiments”
NYWMQ

SEQ ID NO: 95 - protein, 17 aa, Artificial Sequence
  source: /note=“Description of Artificial Sequence Synthetic peptide”
  VARIANT (1)..(1): /replace=“Trp”
  VARIANT (2)..(2): /replace=“Val”
  VARIANT (3)..(3): /replace=“Asn”
  VARIANT (4)..(4): /replace=“Ala”
  VARIANT (5)..(5): /replace=“Thr” or “Lys”
  VARIANT (6)..(6): /replace=“Ser” or “Asp” or “Asn”
  VARIANT (8)..(8): /replace=“His” or “Asp”
  VARIANT (10)..(10): /replace=“Asn”
  VARIANT (12)..(12): /replace=“Ser”
  VARIANT (16)..(16): /replace=“Arg”
  SITE (1)..(17): /note=“Variant residues given in the sequence have no preference with respect to those in the annotations for variant positions”
  source: /note=“See specification as filed for detailed description of substitutions and preferred embodiments”
EILPGTGSTEYAQKFQG

SEQ ID NO: 96 - protein, 13 aa, Artificial Sequence
  source: /note=“Description of Artificial Sequence Synthetic peptide”
  VARIANT (1)..(1): /replace=“Gly”
  VARIANT (2)..(2): /replace=“Pro” or “Tyr” or “Trp”
  VARIANT (3)..(3): /replace=“ ”
  VARIANT (4)..(4): /replace=“ ”
  VARIANT (5)..(5): /replace=“ ”
  VARIANT (6)..(6): /replace=“Ser” or “ ”
  VARIANT (7)..(7): /replace=“ ”
  VARIANT (8)..(8): /replace=“ ”
  VARIANT (10)..(10): /replace=“Glu” or “Ala” or “Gly”
  VARIANT (11)..(11): /replace=“Met”
  VARIANT (13)..(13): /replace=“Tyr”
  SITE (1)..(13): /note=“Variant residues given in the sequence have no preference with respect to those in the annotations for variant positions”
  source: /note=“See specification as filed for detailed description of substitutions and preferred embodiments”
YFFGSTPNWYFDV

SEQ ID NO: 97 - protein, 11 aa, Artificial Sequence
  source: /note=“Description of Artificial Sequence Synthetic peptide”
  VARIANT (1)..(1): /replace=“Arg”
  VARIANT (3)..(3): /replace=“Ser”
  VARIANT (4)..(4): /replace=“Gln”
  VARIANT (5)..(5): /replace=“Gly” or “Ser”
  VARIANT (7)..(7): /replace=“Asn”
  VARIANT (8)..(8): /replace=“Asn”
  VARIANT (9)..(9): /replace=“Tyr”
  VARIANT (11)..(11): /replace=“Ala” or “His”
  SITE (1)..(11): /note=“Variant residues given in the sequence have no preference with respect to those in the annotations for variant positions”
  source: /note=“See specification as filed for detailed description of substitutions and preferred embodiments”
GATENIYGALN

SEQ ID NO: 98 - protein, 7 aa, Artificial Sequence
  source: /note=“Description of Artificial Sequence Synthetic peptide”
  VARIANT (1)..(1): /replace=“Asp” or “Ala”
  VARIANT (4)..(4): /replace=“Thr”
  VARIANT (5)..(5): /replace=“Arg”
  VARIANT (6)..(6): /replace=“Tyr” or “Glu” or “Gln”
  VARIANT (7)..(7): /replace=“Thr” or “Ser” or “Gly”
  SITE (1)..(7): /note=“Variant residues given in the sequence have no preference with respect to those in the annotations for variant positions”
  source: /note=“See specification as filed for detailed description of substitutions and preferred embodiments”
GASNLAD

SEQ ID NO: 99 - protein, 9 aa, Artificial Sequence
  source: /note=“Description of Artificial Sequence Synthetic peptide”
  VARIANT (1)..(1): /replace=“Leu”
  VARIANT (2)..(2): /replace=“Gln”
  VARIANT (3)..(3): /replace=“Thr”
  VARIANT (4)..(4): /replace=“His”
  VARIANT (5)..(5): /replace=“Ser” or “Ala”
  VARIANT (6)..(6): /replace=“Tyr”
  VARIANT (8)..(8): /replace=“Val” or “Trp” or “Tyr”
  VARIANT (9)..(9): /replace=“Thr”
  SITE (1)..(9): /note=“Variant residues given in the sequence have no preference with respect to those in the annotations for variant positions”
  source: /note=“See specification as filed for detailed description of substitutions and preferred embodiments”
QNVLNTPLS

SEQ ID NO: 100 - DNA, 366 bp, Artificial Sequence
  source: /note=“Description of Artificial Sequence Synthetic polynucleotide”
caggtgcagctggtgcagagcggcgcggaagtgaaaaaaccgggcgcgagcgtgaaagtgagctgcaaagcgagcggctatatttttaccaactattggatgcagtgggtgcgccaggcgccgggccagggcctggaatggatgggcgaaattctgccgggcaccggcagcaccgaatatgcgcagaaatttcagggccgcgtgaccatgacccgcgataccagcattagcaccgtgtatatggaagtgcgccgcctgcgcagcgatgataccgcggtgtattattgcgcgcgctatttttttggcagcaccccgaactggtattttgatgtgtggggccagggcaccctggtgaccgtgagcagc

SEQ ID NO: 101 - DNA, 321 bp, Artificial Sequence
  source: /note=“Description of Artificial Sequence Synthetic polynucleotide”
gaaattgtgatgacccagagcccgagcagcctgagcgcgagcgtgggcgatcgcgtgaccattacctgcggcgcgaccgaaaacatttatggcgcgctgaactggtatcagcatgaaccgggcaaagcgccgaaactgctgatttatggcgcgagcaacctggcggatggcgtgaccagccgctttagcggcagcggcagcggcaccgattttaccctgaccattagcaccctgcagccggaagattttgcgacctattattgccagaacgtgctgaacaccccgctgagctttggcggcggcaccaaagtggatattaaa

SEQ ID NO: 102 - DNA, 366 bp, Artificial Sequence
  source: /note=“Description of Artificial Sequence Synthetic polynucleotide”
caggtgcagctggtgcagagcggcgcggaagtgaaaaaaccgggcgcgagcgtgaaagtgagctgcaaagcgagcggctatatttttaccgatttttatatgcagtgggtgcgccaggcgccgggccagggcctggaatggatgggcgaaattctgccgggcaccggcagcaccgaatatgcgcagaaatttcagggccgcgtgaccatgacccgcgataccagcattagcaccgtgtatatggaagtgcgccgcctgcgcagcgatgataccgcggtgtattattgcgcgcgctatttttttggcagcaccccgaactggtattttgatgtgtggggccagggcaccctggtgaccgtgagcagc

SEQ ID NO: 103 - DNA, 321 bp, Artificial Sequence; identical to SEQ ID NO: 101
  source: /note=“Description of Artificial Sequence Synthetic polynucleotide”

SEQ ID NO: 104 - DNA, 366 bp, Artificial Sequence
  source: /note=“Description of Artificial Sequence Synthetic polynucleotide”
caggtgcagctggtgcagagcggcgcggaagtgaaaaaaccgggcgcgagcgtgaaagtgagctgcaaagcgagcggccatatttttaccaactattggatgcagtgggtgcgccaggcgccgggccagggcctggaatggatgggcgaaattctgccgggcaccggccataccgaatatgcgcagaaatttcagggccgcgtgaccatgacccgcgataccagcattagcaccgtgtatatggaagtgcgccgcctgcgcagcgatgataccgcggtgtattattgcgcgcgctatttttttggcagcaccccgaactggtattttgatgtgtggggccagggcaccctggtgaccgtgagcagc

SEQ ID NO: 105 - DNA, 321 bp, Artificial Sequence; identical to SEQ ID NO: 101
  source: /note=“Description of Artificial Sequence Synthetic polynucleotide”

SEQ ID NO: 106 - DNA, 366 bp, Artificial Sequence
  source: /note=“Description of Artificial Sequence Synthetic polynucleotide”
caggtgcagctggtgcagagcggcgcggaagtgaaaaaaccgggcgcgagcgtgaaagtgagctgcaaagcgagcggccatatttttaccgatttttatatgcagtgggtgcgccaggcgccgggccagggcctggaatggatgggcgaaattctgccgggcaccggccataccgaatatgcgcagaaatttcagggccgcgtgaccatgacccgcgataccagcattagcaccgtgtatatggaagtgcgccgcctgcgcagcgatgataccgcggtgtattattgcgcgcgctatttttttggcagcaccccgaactggtattttgatgtgtggggccagggcaccctggtgaccgtgagcagc

SEQ ID NO: 107 - DNA, 321 bp, Artificial Sequence; identical to SEQ ID NO: 101
  source: /note=“Description of Artificial Sequence Synthetic polynucleotide”

SEQ ID NO: 108 - DNA, 366 bp, Artificial Sequence; identical to SEQ ID NO: 100
  source: /note=“Description of Artificial Sequence Synthetic polynucleotide”

SEQ ID NO: 109 - DNA, 321 bp, Artificial Sequence
  source: /note=“Description of Artificial Sequence Synthetic polynucleotide”
gaaattgtgatgacccagagcccgagcagcctgagcgcgagcgtgggcgatcgcgtgaccattacctgcggcgcgaccgaaaacatttatggcgcgctgaactggtatcagcatgaaccgggcaaagcgccgaaactgctgatttatggcgcgagcaaccgctataccggcgtgaccagccgctttagcggcagcggcagcggcaccgattttaccctgaccattagcaccctgcagccggaagattttgcgacctattattgccagcaggtgctgaacaccccggtgagctttggcggcggcaccaaagtggatattaaa

SEQ ID NO: 110 - DNA, 366 bp, Artificial Sequence; identical to SEQ ID NO: 102
  source: /note=“Description of Artificial Sequence Synthetic polynucleotide”

SEQ ID NO: 111 - DNA, 321 bp, Artificial Sequence; identical to SEQ ID NO: 109
  source: /note=“Description of Artificial Sequence Synthetic polynucleotide”

SEQ ID NO: 112 - DNA, 366 bp, Artificial Sequence; identical to SEQ ID NO: 104
  source: /note=“Description of Artificial Sequence Synthetic polynucleotide”

SEQ ID NO: 113 - DNA, 321 bp, Artificial Sequence; identical to SEQ ID NO: 109
  source: /note=“Description of Artificial Sequence Synthetic polynucleotide”

SEQ ID NO: 114 - DNA, 366 bp, Artificial Sequence; identical to SEQ ID NO: 106
  source: /note=“Description of Artificial Sequence Synthetic polynucleotide”

SEQ ID NO: 115 - DNA, 321 bp, Artificial Sequence; identical to SEQ ID NO: 109
  source: /note=“Description of Artificial Sequence Synthetic polynucleotide”

SEQ ID NO: 116 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 119 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 107 aa, Artificial Sequence [sequence omitted]

(19) United States
(12) Patent Application Publication
(10) Pub. No.: US 2021/0238268 A1
(43) Pub. Date: Aug. 5, 2021

(54) ANTIBODY MOLECULES TO COMPLEMENT COMPONENT 5 AND USES THEREOF

(71) Applicant: Atarga, LLC, Winchester, MA (US)

(72) Inventors: Akila Jayaraman, Winchester, MA (US); Akshay Paintal, Winchester, MA (US)

(21) Appl. No.: 17/253,569

(22) PCT Filed: Jun. 19, 2019
(86) PCT No.: PCT/US2019/038027

§ 371 Date: Dec. 17, 2020

Related U.S. Application Data
(60) Provisional application No. 62/686,982, filed on Jun. 19, 2018.

(51) Int. Cl. C07K 16/18; G01N 33/577

(52) U.S. Cl. CPC C07K 16/18; G01N 33/577; G01N 2333/4716; C07K 2317/31; C07K 2317/92; C07K 2317/24

(57) ABSTRACT

Antibody molecules that specifically bind to C5 are disclosed. The antibody molecules can be used to treat, prevent, and/or diagnose disorders, such as complement-associated disorders.

### CROSS REFERENCE TO RELATED APPLICATIONS

This application claims the benefit of U.S. Provisional Application No. 62/686,982, filed Jun. 19, 2018. The contents of the aforementioned application are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 19, 2019, is named A2176-7000WO_SL and is 101,767 bytes in size.

### BACKGROUND

Complement proteins are part of the innate immune response. They perform a range of biological functions such as opsonization (coating foreign pathogens), initiating the membrane attack complex, and enhancing inflammation, by activating different pathways: classical, lectin, and alternate. Complement pathways converge to a common pathway that causes splitting or activation of C3 to make C3a or C3b, resulting in the formation of various bioactive molecules such as C5a and C5b. Experimental studies have shown that blockade of C5 cleavage into C5a and C5b can have anti-inflammatory effects without affecting the activation and function of the early components (e.g. Rother R P et. al., Nature Biotechnology 2007; 25(11): 1256-1264, Fukuzawa T et. al., Sci Rep. 2017; 7(1): 1080; each of which is incorporated herein by reference in its entirety). There is a need for developing new approaches for treating, preventing and diagnosing complement-associated disorders and other disorders that share similar disease mechanisms.

### SUMMARY

This disclosure provides, at least in part, antibody molecules that bind to Complement component 5 (C5), e.g., human C5 (e.g., human C5 comprising the amino acid sequence of SEQ ID NO: 53), and that comprise one or more functional and structural properties disclosed herein. In an embodiment, the antibody molecule binds to and/or reduces (e.g., inhibits, blocks, or neutralizes) one or more activities of C5. In an embodiment, the antibody molecule is selected from Table 1, or competes for binding to C5 with an antibody molecule selected from Table 1. In an embodiment, the antibody molecule binds to the same or overlapping epitope as the epitope recognized by an antibody molecule selected from Table 1. In an embodiment, the antibody molecule comprises one or more heavy chain variable regions and/or one or more light chain variable regions described in Table 1. In an embodiment, the antibody molecule comprises one or more heavy chain CDRs and/or one or more light chain CDRs described in Table 1. In an embodiment, nucleic acid molecules encoding the antibody molecules, expression vectors, host cells, compositions (e.g., pharmaceutical compositions), kits, containers, and methods for making the antibody molecules, are also provided. The antibody molecules disclosed herein are suitable for use in reducing or inhibiting undesired activation of the complement system or a component thereof. The antibody molecules disclosed herein can be used (alone or in combination with other agents or therapeutic modalities) to treat, prevent and/or diagnose complement-associated disorders.

Accordingly, in an aspect, this disclosure provides an antibody molecule, e.g., an antibody molecule described herein, having one or more (e.g., 1, 2, 3, 4, 5, 6, 7, 8, 9, 10, 11, 12, 13, 14, 15, or all) of the following properties:
a) Binds to C5 (e.g., human C5) with high affinity, e.g., with a dissociation constant (K_{D}′) of about 50 nM or less, e.g., about 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.5 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 10 nM, between 0.001 nM and 5 nM, between 0.001 nM and 2 nM, between 0.001 nM and 1 nM, between 0.001 nM and 0.5 nM, between 0.001 nM and 0.2 nM, between 0.001 nM and 0.1 nM, between 0.001 and 0.05 nM, between 0.001 and 0.02 nM, between 0.001 and 0.005 nM, between 5 nM and 10 nM, between 2 nM and 10 nM, between 1 nM and 10 nM, between 0.5 nM and 10 nM, between 0.2 nM and 10 nM, between 0.1 nM and 10 nM, between 0.05 nM and 10 nM, between 0.02 nM and 10 nM, between 0.01 nM and 10 nM, between 0.005 nM and 10 nM, between 0.002 and 10 nM, between 0.002 nM and 5 nM, between 0.005 nM and 2 nM, between 0.01 nM and 1 nM, between 0.02 nM and 0.5 nM, between 0.05 nM and 0.2 nM, between 0.001 nM and 0.002 nM, between 0.002 nM and 0.005 nM, between 0.005 nM and 0.01 nM, between 0.01 nM and 0.02 nM, between 0.02 nM and 0.05 nM, between 0.05 nM and 0.1 nM, between 0.1 nM and 0.2 nM, between 0.2 nM and 0.5 nM, between 0.5 nM and 1 nM, between 1 nM and 2 nM, between 2 nM and 5 nM, or between 5 nM and 10 nM, e.g., between 0.1 nM and 0.6 nM or between 0.2 nM and 0.53 nM, e.g., as determined by a method described herein;
b) Binds to C5 (e.g., human C5) at the neutral pH with an affinity that is at least 1, 2, 3, 4, 5, 6, 7, 8, 9, 10-fold higher than the affinity at an acidic pH, e.g., a pH below 7, 6.5, 6, 5.5, 5, or lower;
c) Binds to C5 (e.g., human C5) with high affinity, e.g., with a half maximal effective concentration (EC₅₀) of about 2 μg/ml or less, e.g., about 1 μg/ml or less, 0.9 μg/ml or less, 0.8 μg/ml or less, 0.7 μg/ml or less, 0.6 μg/ml or less, 0.5 μg/ml or less, 0.4 μg/ml or less, 0.3 μg/ml or less, 0.2 μg/ml or less, 0.1 μg/ml or less, 0.09 μg/ml or less, 0.08 μg/ml or less, 0.07 μg/ml or less, 0.06 μg/ml or less, 0.05 μg/ml or less, 0.04 μg/ml or less, 0.03 μg/ml or less, 0.02 μg/ml or less, 0.01 μg/ml or less, 0.005 μg/ml or less, 0.002 μg/ml or less, 0.001 μg/ml or less, e.g., between 0.001 μg/ml and 2 μg/ml, e.g., between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 μg/ml and 0.05 μg/ml, between 0.001 μg/ml and 0.02 μg/ml, between 0.001 μg/ml and 0.01 μg/ml, between 0.001 μg/ml and 0.005 μg/ml, between 0.002 μg/ml and 1 μg/ml, between 0.005 μg/ml and 1 μg/ml, between 0.01 μg/ml and 1 μg/ml, between 0.02 μg/ml and 1 μg/ml, between 0.05 μg/ml and 1 μg/ml, between 0.1 μg/ml and 1 μg/ml, between 0.2 μg/ml and 1 μg/ml, between 0.5 μg/ml and 1 μg/ml, between 0.001 μg/ml and 1 μg/ml, between 0.002 μg/ml and 0.5 μg/ml, between 0.005 μg/ml and 0.2 μg/ml, between 0.01 μg/ml and 0.1 μg/ml, or between 0.02 μg/ml and 0.05 μg/ml, e.g., as determined by a method described herein;
d) Binds specifically to an epitope on C5 (e.g., human C5), e.g., the same, similar, or overlapping epitope as the epitope recognized by a monoclonal antibody described in Table 1, e.g., any of monoclonal antibodies ATG-001, ATG-002, ATG-003, ATG-004, ATG-005, ATG-006, ATG-007, ATG-008, ATG-012, or ATG-013;
e) Reduces (e.g., inhibits, blocks, or neutralizes) one or more biological activities of C5 (e.g., human C5), in vitro, ex vivo, or in vivo;
f) Reduces (e.g., inhibits, blocks, or neutralizes) one or more biological activities of C5 (e.g., human C5), e.g., at a half maximal inhibitory concentration (IC₅₀) of about 50 μg/ml or less, e.g., about 20 μg/ml or less, 10 μg/ml or less, 9 μg/ml or less, 8 μg/ml or less, 7 μg/ml or less, 6 μg/ml or less, 5 μg/ml or less, 4 μg/ml or less, 3 μg/ml or less, 2 μg/ml or less, 1 μg/ml or less, 0.5 μg/ml or less, 0.2 μg/ml or less, 0.1 μg/ml or less, 0.05 μg/ml or less, 0.02 μg/ml or less, 0.01 μg/ml or less, 0.005 μg/ml or less, 0.002 μg/ml or less, or 0.001 μg/ml or less, e.g., between 0.001 μg/ml and 10 μg/ml, between 0.001 μg/ml and 5 μg/ml, between 0.001 μg/ml and 2 μg/ml, between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 and 0.05 μg/ml, between 0.001 and 0.02 μg/ml, between 0.001 and 0.005 μg/ml, between 5 μg/ml and 10 μg/ml, between 2 μg/ml and 10 μg/ml, between 1 μg/ml and 10 μg/ml, between 0.5 μg/ml and 10 μg/ml, between 0.2 μg/ml and 10 μg/ml, between 0.1 μg/ml and 10 μg/ml, between 0.05 μg/ml and 10 μg/ml, between 0.02 μg/ml and 10 μg/ml, between 0.01 μg/ml and 10 μg/ml, between 0.005 μg/ml and 10 μg/ml, between 0.002 and 10 μg/ml, between 0.002 μg/ml and 5 μg/ml, between 0.005 μg/ml and 2 μg/ml, between 0.01 μg/ml and 1 μg/ml, between 0.02 μg/ml and 0.5 μg/ml, between 0.05 μg/ml and 0.2 μg/ml, between 0.001 μg/ml and 0.002 μg/ml, between 0.002 μg/ml and 0.005 μg/ml, between 0.005 μg/ml and 0.01 μg/ml, between 0.01 μg/ml and 0.02 μg/ml, between 0.02 μg/ml and 0.05 μg/ml, between 0.05 μg/ml and 0.1 μg/ml, between 0.1 μg/ml and 0.2 μg/ml, between 0.2 μg/ml and 0.5 μg/ml, between 0.5 μg/ml and 1 μg/ml, between 1 μg/ml and 2 μg/ml, between 2 μg/ml and 5 μg/ml, or between 5 μg/ml and 10 μg/ml, e.g., between 1 μg/ml and 8 μg/ml or between 2 μg/ml and 6 μg/ml, e.g., as determined by a method described herein;
g) Shows the same or similar binding affinity or specificity, or both, as a monoclonal antibody described in Table 1, e.g., any of monoclonal antibodies ATG-001, ATG-002, ATG-003, ATG-004, ATG-005, ATG-006, ATG-007, ATG-008, ATG-012, or ATG-013;
h) Shows the same or similar binding affinity or specificity, or both, as an antibody molecule comprising a heavy chain variable region and/or light chain variable region described in Table 1, e.g., a heavy chain variable region and/or light chain variable region of any of monoclonal antibodies ATG-001, ATG-002, ATG-003, ATG-004, ATG-005, ATG-006, ATG-007, ATG-008, ATG-012, or ATG-013;
i) Shows the same or similar binding affinity or specificity, or both, as an antibody molecule comprising one or more (e.g., two or three) heavy chain CDRs and/or one or more (e.g., two or three) light chain CDRs described in Table 1, e.g., one or more (e.g., two or three) heavy chain CDRs and/or one or more (two or three) light chain CDRs of any of monoclonal antibodies ATG-001, ATG-002, ATG-003, ATG-004, ATG-005, ATG-006, ATG-007, ATG-008, ATG-012, or ATG-013;
j) Shows the same or similar binding affinity or specificity, or both, as an antibody molecule comprising an amino acid sequence shown in Table 1;
k) Shows the same or similar binding affinity or specificity, or both, as an antibody molecule comprising an amino acid sequence encoded by a nucleotide sequence shown in Table 5;
l) Inhibits, e.g., competitively inhibits, the binding of a second antibody molecule to C5 (e.g., human C5), e.g., human C5, wherein the second antibody molecule is an antibody molecule chosen from Table 1, e.g., any of monoclonal antibodies ATG-001, ATG-002, ATG-003, ATG-004, ATG-005, ATG-006, ATG-007, ATG-008, ATG-012, or ATG-013;
m) Competes for binding with a second antibody molecule to C5 (e.g., human C5), wherein the second antibody molecule is a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013;
n) Has one or more biological properties of a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013;
o) Has one or more structural properties of a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013; or
p) Has one or more pharmacokinetic properties of a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule binds to C5 (e.g., human C5) with high affinity, e.g., with a K_{D}′ of about 50 nM or less, e.g., about 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.5 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 10 nM, between 0.001 nM and 5 nM, between 0.001 nM and 2 nM, between 0.001 nM and 1 nM, between 0.001 nM and 0.5 nM, between 0.001 nM and 0.2 nM, between 0.001 nM and 0.1 nM, between 0.001 and 0.05 nM, between 0.001 and 0.02 nM, between 0.001 and 0.005 nM, between 5 nM and 10 nM, between 2 nM and 10 nM, between 1 nM and 10 nM, between 0.5 nM and 10 nM, between 0.2 nM and 10 nM, between 0.1 nM and 10 nM, between 0.05 nM and 10 nM, between 0.02 nM and 10 nM, between 0.01 nM and 10 nM, between 0.005 nM and 10 nM, between 0.002 and 10 nM, between 0.002 nM and 5 nM, between 0.005 nM and 2 nM, between 0.01 nM and 1 nM, between 0.02 nM and 0.5 nM, between 0.05 nM and 0.2 nM, between 0.001 nM and 0.002 nM, between 0.002 nM and 0.005 nM, between 0.005 nM and 0.01 nM, between 0.01 nM and 0.02 nM, between 0.02 nM and 0.05 nM, between 0.05 nM and 0.1 nM, between 0.1 nM and 0.2 nM, between 0.2 nM and 0.5 nM, between 0.5 nM and 1 nM, between 1 nM and 2 nM, between 2 nM and 5 nM, or between 5 nM and 10 nM, e.g., between 0.1 nM and 0.6 nM or between 0.2 nM and 0.53 nM, e.g., as determined by a method described herein.

In an embodiment, the antibody molecule binds to C5 (e.g., human C5) at the neutral pH with an affinity that is at least 1, 2, 3, 4, 5, 6, 7, 8, 9, 10-fold higher than the affinity at an acidic pH, e.g., a pH below 7, 6.5, 6, 5.5, 5, or lower.

In an embodiment, the antibody molecule binds to C5 (e.g., human C5) with high affinity, e.g., with an EC₅₀ of about 2 μg/ml or less, e.g., about 1 μg/ml or less, 0.9 μg/ml or less, 0.8 μg/ml or less, 0.7 μg/ml or less, 0.6 μg/ml or less, 0.5 μg/ml or less, 0.4 μg/ml or less, 0.3 μg/ml or less, 0.2 μg/ml or less, 0.1 μg/ml or less, 0.09 μg/ml or less, 0.08 μg/ml or less, 0.07 μg/ml or less, 0.06 μg/ml or less, 0.05 μg/ml or less, 0.04 μg/ml or less, 0.03 μg/ml or less, 0.02 μg/ml or less, 0.01 μg/ml or less, 0.005 μg/ml or less, 0.002 μg/ml or less, 0.001 μg/ml or less, e.g., between 0.001 μg/ml and 2 μg/ml, e.g., between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 μg/ml and 0.05 μg/ml, between 0.001 μg/ml and 0.02 μg/ml, between 0.001 μg/ml and 0.01 μg/ml, between 0.001 μg/ml and 0.005 μg/ml, between 0.002 μg/ml and 1 μg/ml, between 0.005 μg/ml and 1 μg/ml, between 0.01 μg/ml and 1 μg/ml, between 0.02 μg/ml and 1 μg/ml, between 0.05 μg/ml and 1 μg/ml, between 0.1 μg/ml and 1 μg/ml, between 0.2 μg/ml and 1 μg/ml, between 0.5 μg/ml and 1 μg/ml, between 0.001 μg/ml and 1 μg/ml, between 0.002 μg/ml and 0.5 μg/ml, between 0.005 μg/ml and 0.2 μg/ml, between 0.01 μg/ml and 0.1 μg/ml, or between 0.02 μg/ml and 0.05 μg/ml, e.g., as determined by a method described herein.

In an embodiment, the antibody molecule binds specifically to an epitope on C5 (e.g., human C5), e.g., the same, similar, or overlapping epitope as the epitope recognized by a monoclonal antibody described in Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule reduces (e.g., inhibits, blocks, or neutralizes) one or more biological activities of C5 (e.g., human C5), in vitro, ex vivo, or in vivo.

In an embodiment, the antibody molecule reduces (e.g., inhibits, blocks, or neutralizes) one or more biological activities of C5 (e.g., human C5), e.g., at an IC₅₀ of about 50 μg/ml or less, e.g., about 20 μg/ml or less, 10 μg/ml or less, 9 μg/ml or less, 8 μg/ml or less, 7 μg/ml or less, 6 μg/ml or less, 5 μg/ml or less, 4 μg/ml or less, 3 μg/ml or less, 2 μg/ml or less, 1 μg/ml or less, 0.5 μg/ml or less, 0.2 μg/ml or less, 0.1 μg/ml or less, 0.05 μg/ml or less, 0.02 μg/ml or less, 0.01 μg/ml or less, 0.005 μg/ml or less, 0.002 μg/ml or less, or 0.001 μg/ml or less, e.g., between 0.001 μg/ml and 10 μg/ml, between 0.001 μg/ml and 5 μg/ml, between 0.001 μg/ml and 2 μg/ml, between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 and 0.05 μg/ml, between 0.001 and 0.02 μg/ml, between 0.001 and 0.005 μg/ml, between 5 μg/ml and 10 μg/ml, between 2 μg/ml and 10 μg/ml, between 1 μg/ml and 10 μg/ml, between 0.5 μg/ml and 10 μg/ml, between 0.2 μg/ml and 10 μg/ml, between 0.1 μg/ml and 10 μg/ml, between 0.05 μg/ml and 10 μg/ml, between 0.02 μg/ml and 10 μg/ml, between 0.01 μg/ml and 10 μg/ml, between 0.005 μg/ml and 10 μg/ml, between 0.002 and 10 μg/ml, between 0.002 μg/ml and 5 μg/ml, between 0.005 μg/ml and 2 μg/ml, between 0.01 μg/ml and 1 μg/ml, between 0.02 μg/ml and 0.5 μg/ml, between 0.05 μg/ml and 0.2 μg/ml, between 0.001 μg/ml and 0.002 μg/ml, between 0.002 μg/ml and 0.005 μg/ml, between 0.005 μg/ml and 0.01 μg/ml, between 0.01 μg/ml and 0.02 μg/ml, between 0.02 μg/ml and 0.05 μg/ml, between 0.05 μg/ml and 0.1 μg/ml, between 0.1 μg/ml and 0.2 μg/ml, between 0.2 μg/ml and 0.5 μg/ml, between 0.5 μg/ml and 1 μg/ml, between 1 μg/ml and 2 μg/ml, between 2 μg/ml and 5 μg/ml, or between 5 μg/ml and 10 μg/ml, e.g., between 1 μg/ml and 8 μg/ml or between 2 μg/ml and 6 μg/ml, e.g., as determined by a method described herein.

In an embodiment, the antibody molecule shows the same or similar binding affinity or specificity, or both, as a monoclonal antibody described in Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule shows the same or similar binding affinity or specificity, or both, as an antibody molecule comprising a heavy chain variable region and/or light chain variable region described in Table 1, e.g., a heavy chain variable region and/or light chain variable region of any of monoclonal antibodies ATG-001, ATG-002, ATG-003, ATG-004, ATG-005, ATG-006, ATG-007, ATG-008, ATG-012, or ATG-013.

In an embodiment, the antibody molecule shows the same or similar binding affinity or specificity, or both, as an antibody molecule comprising one or more (e.g., two or three) heavy chain CDRs and/or one or more (e.g., two or three) light chain CDRs described in Table 1, e.g., one or more (e.g., two or three) heavy chain CDRs and/or one or more (two or three) light chain CDRs of any of monoclonal antibodies ATG-001, ATG-002, ATG-003, ATG-004, ATG-005, ATG-006, ATG-007, ATG-008, ATG-012, or ATG-013.

In an embodiment, the antibody molecule shows the same or similar binding affinity or specificity, or both, as an antibody molecule comprising an amino acid sequence shown in Table 1,

In an embodiment, the antibody molecule shows the same or similar binding affinity or specificity, or both, as an antibody molecule comprising an amino acid sequence encoded by a nucleotide sequence shown in Table 5.

In an embodiment, the antibody molecule inhibits, e.g., competitively inhibits, the binding of a second antibody molecule to C5 (e.g., human C5) wherein the second antibody molecule is an antibody molecule chosen from Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule competes for binding with a second antibody molecule to C5 (e.g., human C5), wherein the second antibody molecule is a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule has one or more biological properties of a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule has one or more structural properties of a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule has one or more pharmacokinetic properties of a monoclonal antibody chosen from Table 1, e.g., any of monoclonal antibodies ATG-001, ATG-002, ATG-003, ATG-004, ATG-005, ATG-006, ATG-007, ATG-008, ATG-012, or ATG-013.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In some embodiments, the antibody molecule comprises a heavy chain variable region (VH) and a light chain variable region (VL), wherein the VH comprises three heavy chain complementarity determining regions (HCDR1, HCDR2, and HCDR3), wherein the VL comprises three light chain complementarity determining regions (LCDR1, LCDR2, and LCDR3), wherein the VH comprises one, two, or all of the following:

(i) an HCDR1 comprising the amino acid sequence:

GX₁X₂FX₃X₄X₅Y,
wherein: X₁ is Y, F, or H;
   X₂ is I or T;
   X₃ is S or T;
   X₄ is N, S, D, or G; and
   X₅ is F, N, or absent
(SEQ ID NO: 87);

(ii) an HCDR2 comprising the amino acid sequence:

X₁X₂X₃X₄GX₅,
wherein: X₁ is L or N;
   X₂ is A or P;
   X₃ is G, T, or K;
   X₄ is S, D, N, T, or S; and
   X₅ is S, H, or D
(SEQ ID NO: 88);

(iii) an HCDR3 comprising the amino acid sequence:

X₁X₂X₃X₄X₅X₆X₇X₈X₉X₁₀X₁₁X₁₂X₁₃,
wherein: X₁ is Y or G;
   X₂ is P, Y, S, F, or W;
   X₃ is F, S, or W;
   X₄ is G or P;
   X₅ is S, N, or M;
   X₆ is S, W, T, or D;
   X₇ is P, A, or V;
   X₈ is N, M, or absent;
   X₉ is W, D, or absent;
   X₁₀ is E, Y, A, or absent;
   X₁₁ is F, M, or absent;
   X₁₂ is D or absent; and
   X₁₃ is Y, V, or absent
(SEQ ID NO: 89); and

wherein the VL comprises one, two, or all of the following:

(iv) an LCDR1 comprising the amino acid sequence:

X₁AX₂X₃X₄IX₅X₆X₇LX₈,
wherein: X₁ is R or G;
   X₂ is S or T;
   X₃ is Q or E;
   X₄ is N, S, or G;
   X₅ is N or Y;
   X₆ is N or G;
   X₇ is Y or A; and
   X₈ is H, N, or A
(SEQ ID NO: 90);

(v) an LCDR2 comprising the amino acid sequence:

X₁ASX₂X₃X₄X₅,
wherein: X₁ is A, G, or D;
   X₂ is N or T;
   X₃ is L or R;
   X₄ is Q, A, Y, or E; or
   X₅ is G, D, T, or S
(SEQ ID NO: 91); and

(vi) an LCDR3 comprising the amino acid sequence:

X₁X₂X₃X₄X₅X₆PX₇X₈,
wherein: X₁ is L or Q;
   X₂ is Q or N;
   X₃ is T or V;
   X₄ is H or L;
   X₅ is A, N, or S;
   X₆ is Y or T;
   X₇ is L, V, W, or Y; or
   X₈ is T or S
(SEQ ID NO: 92).

In embodiments, the antibody molecule comprises a VH and a VL, wherein the VH comprises three heavy chain complementarity determining regions (HCDR1, HCDR2, and HCDR3), wherein the VL comprises three light chain complementarity determining regions (LCDR1, LCDR2, and LCDR3),

wherein the VH comprises one, two, or all of the following:

(i) an HCDR1 comprising the amino acid sequence:

X₁X₂X₃X₄X₅
wherein: X₁ is N, D, S, or G;
   X₂ is Y, F, or N;
   X₃ is W or Y;
   X₄ is M or I; and
   X₅ is Q or H
(SEQ ID NO: 94);

(ii) an HCDR2 comprising the amino acid sequence:

X₁X₂X₃X₄X₅X₆GX₇TX₈YX₉QKFX₁₀G
wherein: X₁ is E or W;
   X₂ is I or V;
   X₃ is L or N;
   X₄ is P or A;
   X₅ is G, T, or K;
   X₆ is T, S, D, or N;
   X₇ is S, H, or D;
   X₈ is E or N;
   X₉ is A or S; and
   X₁₀ is Q or R
(SEQ ID NO: 95);

(iii) an HCDR3 comprising the amino acid sequence:

X₁X₂X₃X₄X₅X₆X₇X₈WX₉X₁₀DX₁₁
wherein: X₁ is Y or G;
   X₂ is F, P, Y, or W;
   X₃ is F or absent;
   X₄ is G or absent;
   X₅ is S or absent;
   X₆ is T, S, or absent;
   X₇ is P or absent;
   X₈ is N or absent;
   X₉ is Y, E, A, or G;
   X₁₀ is F or M; and
   X₁₁ is V or Y
(SEQ ID NO: 96); and

wherein the VL comprises one, two, or all of the following:

(iv) an LCDR1 comprising the amino acid sequence:

X₁AX₂X₃X₄IX₅X₆X₇LX₈
wherein: X₁ is G or R;
   X₂ is T or S;
   X₃ is E or Q;
   X₄ is N, G, or S;
   X₅ is Y or N;
   X₆ is G or N;
   X₇ is A or Y; and
   X₈ is N, A, or H
(SEQ ID NO: 97);

(v) an LCDR2 comprising the amino acid sequence:

X₁ASX₂X₃X₄X₅
wherein: X₁ is G, D, or A;
   X₂ is N or T;
   X₃ is L or R;
   X₄ is A, Y, E, or Q; and
   X₅ is D, T, S, or G
(SEQ ID NO: 98); and

(vi) an LCDR3 comprising the amino acid sequence:

X₁X₂X₃X₄X₅X₆PX₇X₈
wherein: X₁ is Q or L;
   X₂ is N or Q;
   X₃ is V or T;
   X₄ is L or H;
   X₅ is N, S, or A;
   X₆ is Tor Y;
   X₇ is L, V, W, or Y; and
   X₈ is S or T

(SEQ ID NO: 99).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-001 (e.g., SEQ ID NO: 19); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-001 (e.g., SEQ ID NO: 28); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-001 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-001 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-001 (e.g., SEQ ID NO: 43); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-001 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-001 (e.g., SEQ ID NO: 19); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-001 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-001 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-001 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-001 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-001 (e.g., SEQ ID NO: 48).

In an embodiment, the antibody molecule comprises: (i) a VH comprising: an HCDR1 comprising the amino acid sequence of the HCDR1 of monoclonal antibody ATG-001 (e.g., SEQ ID NO: 19); an HCDR2 comprising the amino acid sequence of the HCDR2 of monoclonal antibody ATG-001 (e.g., SEQ ID NO: 28); and an HCDR3 comprising the amino acid sequence of the HCDR3 of monoclonal antibody ATG-001 (e.g., SEQ ID NO: 35), and (ii) a VL comprising: an LCDR1 comprising the amino acid sequence of the LCDR1 of monoclonal antibody ATG-001 (e.g., SEQ ID NO: 38); an LCDR2 comprising the amino acid sequence of the LCDR2 of monoclonal antibody ATG-001 (e.g., SEQ ID NO: 43); and an LCDR3 comprising the amino acid sequence of the LCDR3 of monoclonal antibody ATG-001 (e.g., SEQ ID NO: 48).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-002 (e.g., SEQ ID NO: 20); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-002 (e.g., SEQ ID NO: 28); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-002 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-002 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-002 (e.g., SEQ ID NO: 43); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-002 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-002 (e.g., SEQ ID NO: 20); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-002 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-002 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-002 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-002 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-002 (e.g., SEQ ID NO: 48).

In an embodiment, the antibody molecule comprises: (i) a VH comprising: an HCDR1 comprising the amino acid sequence of the HCDR1 of monoclonal antibody ATG-002 (e.g., SEQ ID NO: 20); an HCDR2 comprising the amino acid sequence of the HCDR2 of monoclonal antibody ATG-002 (e.g., SEQ ID NO: 28); and an HCDR3 comprising the amino acid sequence of the HCDR3 of monoclonal antibody ATG-002 (e.g., SEQ ID NO: 35), and (ii) a VL comprising: an LCDR1 comprising the amino acid sequence of the LCDR1 of monoclonal antibody ATG-002 (e.g., SEQ ID NO: 38); an LCDR2 comprising the amino acid sequence of the LCDR2 of monoclonal antibody ATG-002 (e.g., SEQ ID NO: 43); and an LCDR3 comprising the amino acid sequence of the LCDR3 of monoclonal antibody ATG-002 (e.g., SEQ ID NO: 48).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-003 (e.g., SEQ ID NO: 21); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-003 (e.g., SEQ ID NO: 29); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-003 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-003 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-003 (e.g., SEQ ID NO: 43); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-003 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-003 (e.g., SEQ ID NO: 21); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-003 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-003 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-003 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-003 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-003 (e.g., SEQ ID NO: 48).

In an embodiment, the antibody molecule comprises: (i) a VH comprising: an HCDR1 comprising the amino acid sequence of the HCDR1 of monoclonal antibody ATG-003 (e.g., SEQ ID NO: 21); an HCDR2 comprising the amino acid sequence of the HCDR2 of monoclonal antibody ATG-003 (e.g., SEQ ID NO: 29); and an HCDR3 comprising the amino acid sequence of the HCDR3 of monoclonal antibody ATG-003 (e.g., SEQ ID NO: 35), and (ii) a VL comprising: an LCDR1 comprising the amino acid sequence of the LCDR1 of monoclonal antibody ATG-003 (e.g., SEQ ID NO: 38); an LCDR2 comprising the amino acid sequence of the LCDR2 of monoclonal antibody ATG-003 (e.g., SEQ ID NO: 43); and an LCDR3 comprising the amino acid sequence of the LCDR3 of monoclonal antibody ATG-003 (e.g., SEQ ID NO: 48).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-004 (e.g., SEQ ID NO: 22); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-004 (e.g., SEQ ID NO: 29); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-004 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-004 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-004 (e.g., SEQ ID NO: 43); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-004 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-004 (e.g., SEQ ID NO: 22); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-004 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-004 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-004 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-004 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-004 (e.g., SEQ ID NO: 48).

In an embodiment, the antibody molecule comprises: (i) a VH comprising: an HCDR1 comprising the amino acid sequence of the HCDR1 of monoclonal antibody ATG-004 (e.g., SEQ ID NO: 22); an HCDR2 comprising the amino acid sequence of the HCDR2 of monoclonal antibody ATG-004 (e.g., SEQ ID NO: 29); and an HCDR3 comprising the amino acid sequence of the HCDR3 of monoclonal antibody ATG-004 (e.g., SEQ ID NO: 35), and (ii) a VL comprising: an LCDR1 comprising the amino acid sequence of the LCDR1 of monoclonal antibody ATG-004 (e.g., SEQ ID NO: 38); an LCDR2 comprising the amino acid sequence of the LCDR2 of monoclonal antibody ATG-004 (e.g., SEQ ID NO: 43); and an LCDR3 comprising the amino acid sequence of the LCDR3 of monoclonal antibody ATG-004 (e.g., SEQ ID NO: 48).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-005 (e.g., SEQ ID NO: 19); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-005 (e.g., SEQ ID NO: 28); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-005 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-005 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-005 (e.g., SEQ ID NO: 44); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-005 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-005 (e.g., SEQ ID NO: 19); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-005 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-005 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-005 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-005 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-005 (e.g., SEQ ID NO: 49).

In an embodiment, the antibody molecule comprises: (i) a VH comprising: an HCDR1 comprising the amino acid sequence of the HCDR1 of monoclonal antibody ATG-005 (e.g., SEQ ID NO: 19); an HCDR2 comprising the amino acid sequence of the HCDR2 of monoclonal antibody ATG-005 (e.g., SEQ ID NO: 28); and an HCDR3 comprising the amino acid sequence of the HCDR3 of monoclonal antibody ATG-005 (e.g., SEQ ID NO: 35), and (ii) a VL comprising: an LCDR1 comprising the amino acid sequence of the LCDR1 of monoclonal antibody ATG-005 (e.g., SEQ ID NO: 38); an LCDR2 comprising the amino acid sequence of the LCDR2 of monoclonal antibody ATG-005 (e.g., SEQ ID NO: 44); and an LCDR3 comprising the amino acid sequence of the LCDR3 of monoclonal antibody ATG-005 (e.g., SEQ ID NO: 49).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-006 (e.g., SEQ ID NO: 20); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-006 (e.g., SEQ ID NO: 28); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-006 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-006 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-006 (e.g., SEQ ID NO: 44); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-006 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-006 (e.g., SEQ ID NO: 20); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-006 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-006 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-006 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-006 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-006 (e.g., SEQ ID NO: 49).

In an embodiment, the antibody molecule comprises: (i) a VH comprising: an HCDR1 comprising the amino acid sequence of the HCDR1 of monoclonal antibody ATG-006 (e.g., SEQ ID NO: 20); an HCDR2 comprising the amino acid sequence of the HCDR2 of monoclonal antibody ATG-006 (e.g., SEQ ID NO: 28); and an HCDR3 comprising the amino acid sequence of the HCDR3 of monoclonal antibody ATG-006 (e.g., SEQ ID NO: 35), and (ii) a VL comprising: an LCDR1 comprising the amino acid sequence of the LCDR1 of monoclonal antibody ATG-006 (e.g., SEQ ID NO: 38); an LCDR2 comprising the amino acid sequence of the LCDR2 of monoclonal antibody ATG-006 (e.g., SEQ ID NO: 44); and an LCDR3 comprising the amino acid sequence of the LCDR3 of monoclonal antibody ATG-006 (e.g., SEQ ID NO: 49).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-007 (e.g., SEQ ID NO: 21); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-007 (e.g., SEQ ID NO: 29); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-007 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-007 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-007 (e.g., SEQ ID NO: 44); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-007 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-007 (e.g., SEQ ID NO: 21); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-007 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-007 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-007 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-007 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-007 (e.g., SEQ ID NO: 49).

In an embodiment, the antibody molecule comprises: (i) a VH comprising: an HCDR1 comprising the amino acid sequence of the HCDR1 of monoclonal antibody ATG-007 (e.g., SEQ ID NO: 21); an HCDR2 comprising the amino acid sequence of the HCDR2 of monoclonal antibody ATG-007 (e.g., SEQ ID NO: 29); and an HCDR3 comprising the amino acid sequence of the HCDR3 of monoclonal antibody ATG-007 (e.g., SEQ ID NO: 35), and (ii) a VL comprising: an LCDR1 comprising the amino acid sequence of the LCDR1 of monoclonal antibody ATG-007 (e.g., SEQ ID NO: 38); an LCDR2 comprising the amino acid sequence of the LCDR2 of monoclonal antibody ATG-007 (e.g., SEQ ID NO: 44); and an LCDR3 comprising the amino acid sequence of the LCDR3 of monoclonal antibody ATG-007 (e.g., SEQ ID NO: 49).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-008 (e.g., SEQ ID NO: 22); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-008 (e.g., SEQ ID NO: 29); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-008 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-008 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-008 (e.g., SEQ ID NO: 44); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-008 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-008 (e.g., SEQ ID NO: 22); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-008 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-008 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-008 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-008 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-008 (e.g., SEQ ID NO: 49).

In an embodiment, the antibody molecule comprises: (i) a VH comprising: an HCDR1 comprising the amino acid sequence of the HCDR1 of monoclonal antibody ATG-008 (e.g., SEQ ID NO: 22); an HCDR2 comprising the amino acid sequence of the HCDR2 of monoclonal antibody ATG-008 (e.g., SEQ ID NO: 29); and an HCDR3 comprising the amino acid sequence of the HCDR3 of monoclonal antibody ATG-008 (e.g., SEQ ID NO: 35), and (ii) a VL comprising: an LCDR1 comprising the amino acid sequence of the LCDR1 of monoclonal antibody ATG-008 (e.g., SEQ ID NO: 38); an LCDR2 comprising the amino acid sequence of the LCDR2 of monoclonal antibody ATG-008 (e.g., SEQ ID NO: 44); and an LCDR3 comprising the amino acid sequence of the LCDR3 of monoclonal antibody ATG-008 (e.g., SEQ ID NO: 49).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-012 (e.g., SEQ ID NO: 22); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-012 (e.g., SEQ ID NO: 29); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-012 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-012 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-012 (e.g., SEQ ID NO: 44); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-012 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-012 (e.g., SEQ ID NO: 22); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-012 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-012 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-012 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-012 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-012 (e.g., SEQ ID NO: 49).

In an embodiment, the antibody molecule comprises: (i) a VH comprising: an HCDR1 comprising the amino acid sequence of the HCDR1 of monoclonal antibody ATG-012 (e.g., SEQ ID NO: 22); an HCDR2 comprising the amino acid sequence of the HCDR2 of monoclonal antibody ATG-012 (e.g., SEQ ID NO: 29); and an HCDR3 comprising the amino acid sequence of the HCDR3 of monoclonal antibody ATG-012 (e.g., SEQ ID NO: 35), and (ii) a VL comprising: an LCDR1 comprising the amino acid sequence of the LCDR1 of monoclonal antibody ATG-012 (e.g., SEQ ID NO: 38); an LCDR2 comprising the amino acid sequence of the LCDR2 of monoclonal antibody ATG-012 (e.g., SEQ ID NO: 44); and an LCDR3 comprising the amino acid sequence of the LCDR3 of monoclonal antibody ATG-012 (e.g., SEQ ID NO: 49).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-013 (e.g., SEQ ID NO: 20); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-013 (e.g., SEQ ID NO: 28); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-013 (e.g., SEQ ID NO: 35).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-013 (e.g., SEQ ID NO: 38); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-013 (e.g., SEQ ID NO: 44); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-013 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-013 (e.g., SEQ ID NO: 20); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-013 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-013 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-013 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-013 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-013 (e.g., SEQ ID NO: 49).

In an embodiment, the antibody molecule comprises: (i) a VH comprising: an HCDR1 comprising the amino acid sequence of the HCDR1 of monoclonal antibody ATG-013 (e.g., SEQ ID NO: 20); an HCDR2 comprising the amino acid sequence of the HCDR2 of monoclonal antibody ATG-013 (e.g., SEQ ID NO: 28); and an HCDR3 comprising the amino acid sequence of the HCDR3 of monoclonal antibody ATG-013 (e.g., SEQ ID NO: 35), and (ii) a VL comprising: an LCDR1 comprising the amino acid sequence of the LCDR1 of monoclonal antibody ATG-013 (e.g., SEQ ID NO: 38); an LCDR2 comprising the amino acid sequence of the LCDR2 of monoclonal antibody ATG-013 (e.g., SEQ ID NO: 44); and an LCDR3 comprising the amino acid sequence of the LCDR3 of monoclonal antibody ATG-013 (e.g., SEQ ID NO: 49).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-001 (e.g., SEQ ID NO: 54); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-001 (e.g., SEQ ID NO: 59); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-001 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-001 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-001 (e.g., SEQ ID NO: 76); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-001 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-001 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-001 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-001 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-001 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-001 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-001 (e.g., SEQ ID NO: 81).

In an embodiment, the antibody molecule comprises: (i) a VH comprising: an HCDR1 comprising the amino acid sequence of the HCDR1 of monoclonal antibody ATG-001 (e.g., SEQ ID NO: 54); an HCDR2 comprising the amino acid sequence of the HCDR2 of monoclonal antibody ATG-001 (e.g., SEQ ID NO: 59); and an HCDR3 comprising the amino acid sequence of the HCDR3 of monoclonal antibody ATG-001 (e.g., SEQ ID NO: 66), and (ii) a VL comprising: an LCDR1 comprising the amino acid sequence of the LCDR1 of monoclonal antibody ATG-001 (e.g., SEQ ID NO: 71); an LCDR2 comprising the amino acid sequence of the LCDR2 of monoclonal antibody ATG-001 (e.g., SEQ ID NO: 76); and an LCDR3 comprising the amino acid sequence of the LCDR3 of monoclonal antibody ATG-001 (e.g., SEQ ID NO: 81).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-002 (e.g., SEQ ID NO: 55); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-002 (e.g., SEQ ID NO: 59); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-002 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-002 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-002 (e.g., SEQ ID NO: 76); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-002 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-002 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-002 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-002 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-002 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-002 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-002 (e.g., SEQ ID NO: 81).

In an embodiment, the antibody molecule comprises: (i) a VH comprising: an HCDR1 comprising the amino acid sequence of the HCDR1 of monoclonal antibody ATG-002 (e.g., SEQ ID NO: 55); an HCDR2 comprising the amino acid sequence of the HCDR2 of monoclonal antibody ATG-002 (e.g., SEQ ID NO: 59); and an HCDR3 comprising the amino acid sequence of the HCDR3 of monoclonal antibody ATG-002 (e.g., SEQ ID NO: 66), and (ii) a VL comprising: an LCDR1 comprising the amino acid sequence of the LCDR1 of monoclonal antibody ATG-002 (e.g., SEQ ID NO: 71); an LCDR2 comprising the amino acid sequence of the LCDR2 of monoclonal antibody ATG-002 (e.g., SEQ ID NO: 76); and an LCDR3 comprising the amino acid sequence of the LCDR3 of monoclonal antibody ATG-002 (e.g., SEQ ID NO: 81).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-003 (e.g., SEQ ID NO: 54); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-003 (e.g., SEQ ID NO: 60); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-003 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-003 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-003 (e.g., SEQ ID NO: 76); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-003 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-003 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-003 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-003 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-003 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-003 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-003 (e.g., SEQ ID NO: 81).

In an embodiment, the antibody molecule comprises: (i) a VH comprising: an HCDR1 comprising the amino acid sequence of the HCDR1 of monoclonal antibody ATG-003 (e.g., SEQ ID NO: 54); an HCDR2 comprising the amino acid sequence of the HCDR2 of monoclonal antibody ATG-003 (e.g., SEQ ID NO: 60); and an HCDR3 comprising the amino acid sequence of the HCDR3 of monoclonal antibody ATG-003 (e.g., SEQ ID NO: 66), and (ii) a VL comprising: an LCDR1 comprising the amino acid sequence of the LCDR1 of monoclonal antibody ATG-003 (e.g., SEQ ID NO: 71); an LCDR2 comprising the amino acid sequence of the LCDR2 of monoclonal antibody ATG-003 (e.g., SEQ ID NO: 76); and an LCDR3 comprising the amino acid sequence of the LCDR3 of monoclonal antibody ATG-003 (e.g., SEQ ID NO: 81).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-004 (e.g., SEQ ID NO: 55); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-004 (e.g., SEQ ID NO: 60); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-004 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-004 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-004 (e.g., SEQ ID NO: 76); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-004 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-004 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-004 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-004 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-004 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-004 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-004 (e.g., SEQ ID NO: 81).

In an embodiment, the antibody molecule comprises: (i) a VH comprising: an HCDR1 comprising the amino acid sequence of the HCDR1 of monoclonal antibody ATG-004 (e.g., SEQ ID NO: 55); an HCDR2 comprising the amino acid sequence of the HCDR2 of monoclonal antibody ATG-004 (e.g., SEQ ID NO: 60); and an HCDR3 comprising the amino acid sequence of the HCDR3 of monoclonal antibody ATG-004 (e.g., SEQ ID NO: 66), and (ii) a VL comprising: an LCDR1 comprising the amino acid sequence of the LCDR1 of monoclonal antibody ATG-004 (e.g., SEQ ID NO: 71); an LCDR2 comprising the amino acid sequence of the LCDR2 of monoclonal antibody ATG-004 (e.g., SEQ ID NO: 76); and an LCDR3 comprising the amino acid sequence of the LCDR3 of monoclonal antibody ATG-004 (e.g., SEQ ID NO: 81).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-005 (e.g., SEQ ID NO: 54); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-005 (e.g., SEQ ID NO: 59); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-005 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-005 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-005 (e.g., SEQ ID NO: 77); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-005 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-005 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-005 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-005 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-005 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-005 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-005 (e.g., SEQ ID NO: 82).

In an embodiment, the antibody molecule comprises: (i) a VH comprising: an HCDR1 comprising the amino acid sequence of the HCDR1 of monoclonal antibody ATG-005 (e.g., SEQ ID NO: 54); an HCDR2 comprising the amino acid sequence of the HCDR2 of monoclonal antibody ATG-005 (e.g., SEQ ID NO: 59); and an HCDR3 comprising the amino acid sequence of the HCDR3 of monoclonal antibody ATG-005 (e.g., SEQ ID NO: 66), and (ii) a VL comprising: an LCDR1 comprising the amino acid sequence of the LCDR1 of monoclonal antibody ATG-005 (e.g., SEQ ID NO: 71); an LCDR2 comprising the amino acid sequence of the LCDR2 of monoclonal antibody ATG-005 (e.g., SEQ ID NO: 77); and an LCDR3 comprising the amino acid sequence of the LCDR3 of monoclonal antibody ATG-005 (e.g., SEQ ID NO: 82).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-006 (e.g., SEQ ID NO: 55); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-006 (e.g., SEQ ID NO: 59); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-006 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-006 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-006 (e.g., SEQ ID NO: 77); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-006 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-006 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-006 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-006 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-006 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-006 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-006 (e.g., SEQ ID NO: 82).

In an embodiment, the antibody molecule comprises: (i) a VH comprising: an HCDR1 comprising the amino acid sequence of the HCDR1 of monoclonal antibody ATG-006 (e.g., SEQ ID NO: 55); an HCDR2 comprising the amino acid sequence of the HCDR2 of monoclonal antibody ATG-006 (e.g., SEQ ID NO: 59); and an HCDR3 comprising the amino acid sequence of the HCDR3 of monoclonal antibody ATG-006 (e.g., SEQ ID NO: 66), and (ii) a VL comprising: an LCDR1 comprising the amino acid sequence of the LCDR1 of monoclonal antibody ATG-006 (e.g., SEQ ID NO: 71); an LCDR2 comprising the amino acid sequence of the LCDR2 of monoclonal antibody ATG-006 (e.g., SEQ ID NO: 77); and an LCDR3 comprising the amino acid sequence of the LCDR3 of monoclonal antibody ATG-006 (e.g., SEQ ID NO: 82).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-007 (e.g., SEQ ID NO: 54); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-007 (e.g., SEQ ID NO: 60); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-007 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-007 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-007 (e.g., SEQ ID NO: 77); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-007 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-007 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-007 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-007 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-007 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-007 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-007 (e.g., SEQ ID NO: 82).

In an embodiment, the antibody molecule comprises: (i) a VH comprising: an HCDR1 comprising the amino acid sequence of the HCDR1 of monoclonal antibody ATG-007 (e.g., SEQ ID NO: 54); an HCDR2 comprising the amino acid sequence of the HCDR2 of monoclonal antibody ATG-007 (e.g., SEQ ID NO: 60); and an HCDR3 comprising the amino acid sequence of the HCDR3 of monoclonal antibody ATG-007 (e.g., SEQ ID NO: 66), and (ii) a VL comprising: an LCDR1 comprising the amino acid sequence of the LCDR1 of monoclonal antibody ATG-007 (e.g., SEQ ID NO: 71); an LCDR2 comprising the amino acid sequence of the LCDR2 of monoclonal antibody ATG-007 (e.g., SEQ ID NO: 77); and an LCDR3 comprising the amino acid sequence of the LCDR3 of monoclonal antibody ATG-007 (e.g., SEQ ID NO: 82).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-008 (e.g., SEQ ID NO: 55); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-008 (e.g., SEQ ID NO: 60); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-008 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-008 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-008 (e.g., SEQ ID NO: 77); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-008 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-008 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-008 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-008 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-008 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-008 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-008 (e.g., SEQ ID NO: 82).

In an embodiment, the antibody molecule comprises: (i) a VH comprising: an HCDR1 comprising the amino acid sequence of the HCDR1 of monoclonal antibody ATG-008 (e.g., SEQ ID NO: 55); an HCDR2 comprising the amino acid sequence of the HCDR2 of monoclonal antibody ATG-008 (e.g., SEQ ID NO: 60); and an HCDR3 comprising the amino acid sequence of the HCDR3 of monoclonal antibody ATG-008 (e.g., SEQ ID NO: 66), and (ii) a VL comprising: an LCDR1 comprising the amino acid sequence of the LCDR1 of monoclonal antibody ATG-008 (e.g., SEQ ID NO: 71); an LCDR2 comprising the amino acid sequence of the LCDR2 of monoclonal antibody ATG-008 (e.g., SEQ ID NO: 77); and an LCDR3 comprising the amino acid sequence of the LCDR3 of monoclonal antibody ATG-008 (e.g., SEQ ID NO: 82).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-012 (e.g., SEQ ID NO: 55); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-012 (e.g., SEQ ID NO: 60); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-012 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-012 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-012 (e.g., SEQ ID NO: 77); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-012 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-012 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-012 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-012 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-012 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-012 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-012 (e.g., SEQ ID NO: 82).

In an embodiment, the antibody molecule comprises: (i) a VH comprising: an HCDR1 comprising the amino acid sequence of the HCDR1 of monoclonal antibody ATG-012 (e.g., SEQ ID NO: 55); an HCDR2 comprising the amino acid sequence of the HCDR2 of monoclonal antibody ATG-012 (e.g., SEQ ID NO: 60); and an HCDR3 comprising the amino acid sequence of the HCDR3 of monoclonal antibody ATG-012 (e.g., SEQ ID NO: 66), and (ii) a VL comprising: an LCDR1 comprising the amino acid sequence of the LCDR1 of monoclonal antibody ATG-012 (e.g., SEQ ID NO: 71); an LCDR2 comprising the amino acid sequence of the LCDR2 of monoclonal antibody ATG-012 (e.g., SEQ ID NO: 77); and an LCDR3 comprising the amino acid sequence of the LCDR3 of monoclonal antibody ATG-012 (e.g., SEQ ID NO: 82).

In an embodiment, the antibody molecule comprises a VH, wherein the VH comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-013 (e.g., SEQ ID NO: 55); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-013 (e.g., SEQ ID NO: 59); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-013 (e.g., SEQ ID NO: 66).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-013 (e.g., SEQ ID NO: 71); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-013 (e.g., SEQ ID NO: 77); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-013 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-013 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-013 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-013 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-013 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-013 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-013 (e.g., SEQ ID NO: 82).

In an embodiment, the antibody molecule comprises: (i) a VH comprising: an HCDR1 comprising the amino acid sequence of the HCDR1 of monoclonal antibody ATG-013 (e.g., SEQ ID NO: 55); an HCDR2 comprising the amino acid sequence of the HCDR2 of monoclonal antibody ATG-013 (e.g., SEQ ID NO: 59); and an HCDR3 comprising the amino acid sequence of the HCDR3 of monoclonal antibody ATG-013 (e.g., SEQ ID NO: 66), and (ii) a VL comprising: an LCDR1 comprising the amino acid sequence of the LCDR1 of monoclonal antibody ATG-013 (e.g., SEQ ID NO: 71); an LCDR2 comprising the amino acid sequence of the LCDR2 of monoclonal antibody ATG-013 (e.g., SEQ ID NO: 77); and an LCDR3 comprising the amino acid sequence of the LCDR3 of monoclonal antibody ATG-013 (e.g., SEQ ID NO: 82).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-001 (e.g., SEQ ID NO: 1).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-001 (e.g., SEQ ID NO: 10).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-001 (e.g., SEQ ID NO: 1); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-001 (e.g., SEQ ID NO: 10).

In an embodiment, the antibody molecule comprises: (i) a VH comprising the amino acid sequence of the VH of monoclonal antibody ATG-001 (e.g., SEQ ID NO: 1); and (ii) a VL comprising the amino acid sequence of the VL of monoclonal antibody ATG-001 (e.g., SEQ ID NO: 10).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-002 (e.g., SEQ ID NO: 2).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-002 (e.g., SEQ ID NO: 10).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-002 (e.g., SEQ ID NO: 2); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-002 (e.g., SEQ ID NO: 10).

In an embodiment, the antibody molecule comprises: (i) a VH comprising the amino acid sequence of the VH of monoclonal antibody ATG-002 (e.g., SEQ ID NO: 2); and (ii) a VL comprising the amino acid sequence of the VL of monoclonal antibody ATG-002 (e.g., SEQ ID NO: 10).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-003 (e.g., SEQ ID NO: 3).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-003 (e.g., SEQ ID NO: 10).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-003 (e.g., SEQ ID NO: 3); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-003 (e.g., SEQ ID NO: 10).

In an embodiment, the antibody molecule comprises: (i) a VH comprising the amino acid sequence of the VH of monoclonal antibody ATG-003 (e.g., SEQ ID NO: 3); and (ii) a VL comprising the amino acid sequence of the VL of monoclonal antibody ATG-003 (e.g., SEQ ID NO: 10).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-004 (e.g., SEQ ID NO: 4).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-004 (e.g., SEQ ID NO: 10).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-004 (e.g., SEQ ID NO: 4); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-004 (e.g., SEQ ID NO: 10).

In an embodiment, the antibody molecule comprises: (i) a VH comprising the amino acid sequence of the VH of monoclonal antibody ATG-004 (e.g., SEQ ID NO: 4); and (ii) a VL comprising the amino acid sequence of the VL of monoclonal antibody ATG-004 (e.g., SEQ ID NO: 10).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-005 (e.g., SEQ ID NO: 1).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-005 (e.g., SEQ ID NO: 11).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-005 (e.g., SEQ ID NO: 1); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-005 (e.g., SEQ ID NO: 11).

In an embodiment, the antibody molecule comprises: (i) a VH comprising the amino acid sequence of the VH of monoclonal antibody ATG-005 (e.g., SEQ ID NO: 1); and (ii) a VL comprising the amino acid sequence of the VL of monoclonal antibody ATG-005 (e.g., SEQ ID NO: 11).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-006 (e.g., SEQ ID NO: 2).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-006 (e.g., SEQ ID NO: 11).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-006 (e.g., SEQ ID NO: 2); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-006 (e.g., SEQ ID NO: 11).

In an embodiment, the antibody molecule comprises: (i) a VH comprising the amino acid sequence of the VH of monoclonal antibody ATG-006 (e.g., SEQ ID NO: 2); and (ii) a VL comprising the amino acid sequence of the VL of monoclonal antibody ATG-006 (e.g., SEQ ID NO: 11).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-007 (e.g., SEQ ID NO: 3).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-007 (e.g., SEQ ID NO: 11).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-007 (e.g., SEQ ID NO: 3); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-007 (e.g., SEQ ID NO: 11).

In an embodiment, the antibody molecule comprises: (i) a VH comprising the amino acid sequence of the VH of monoclonal antibody ATG-007 (e.g., SEQ ID NO: 3); and (ii) a VL comprising the amino acid sequence of the VL of monoclonal antibody ATG-007 (e.g., SEQ ID NO: 11).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-008 (e.g., SEQ ID NO: 4).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-008 (e.g., SEQ ID NO: 11).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-008 (e.g., SEQ ID NO: 4); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-008 (e.g., SEQ ID NO: 11).

In an embodiment, the antibody molecule comprises: (i) a VH comprising the amino acid sequence of the VH of monoclonal antibody ATG-008 (e.g., SEQ ID NO: 4); and (ii) a VL comprising the amino acid sequence of the VL of monoclonal antibody ATG-008 (e.g., SEQ ID NO: 11).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-012 (e.g., SEQ ID NO: 4).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-012 (e.g., SEQ ID NO: 11).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-012 (e.g., SEQ ID NO: 4); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-012 (e.g., SEQ ID NO: 11).

In an embodiment, the antibody molecule comprises: (i) a VH comprising the amino acid sequence of the VH of monoclonal antibody ATG-012 (e.g., SEQ ID NO: 4); and (ii) a VL comprising the amino acid sequence of the VL of monoclonal antibody ATG-012 (e.g., SEQ ID NO: 11).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-013 (e.g., SEQ ID NO: 2).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-013 (e.g., SEQ ID NO: 11).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-013 (e.g., SEQ ID NO: 2); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-013 (e.g., SEQ ID NO: 11).

In an embodiment, the antibody molecule comprises: (i) a VH comprising the amino acid sequence of the VH of monoclonal antibody ATG-013 (e.g., SEQ ID NO: 2); and (ii) a VL comprising the amino acid sequence of the VL of monoclonal antibody ATG-013 (e.g., SEQ ID NO: 11).

In an embodiment the antibody molecule is monoclonal antibody ATG-001, ATG-002, ATG-003, ATG-004, ATG-005, ATG-006, ATG-007, ATG-008, ATG-012, or ATG-013.

In an embodiment, the antibody molecule comprises one or more framework regions derived from a human framework germline sequence.

In an embodiment, the antibody molecule comprises a VH described in Table 1. In an embodiment, the antibody molecule comprises a VL described in Table 1. In an embodiment, the antibody molecule comprises a VH and a VL described in Table 1. In an embodiment, the antibody molecule comprises one, two, or three CDRs of a VH described in Table 1. In an embodiment, the antibody molecule comprises one, two, or three CDRs of a VL described in Table 1. In an embodiment, the antibody molecule comprises one, two, or three CDRs of a VH described in Table 1, and one, two, or three CDRs of a VL described in Table 1.

In an embodiment, the antibody molecule comprises two VHs and two VLs. In an embodiment, the antibody molecule comprises an antigen-binding fragment. In an embodiment, the antibody molecule comprises a Fab, F(ab′)2, Fv, scFv, or Fd.

In an embodiment, the antibody molecule is an IgG antibody molecule, e.g., comprising a heavy chain constant region of IgG, e.g., chosen from IgG1, IgG2, IgG3, or IgG4, e.g., IgG2 or IgG4. In an embodiment, the antibody molecule is an IgG1 antibody molecule, e.g., having an IgG1 constant region described herein. In another embodiment, the antibody molecule is an IgG2 antibody molecule e.g., having an IgG2 constant region described herein. In an embodiment, the antibody molecule is an IgG3 antibody molecule, e.g., having an IgG3 constant region described herein. In another embodiment, the antibody molecule is an IgG4 antibody molecule e.g., having an IgG4 constant region described herein. In another embodiment, the antibody molecule has a chimeric constant region comprising of IgG2, IgG3 and/or IgG4 isotypes. In an embodiment, the heavy chain constant region comprises one or more amino acid modifications in the hinge, CH2 or CH3 region. In an embodiment, the antibody molecule comprises a light chain constant region of kappa or lambda light chain.

In an embodiment, the antibody molecule comprises an Fc region. In an embodiment, the Fc region comprises one or both of Met-428-Leu and Asn-434-Ser substitutions at residues corresponding to methionine 428 and asparagine 434, respectively, each in EU numbering. In an embodiment, the Fc region comprises one, two or three of Met-252-Tyr, Ser-254-Thr and Thr-256-Glu substitutions at residues corresponding to methionine 252, serine 254 and threonine 256, respectively, each in EU numbering.

In an aspect, the disclosure features an anti-C5 antibody molecule described herein, e.g., a synthetic or isolated anti-C5 antibody molecule described herein.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-001 (e.g., SEQ ID NO: 19 or 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-001 (e.g., SEQ ID NO: 28 or 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-001 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-001 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-001 (e.g., SEQ ID NO: 43 or 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-001 (e.g., SEQ ID NO: 48 or 81).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-002 (e.g., SEQ ID NO: 20 or 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-002 (e.g., SEQ ID NO: 28 or 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-002 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-002 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-002 (e.g., SEQ ID NO: 43 or 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-002 (e.g., SEQ ID NO: 48 or 81).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-003 (e.g., SEQ ID NO: 21 or 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-003 (e.g., SEQ ID NO: 29 or 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-003 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-003 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-003 (e.g., SEQ ID NO: 43 or 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-003 (e.g., SEQ ID NO: 48 or 81).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-004 (e.g., SEQ ID NO: 22 or 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-004 (e.g., SEQ ID NO: 29 or 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-004 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-004 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-004 (e.g., SEQ ID NO: 43 or 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-004 (e.g., SEQ ID NO: 48 or 81).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-005 (e.g., SEQ ID NO: 19 or 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-005 (e.g., SEQ ID NO: 28 or 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-005 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-005 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-005 (e.g., SEQ ID NO: 44 or 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-005 (e.g., SEQ ID NO: 49 or 82).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-006 (e.g., SEQ ID NO: 20 or 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-006 (e.g., SEQ ID NO: 28 or 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-006 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-006 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-006 (e.g., SEQ ID NO: 44 or 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-006 (e.g., SEQ ID NO: 49 or 82).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-007 (e.g., SEQ ID NO: 21 or 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-007 (e.g., SEQ ID NO: 29 or 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-007 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-007 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-007 (e.g., SEQ ID NO: 44 or 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-007 (e.g., SEQ ID NO: 49 or 82).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-008 (e.g., SEQ ID NO: 22 or 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-008 (e.g., SEQ ID NO: 29 or 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-008 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-008 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-008 (e.g., SEQ ID NO: 44 or 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-008 (e.g., SEQ ID NO: 49 or 82).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-012 (e.g., SEQ ID NO: 22 or 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-012 (e.g., SEQ ID NO: 29 or 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-012 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-012 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-012 (e.g., SEQ ID NO: 44 or 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-012 (e.g., SEQ ID NO: 49 or 82).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-013 (e.g., SEQ ID NO: 20 or 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-013 (e.g., SEQ ID NO: 28 or 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-013 (e.g., SEQ ID NO: 35 or 66), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-013 (e.g., SEQ ID NO: 38 or 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-013 (e.g., SEQ ID NO: 44 or 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-013 (e.g., SEQ ID NO: 49 or 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-001 (e.g., SEQ ID NO: 19); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-001 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-001 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-001 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-001 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-001 (e.g., SEQ ID NO: 48).

In an embodiment, the antibody molecule comprises: (i) a VH comprising: an HCDR1 comprising the amino acid sequence of the HCDR1 of monoclonal antibody ATG-001 (e.g., SEQ ID NO: 19); an HCDR2 comprising the amino acid sequence of the HCDR2 of monoclonal antibody ATG-001 (e.g., SEQ ID NO: 28); and an HCDR3 comprising the amino acid sequence of the HCDR3 of monoclonal antibody ATG-001 (e.g., SEQ ID NO: 35), and (ii) a VL comprising: an LCDR1 comprising the amino acid sequence of the LCDR1 of monoclonal antibody ATG-001 (e.g., SEQ ID NO: 38); an LCDR2 comprising the amino acid sequence of the LCDR2 of monoclonal antibody ATG-001 (e.g., SEQ ID NO: 43); and an LCDR3 comprising the amino acid sequence of the LCDR3 of monoclonal antibody ATG-001 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-002 (e.g., SEQ ID NO: 20); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-002 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-002 (e.g., SEQ ID NO: 35), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-002 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-002 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-002 (e.g., SEQ ID NO: 48).

In an embodiment, the antibody molecule comprises: (i) a VH comprising: an HCDR1 comprising the amino acid sequence of the HCDR1 of monoclonal antibody ATG-002 (e.g., SEQ ID NO: 20); an HCDR2 comprising the amino acid sequence of the HCDR2 of monoclonal antibody ATG-002 (e.g., SEQ ID NO: 28); and an HCDR3 comprising the amino acid sequence of the HCDR3 of monoclonal antibody ATG-002 (e.g., SEQ ID NO: 35), and (ii) a VL comprising: an LCDR1 comprising the amino acid sequence of the LCDR1 of monoclonal antibody ATG-002 (e.g., SEQ ID NO: 38); an LCDR2 comprising the amino acid sequence of the LCDR2 of monoclonal antibody ATG-002 (e.g., SEQ ID NO: 43); and an LCDR3 comprising the amino acid sequence of the LCDR3 of monoclonal antibody ATG-002 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-003 (e.g., SEQ ID NO: 21); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-003 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-003 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-003 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-003 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-003 (e.g., SEQ ID NO: 48).

In an embodiment, the antibody molecule comprises: (i) a VH comprising: an HCDR1 comprising the amino acid sequence of the HCDR1 of monoclonal antibody ATG-003 (e.g., SEQ ID NO: 21); an HCDR2 comprising the amino acid sequence of the HCDR2 of monoclonal antibody ATG-003 (e.g., SEQ ID NO: 29); and an HCDR3 comprising the amino acid sequence of the HCDR3 of monoclonal antibody ATG-003 (e.g., SEQ ID NO: 35), and (ii) a VL comprising: an LCDR1 comprising the amino acid sequence of the LCDR1 of monoclonal antibody ATG-003 (e.g., SEQ ID NO: 38); an LCDR2 comprising the amino acid sequence of the LCDR2 of monoclonal antibody ATG-003 (e.g., SEQ ID NO: 43); and an LCDR3 comprising the amino acid sequence of the LCDR3 of monoclonal antibody ATG-003 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-004 (e.g., SEQ ID NO: 22); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-004 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-004 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-004 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-004 (e.g., SEQ ID NO: 43); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-004 (e.g., SEQ ID NO: 48).

In an embodiment, the antibody molecule comprises: (i) a VH comprising: an HCDR1 comprising the amino acid sequence of the HCDR1 of monoclonal antibody ATG-004 (e.g., SEQ ID NO: 22); an HCDR2 comprising the amino acid sequence of the HCDR2 of monoclonal antibody ATG-004 (e.g., SEQ ID NO: 29); and an HCDR3 comprising the amino acid sequence of the HCDR3 of monoclonal antibody ATG-004 (e.g., SEQ ID NO: 35), and (ii) a VL comprising: an LCDR1 comprising the amino acid sequence of the LCDR1 of monoclonal antibody ATG-004 (e.g., SEQ ID NO: 38); an LCDR2 comprising the amino acid sequence of the LCDR2 of monoclonal antibody ATG-004 (e.g., SEQ ID NO: 43); and an LCDR3 comprising the amino acid sequence of the LCDR3 of monoclonal antibody ATG-004 (e.g., SEQ ID NO: 48).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-005 (e.g., SEQ ID NO: 19); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-005 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-005 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-005 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-005 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-005 (e.g., SEQ ID NO: 49).

In an embodiment, the antibody molecule comprises: (i) a VH comprising: an HCDR1 comprising the amino acid sequence of the HCDR1 of monoclonal antibody ATG-005 (e.g., SEQ ID NO: 19); an HCDR2 comprising the amino acid sequence of the HCDR2 of monoclonal antibody ATG-005 (e.g., SEQ ID NO: 28); and an HCDR3 comprising the amino acid sequence of the HCDR3 of monoclonal antibody ATG-005 (e.g., SEQ ID NO: 35), and (ii) a VL comprising: an LCDR1 comprising the amino acid sequence of the LCDR1 of monoclonal antibody ATG-005 (e.g., SEQ ID NO: 38); an LCDR2 comprising the amino acid sequence of the LCDR2 of monoclonal antibody ATG-005 (e.g., SEQ ID NO: 44); and an LCDR3 comprising the amino acid sequence of the LCDR3 of monoclonal antibody ATG-005 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-006 (e.g., SEQ ID NO: 20); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-006 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-006 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-006 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-006 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-006 (e.g., SEQ ID NO: 49).

In an embodiment, the antibody molecule comprises: (i) a VH comprising: an HCDR1 comprising the amino acid sequence of the HCDR1 of monoclonal antibody ATG-006 (e.g., SEQ ID NO: 20); an HCDR2 comprising the amino acid sequence of the HCDR2 of monoclonal antibody ATG-006 (e.g., SEQ ID NO: 28); and an HCDR3 comprising the amino acid sequence of the HCDR3 of monoclonal antibody ATG-006 (e.g., SEQ ID NO: 35), and (ii) a VL comprising: an LCDR1 comprising the amino acid sequence of the LCDR1 of monoclonal antibody ATG-006 (e.g., SEQ ID NO: 38); an LCDR2 comprising the amino acid sequence of the LCDR2 of monoclonal antibody ATG-006 (e.g., SEQ ID NO: 44); and an LCDR3 comprising the amino acid sequence of the LCDR3 of monoclonal antibody ATG-006 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-007 (e.g., SEQ ID NO: 21); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-007 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-007 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-007 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-007 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-007 (e.g., SEQ ID NO: 49).

In an embodiment, the antibody molecule comprises: (i) a VH comprising: an HCDR1 comprising the amino acid sequence of the HCDR1 of monoclonal antibody ATG-007 (e.g., SEQ ID NO: 21); an HCDR2 comprising the amino acid sequence of the HCDR2 of monoclonal antibody ATG-007 (e.g., SEQ ID NO: 29); and an HCDR3 comprising the amino acid sequence of the HCDR3 of monoclonal antibody ATG-007 (e.g., SEQ ID NO: 35), and (ii) a VL comprising: an LCDR1 comprising the amino acid sequence of the LCDR1 of monoclonal antibody ATG-007 (e.g., SEQ ID NO: 38); an LCDR2 comprising the amino acid sequence of the LCDR2 of monoclonal antibody ATG-007 (e.g., SEQ ID NO: 44); and an LCDR3 comprising the amino acid sequence of the LCDR3 of monoclonal antibody ATG-007 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-008 (e.g., SEQ ID NO: 22); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-008 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-008 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-008 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-008 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-008 (e.g., SEQ ID NO: 49).

In an embodiment, the antibody molecule comprises: (i) a VH comprising: an HCDR1 comprising the amino acid sequence of the HCDR1 of monoclonal antibody ATG-008 (e.g., SEQ ID NO: 22); an HCDR2 comprising the amino acid sequence of the HCDR2 of monoclonal antibody ATG-008 (e.g., SEQ ID NO: 29); and an HCDR3 comprising the amino acid sequence of the HCDR3 of monoclonal antibody ATG-008 (e.g., SEQ ID NO: 35), and (ii) a VL comprising: an LCDR1 comprising the amino acid sequence of the LCDR1 of monoclonal antibody ATG-008 (e.g., SEQ ID NO: 38); an LCDR2 comprising the amino acid sequence of the LCDR2 of monoclonal antibody ATG-008 (e.g., SEQ ID NO: 44); and an LCDR3 comprising the amino acid sequence of the LCDR3 of monoclonal antibody ATG-008 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-012 (e.g., SEQ ID NO: 22); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-012 (e.g., SEQ ID NO: 29); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-012 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-012 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-012 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-012 (e.g., SEQ ID NO: 49).

In an embodiment, the antibody molecule comprises: (i) a VH comprising: an HCDR1 comprising the amino acid sequence of the HCDR1 of monoclonal antibody ATG-012 (e.g., SEQ ID NO: 22); an HCDR2 comprising the amino acid sequence of the HCDR2 of monoclonal antibody ATG-012 (e.g., SEQ ID NO: 29); and an HCDR3 comprising the amino acid sequence of the HCDR3 of monoclonal antibody ATG-012 (e.g., SEQ ID NO: 35), and (ii) a VL comprising: an LCDR1 comprising the amino acid sequence of the LCDR1 of monoclonal antibody ATG-012 (e.g., SEQ ID NO: 38); an LCDR2 comprising the amino acid sequence of the LCDR2 of monoclonal antibody ATG-012 (e.g., SEQ ID NO: 44); and an LCDR3 comprising the amino acid sequence of the LCDR3 of monoclonal antibody ATG-012 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-013 (e.g., SEQ ID NO: 20); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-013 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-013 (e.g., SEQ ID NO: 35), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-013 (e.g., SEQ ID NO: 38); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-013 (e.g., SEQ ID NO: 44); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-013 (e.g., SEQ ID NO: 49).

In an embodiment, the antibody molecule comprises: (i) a VH comprising: an HCDR1 comprising the amino acid sequence of the HCDR1 of monoclonal antibody ATG-013 (e.g., SEQ ID NO: 20); an HCDR2 comprising the amino acid sequence of the HCDR2 of monoclonal antibody ATG-013 (e.g., SEQ ID NO: 28); and an HCDR3 comprising the amino acid sequence of the HCDR3 of monoclonal antibody ATG-013 (e.g., SEQ ID NO: 35), and (ii) a VL comprising: an LCDR1 comprising the amino acid sequence of the LCDR1 of monoclonal antibody ATG-013 (e.g., SEQ ID NO: 38); an LCDR2 comprising the amino acid sequence of the LCDR2 of monoclonal antibody ATG-013 (e.g., SEQ ID NO: 44); and an LCDR3 comprising the amino acid sequence of the LCDR3 of monoclonal antibody ATG-013 (e.g., SEQ ID NO: 49).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-001 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-001 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-001 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-001 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-001 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-001 (e.g., SEQ ID NO: 81).

In an embodiment, the antibody molecule comprises: (i) a VH comprising: an HCDR1 comprising the amino acid sequence of the HCDR1 of monoclonal antibody ATG-001 (e.g., SEQ ID NO: 54); an HCDR2 comprising the amino acid sequence of the HCDR2 of monoclonal antibody ATG-001 (e.g., SEQ ID NO: 59); and an HCDR3 comprising the amino acid sequence of the HCDR3 of monoclonal antibody ATG-001 (e.g., SEQ ID NO: 66), and (ii) a VL comprising: an LCDR1 comprising the amino acid sequence of the LCDR1 of monoclonal antibody ATG-001 (e.g., SEQ ID NO: 71); an LCDR2 comprising the amino acid sequence of the LCDR2 of monoclonal antibody ATG-001 (e.g., SEQ ID NO: 76); and an LCDR3 comprising the amino acid sequence of the LCDR3 of monoclonal antibody ATG-001 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-002 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-002 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-002 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-002 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-002 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-002 (e.g., SEQ ID NO: 81).

In an embodiment, the antibody molecule comprises: (i) a VH comprising: an HCDR1 comprising the amino acid sequence of the HCDR1 of monoclonal antibody ATG-002 (e.g., SEQ ID NO: 55); an HCDR2 comprising the amino acid sequence of the HCDR2 of monoclonal antibody ATG-002 (e.g., SEQ ID NO: 59); and an HCDR3 comprising the amino acid sequence of the HCDR3 of monoclonal antibody ATG-002 (e.g., SEQ ID NO: 66), and (ii) a VL comprising: an LCDR1 comprising the amino acid sequence of the LCDR1 of monoclonal antibody ATG-002 (e.g., SEQ ID NO: 71); an LCDR2 comprising the amino acid sequence of the LCDR2 of monoclonal antibody ATG-002 (e.g., SEQ ID NO: 76); and an LCDR3 comprising the amino acid sequence of the LCDR3 of monoclonal antibody ATG-002 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-003 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-003 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-003 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-003 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-003 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-003 (e.g., SEQ ID NO: 81).

In an embodiment, the antibody molecule comprises: (i) a VH comprising: an HCDR1 comprising the amino acid sequence of the HCDR1 of monoclonal antibody ATG-003 (e.g., SEQ ID NO: 54); an HCDR2 comprising the amino acid sequence of the HCDR2 of monoclonal antibody ATG-003 (e.g., SEQ ID NO: 60); and an HCDR3 comprising the amino acid sequence of the HCDR3 of monoclonal antibody ATG-003 (e.g., SEQ ID NO: 66), and (ii) a VL comprising: an LCDR1 comprising the amino acid sequence of the LCDR1 of monoclonal antibody ATG-003 (e.g., SEQ ID NO: 71); an LCDR2 comprising the amino acid sequence of the LCDR2 of monoclonal antibody ATG-003 (e.g., SEQ ID NO: 76); and an LCDR3 comprising the amino acid sequence of the LCDR3 of monoclonal antibody ATG-003 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-004 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-004 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-004 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-004 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-004 (e.g., SEQ ID NO: 76); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-004 (e.g., SEQ ID NO: 81).

In an embodiment, the antibody molecule comprises: (i) a VH comprising: an HCDR1 comprising the amino acid sequence of the HCDR1 of monoclonal antibody ATG-004 (e.g., SEQ ID NO: 55); an HCDR2 comprising the amino acid sequence of the HCDR2 of monoclonal antibody ATG-004 (e.g., SEQ ID NO: 60); and an HCDR3 comprising the amino acid sequence of the HCDR3 of monoclonal antibody ATG-004 (e.g., SEQ ID NO: 66), and (ii) a VL comprising: an LCDR1 comprising the amino acid sequence of the LCDR1 of monoclonal antibody ATG-004 (e.g., SEQ ID NO: 71); an LCDR2 comprising the amino acid sequence of the LCDR2 of monoclonal antibody ATG-004 (e.g., SEQ ID NO: 76); and an LCDR3 comprising the amino acid sequence of the LCDR3 of monoclonal antibody ATG-004 (e.g., SEQ ID NO: 81).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-005 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-005 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-005 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-005 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-005 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-005 (e.g., SEQ ID NO: 82).

In an embodiment, the antibody molecule comprises: (i) a VH comprising: an HCDR1 comprising the amino acid sequence of the HCDR1 of monoclonal antibody ATG-005 (e.g., SEQ ID NO: 54); an HCDR2 comprising the amino acid sequence of the HCDR2 of monoclonal antibody ATG-005 (e.g., SEQ ID NO: 59); and an HCDR3 comprising the amino acid sequence of the HCDR3 of monoclonal antibody ATG-005 (e.g., SEQ ID NO: 66), and (ii) a VL comprising: an LCDR1 comprising the amino acid sequence of the LCDR1 of monoclonal antibody ATG-005 (e.g., SEQ ID NO: 71); an LCDR2 comprising the amino acid sequence of the LCDR2 of monoclonal antibody ATG-005 (e.g., SEQ ID NO: 77); and an LCDR3 comprising the amino acid sequence of the LCDR3 of monoclonal antibody ATG-005 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-006 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-006 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-006 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-006 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-006 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-006 (e.g., SEQ ID NO: 82).

In an embodiment, the antibody molecule comprises: (i) a VH comprising: an HCDR1 comprising the amino acid sequence of the HCDR1 of monoclonal antibody ATG-006 (e.g., SEQ ID NO: 55); an HCDR2 comprising the amino acid sequence of the HCDR2 of monoclonal antibody ATG-006 (e.g., SEQ ID NO: 59); and an HCDR3 comprising the amino acid sequence of the HCDR3 of monoclonal antibody ATG-006 (e.g., SEQ ID NO: 66), and (ii) a VL comprising: an LCDR1 comprising the amino acid sequence of the LCDR1 of monoclonal antibody ATG-006 (e.g., SEQ ID NO: 71); an LCDR2 comprising the amino acid sequence of the LCDR2 of monoclonal antibody ATG-006 (e.g., SEQ ID NO: 77); and an LCDR3 comprising the amino acid sequence of the LCDR3 of monoclonal antibody ATG-006 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-007 (e.g., SEQ ID NO: 54); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-007 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-007 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-007 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-007 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-007 (e.g., SEQ ID NO: 82).

In an embodiment, the antibody molecule comprises: (i) a VH comprising: an HCDR1 comprising the amino acid sequence of the HCDR1 of monoclonal antibody ATG-007 (e.g., SEQ ID NO: 54); an HCDR2 comprising the amino acid sequence of the HCDR2 of monoclonal antibody ATG-007 (e.g., SEQ ID NO: 60); and an HCDR3 comprising the amino acid sequence of the HCDR3 of monoclonal antibody ATG-007 (e.g., SEQ ID NO: 66), and (ii) a VL comprising: an LCDR1 comprising the amino acid sequence of the LCDR1 of monoclonal antibody ATG-007 (e.g., SEQ ID NO: 71); an LCDR2 comprising the amino acid sequence of the LCDR2 of monoclonal antibody ATG-007 (e.g., SEQ ID NO: 77); and an LCDR3 comprising the amino acid sequence of the LCDR3 of monoclonal antibody ATG-007 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-008 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-008 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-008 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-008 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-008 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-008 (e.g., SEQ ID NO: 82).

In an embodiment, the antibody molecule comprises: (i) a VH comprising: an HCDR1 comprising the amino acid sequence of the HCDR1 of monoclonal antibody ATG-008 (e.g., SEQ ID NO: 55); an HCDR2 comprising the amino acid sequence of the HCDR2 of monoclonal antibody ATG-008 (e.g., SEQ ID NO: 60); and an HCDR3 comprising the amino acid sequence of the HCDR3 of monoclonal antibody ATG-008 (e.g., SEQ ID NO: 66), and (ii) a VL comprising: an LCDR1 comprising the amino acid sequence of the LCDR1 of monoclonal antibody ATG-008 (e.g., SEQ ID NO: 71); an LCDR2 comprising the amino acid sequence of the LCDR2 of monoclonal antibody ATG-008 (e.g., SEQ ID NO: 77); and an LCDR3 comprising the amino acid sequence of the LCDR3 of monoclonal antibody ATG-008 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-012 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-012 (e.g., SEQ ID NO: 60); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-012 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-012 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-012 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-012 (e.g., SEQ ID NO: 82).

In an embodiment, the antibody molecule comprises: (i) a VH comprising: an HCDR1 comprising the amino acid sequence of the HCDR1 of monoclonal antibody ATG-012 (e.g., SEQ ID NO: 55); an HCDR2 comprising the amino acid sequence of the HCDR2 of monoclonal antibody ATG-012 (e.g., SEQ ID NO: 60); and an HCDR3 comprising the amino acid sequence of the HCDR3 of monoclonal antibody ATG-012 (e.g., SEQ ID NO: 66), and (ii) a VL comprising: an LCDR1 comprising the amino acid sequence of the LCDR1 of monoclonal antibody ATG-012 (e.g., SEQ ID NO: 71); an LCDR2 comprising the amino acid sequence of the LCDR2 of monoclonal antibody ATG-012 (e.g., SEQ ID NO: 77); and an LCDR3 comprising the amino acid sequence of the LCDR3 of monoclonal antibody ATG-012 (e.g., SEQ ID NO: 82).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ATG-013 (e.g., SEQ ID NO: 55); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ATG-013 (e.g., SEQ ID NO: 59); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ATG-013 (e.g., SEQ ID NO: 66), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ATG-013 (e.g., SEQ ID NO: 71); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ATG-013 (e.g., SEQ ID NO: 77); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ATG-013 (e.g., SEQ ID NO: 82).

In an embodiment, the antibody molecule comprises: (i) a VH comprising: an HCDR1 comprising the amino acid sequence of the HCDR1 of monoclonal antibody ATG-013 (e.g., SEQ ID NO: 55); an HCDR2 comprising the amino acid sequence of the HCDR2 of monoclonal antibody ATG-013 (e.g., SEQ ID NO: 59); and an HCDR3 comprising the amino acid sequence of the HCDR3 of monoclonal antibody ATG-013 (e.g., SEQ ID NO: 66), and (ii) a VL comprising: an LCDR1 comprising the amino acid sequence of the LCDR1 of monoclonal antibody ATG-013 (e.g., SEQ ID NO: 71); an LCDR2 comprising the amino acid sequence of the LCDR2 of monoclonal antibody ATG-013 (e.g., SEQ ID NO: 77); and an LCDR3 comprising the amino acid sequence of the LCDR3 of monoclonal antibody ATG-013 (e.g., SEQ ID NO: 82).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-001 (e.g., SEQ ID NO: 1); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-001 (e.g., SEQ ID NO: 10).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-002 (e.g., SEQ ID NO: 2); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-002 (e.g., SEQ ID NO: 10).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-003 (e.g., SEQ ID NO: 3); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-003 (e.g., SEQ ID NO: 10).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-004 (e.g., SEQ ID NO: 4); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-004 (e.g., SEQ ID NO: 10).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-005 (e.g., SEQ ID NO: 1); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-005 (e.g., SEQ ID NO: 11).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-006 (e.g., SEQ ID NO: 2); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-006 (e.g., SEQ ID NO: 11).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-007 (e.g., SEQ ID NO: 3); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-007 (e.g., SEQ ID NO: 11).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-008 (e.g., SEQ ID NO: 4); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-008 (e.g., SEQ ID NO: 11).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-012 (e.g., SEQ ID NO: 4); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-012 (e.g., SEQ ID NO: 11).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ATG-013 (e.g., SEQ ID NO: 2); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ATG-013 (e.g., SEQ ID NO: 11).

In an embodiment, the antibody molecule comprises a VH encoded by the nucleotide sequence of SEQ ID NO: 100 (or a nucleotide sequence substantially identical thereto) or a VL encoded by the nucleotide sequence of SEQ ID NO: 101 (or a nucleotide sequence substantially identical thereto), or both. In an embodiment, the antibody molecule comprises a VH encoded by the nucleotide sequence of SEQ ID NO: 102 (or a nucleotide sequence substantially identical thereto) or a VL encoded by the nucleotide sequence of SEQ ID NO: 103 (or a nucleotide sequence substantially identical thereto), or both. In an embodiment, the antibody molecule comprises a VH encoded by the nucleotide sequence of SEQ ID NO: 104 (or a nucleotide sequence substantially identical thereto) or a VL encoded by the nucleotide sequence of SEQ ID NO: 105 (or a nucleotide sequence substantially identical thereto), or both. In an embodiment, the antibody molecule comprises a VH encoded by the nucleotide sequence of SEQ ID NO: 106 (or a nucleotide sequence substantially identical thereto) or a VL encoded by the nucleotide sequence of SEQ ID NO: 107 (or a nucleotide sequence substantially identical thereto), or both. In an embodiment, the antibody molecule comprises a VH encoded by the nucleotide sequence of SEQ ID NO: 108 (or a nucleotide sequence substantially identical thereto) or a VL encoded by the nucleotide sequence of SEQ ID NO: 109 (or a nucleotide sequence substantially identical thereto), or both. In an embodiment, the antibody molecule comprises a VH encoded by the nucleotide sequence of SEQ ID NO: 110 (or a nucleotide sequence substantially identical thereto) or a VL encoded by the nucleotide sequence of SEQ ID NO: 111 (or a nucleotide sequence substantially identical thereto), or both. In an embodiment, the antibody molecule comprises a VH encoded by the nucleotide sequence of SEQ ID NO: 112 (or a nucleotide sequence substantially identical thereto) or a VL encoded by the nucleotide sequence of SEQ ID NO: 113 (or a nucleotide sequence substantially identical thereto), or both. In an embodiment, the antibody molecule comprises a VH encoded by the nucleotide sequence of SEQ ID NO: 114 (or a nucleotide sequence substantially identical thereto) or a VL encoded by the nucleotide sequence of SEQ ID NO: 115 (or a nucleotide sequence substantially identical thereto), or both.

In an embodiment, the antibody molecule is monoclonal antibody ATG-001, ATG-002, ATG-003, ATG-004, ATG-005, ATG-006, ATG-007, ATG-008, ATG-012, or ATG-013. In an embodiment, the antibody molecule comprises a VH comprising the amino acid sequence of any of SEQ ID NO: 1-9, a VL comprising the amino acid sequence of any of SEQ ID NO: 10-15, or both.

In an embodiment, the antibody molecule is any of antibodies ATG-001, ATG-002, ATG-003, ATG-004, ATG-005, ATG-006, ATG-007, ATG-008, ATG-012, or ATG-013.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In an embodiment, the antibody molecule comprises two heavy chain variable regions and two light chain variable regions. In an embodiment, the antibody molecule comprises an antigen-binding fragment. In an embodiment, the antibody molecule comprises a Fab, F(ab′)2, Fv, scFv, or Fd.

In an embodiment, the antibody molecule is an IgG antibody molecule, e.g., comprising a heavy chain constant region of IgG, e.g., chosen from IgG1, IgG2, IgG3, or IgG4, e.g., IgG2 or IgG4. In an embodiment, the antibody molecule is an IgG1 antibody molecule, e.g., having an IgG1 constant region described herein. In another embodiment, the antibody molecule is an IgG2 antibody molecule e.g., having an IgG2 constant region described herein. In an embodiment, the antibody molecule is an IgG3 antibody molecule, e.g., having an IgG3 constant region described herein. In another embodiment, the antibody molecule is an IgG4 antibody molecule e.g., having an IgG4 constant region described herein. In another embodiment, the antibody molecule has a chimeric constant region comprising of IgG2, IgG3 and/or IgG4 isotypes. In an embodiment, the heavy chain constant region comprises one or more amino acid modifications in the hinge, CH2 or CH3 region. In an embodiment, the antibody molecule comprises a light chain constant region of kappa or lambda light chain.

In an embodiment, the antibody molecule comprises an Fc region. In an embodiment, the Fc region comprises one or more mutations. In an embodiment, the Fc region comprises Met-428-Leu and Asn-434-Ser substitutions at residues corresponding to methionine 428 and asparagine 434, each in EU numbering. In an embodiment, the Fc region comprises one, two or three of Met-252-Tyr, Ser-254-Thr and Thr-256-Glu substitutions at residues corresponding to methionine 252, serine 254 and threonine 256, respectively, each in EU numbering.

In an aspect, the disclosure features an antibody molecule, which:

a) competes for binding to C5 with an anti-C5 antibody molecule comprising the heavy chain complementary determining regions (HCDR1, HCDR2 and HCDR3) and the light chain complementary determining regions (LCDR1, LCDR2 and LCDR3) of any of monoclonal antibodies ATG-001, ATG-002, ATG-003, ATG-004, ATG-005, ATG-006, ATG-007, ATG-008, ATG-012, or ATG-013, e.g., as described in Table 1; or

b) binds, or substantially binds, to an epitope that completely or partially overlaps with the epitope of an anti-C5 antibody molecule comprising the heavy chain complementary determining regions (HCDR1, HCDR2 and HCDR3) and the light chain complementary determining regions (LCDR1, LCDR2 and LCDR3) of any of monoclonal antibodies ATG-001 (e.g., SEQ ID NOS: 19, 28, 35, 38, 43, and/or 48, respectively, according to Chothia numbering or SEQ ID NOS: 54, 59, 66, 71, 76, and/or 81, respectively, according to Kabat numbering), ATG-002 (e.g., SEQ ID NOS: 20, 28, 35, 38, 43, and/or 48, respectively, according to Chothia numbering or SEQ ID NOS: 55, 59, 66, 71, 76, and/or 81, respectively, according to Kabat numbering), ATG-003 (e.g., SEQ ID NOS: 21, 29, 35, 38, 43, and/or 48, respectively, according to Chothia numbering or SEQ ID NOS: 54, 60, 66, 71, 76, and/or 81, respectively, according to Kabat numbering), ATG-004 (e.g., SEQ ID NOS: 22, 29, 35, 38, 43, and/or 48, respectively, according to Chothia numbering or SEQ ID NOS: 55, 60, 66, 71, 76, and/or 81, respectively, according to Kabat numbering), ATG-005 (e.g., SEQ ID NOS: 19, 28, 35, 38, 44, and/or 49, respectively, according to Chothia numbering or SEQ ID NOS: 54, 59, 66, 71, 77, and/or 82, respectively, according to Kabat numbering), ATG-006 (e.g., SEQ ID NOS: 20, 28, 35, 38, 44, and/or 49, respectively, according to Chothia numbering or SEQ ID NOS: 55, 59, 66, 71, 77, and/or 82, respectively, according to Kabat numbering), ATG-007 (e.g., SEQ ID NOS: 21, 29, 35, 38, 44, and/or 49, respectively, according to Chothia numbering or SEQ ID NOS: 54, 60, 66, 71, 77, and/or 82, respectively, according to Kabat numbering), ATG-008 (e.g., SEQ ID NOS: 22, 29, 35, 38, 44, and/or 49, respectively, according to Chothia numbering or SEQ ID NOS: 55, 60, 66, 71, 77, and/or 82, respectively, according to Kabat numbering), ATG-012 (e.g., SEQ ID NOS: 22, 29, 35, 38, 44, and/or 49, respectively, according to Chothia numbering or SEQ ID NOS: 55, 60, 66, 71, 77, and/or 82, respectively, according to Kabat numbering), or ATG-013 (e.g., SEQ ID NOS: 22, 29, 35, 38, 44, and/or 49, respectively, according to Chothia numbering or SEQ ID NOS: 55, 60, 66, 71, 77, and/or 82, respectively, according to Kabat numbering), e.g., as described in Table 1.

In an embodiment, the antibody molecule competes for binding with an anti-C5 antibody molecule that comprises a VH and a VL of any of monoclonal antibodies ATG-001, ATG-002, ATG-003, ATG-004, ATG-005, ATG-006, ATG-007, ATG-008, ATG-012, or ATG-013.

In an embodiment, the antibody molecule binds, or substantially binds, to an epitope that completely or partially overlaps with the epitope of an anti-C5 antibody molecule that comprises a VH and a VL of any of monoclonal antibodies ATG-001, ATG-002, ATG-003, ATG-004, ATG-005, ATG-006, ATG-007, ATG-008, ATG-012, or ATG-013.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In an embodiment, the antibody molecule competes for binding with two, three, four, five, six, seven, or all of the anti-C5 antibody molecules that comprise the HCDR1, HCDR2, HCDR3, LCDR1, LCDR2, and LCDR3 of any of monoclonal antibodies ATG-001, ATG-002, ATG-003, ATG-004, ATG-005, ATG-006, ATG-007, ATG-008, ATG-012, or ATG-013.

In an embodiment, the antibody molecule competes for binding with two, three, four, five, six, seven, or all of the anti-C5 antibody molecules that comprises a VH and a VL of any of monoclonal antibodies ATG-001, ATG-002, ATG-003, ATG-004, ATG-005, ATG-006, ATG-007, ATG-008, ATG-012, or ATG-013.

In an embodiment, the antibody molecule binds, or substantially binds, to an epitope that completely or partially overlaps with the epitopes of two, three, four, five, six, seven, or all of the anti-C5 antibody molecules that comprise the HCDR1, HCDR2, HCDR3, LCDR1, LCDR2, and LCDR3 of any of monoclonal antibodies ATG-001, ATG-002, ATG-003, ATG-004, ATG-005, ATG-006, ATG-007, ATG-008, ATG-012, or ATG-013.

In an embodiment, the antibody molecule binds, or substantially binds, to an epitope that completely or partially overlaps with the epitopes of two, three, four, five, six, seven, or all of the anti-C5 antibody molecules that comprises a VH and a VL of any of monoclonal antibodies ATG-001, ATG-002, ATG-003, ATG-004, ATG-005, ATG-006, ATG-007, ATG-008, ATG-012, or ATG-013.

In an embodiment, the antibody molecule binds, or substantially binds, to an epitope that completely or partially overlaps with the epitopes of two, three, four, five, six, seven, or all of the antibody molecules that comprise the HCDR1, HCDR2, HCDR3, LCDR1, LCDR2, and LCDR3 of any of monoclonal antibodies ATG-001, ATG-002, ATG-003, ATG-004, ATG-005, ATG-006, ATG-007, ATG-008, ATG-012, or ATG-013.

In an embodiment, the antibody molecule that comprises the HCDR1, HCDR2, HCDR3, LCDR1, LCDR2, and LCDR3 of any of monoclonal antibodies ATG-001, ATG-002, ATG-003, ATG-004, ATG-005, ATG-006, ATG-007, ATG-008, ATG-012, or ATG-013.

In an embodiment, the antibody molecule binds, or substantially binds, to an epitope that completely or partially overlaps with the epitopes of two, three, four, five, six, seven, or all of the antibody molecules that comprises a VH and a VL of any of monoclonal antibodies ATG-001, ATG-002, ATG-003, ATG-004, ATG-005, ATG-006, ATG-007, ATG-008, ATG-012, or ATG-013.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In an embodiment, the antibody molecule comprises two heavy chain variable regions and two light chain variable regions. In an embodiment, the antibody molecule comprises an antigen-binding fragment. In an embodiment, the antibody molecule comprises a Fab, F(ab′)2, Fv, scFv, or Fd.

In an embodiment, the antibody molecule is an IgG antibody molecule, e.g., comprising a heavy chain constant region of IgG, e.g., chosen from IgG1, IgG2, IgG3, or IgG4, e.g., IgG2 or IgG4. In an embodiment, the antibody molecule is an IgG1 antibody molecule, e.g., having an IgG1 constant region described herein. In another embodiment, the antibody molecule is an IgG2 antibody molecule e.g., having an IgG2 constant region described herein. In an embodiment, the antibody molecule is an IgG3 antibody molecule, e.g., having an IgG3 constant region described herein. In another embodiment, the antibody molecule is an IgG4 antibody molecule e.g., having an IgG4 constant region described herein. In another embodiment, the antibody molecule has a chimeric constant region comprising of IgG2, IgG3 and/or IgG4 isotypes. In an embodiment, the heavy chain constant region comprises one or more amino acid modifications in the hinge, CH2 or CH3 region. In an embodiment, the antibody molecule comprises a light chain constant region of kappa or lambda light chain.

In an embodiment, the antibody molecule comprises an Fc region. In an embodiment, the Fc region comprises one or more mutations. In an embodiment, the Fc region comprises Met-428-Leu and Asn-434-Ser substitutions at residues corresponding to methionine 428 and asparagine 434, each in EU numbering. In an embodiment, the Fc region comprises one, two or three of Met-252-Tyr, Ser-254-Thr and Thr-256-Glu substitutions at residues corresponding to methionine 252, serine 254 and threonine 256, respectively, each in EU numbering.

In an aspect, the disclosure features a composition, e.g., pharmaceutical composition, comprising an antibody molecule described herein. In an embodiment, the composition further comprises a pharmaceutical acceptable carrier.

In an aspect, the disclosure features a nucleic acid molecule encoding a heavy chain variable region (VH), a light chain variable region (VL), or both, of an antibody molecule described herein.

In an aspect, the disclosure features a vector comprising a nucleic acid molecule described herein.

In an aspect, the disclosure features a cell, e.g., an isolated cell, comprising a nucleic acid molecule described herein or a vector described herein.

In an aspect, the disclosure features a kit comprising an antibody molecule described herein and instructions to use of the antibody molecule.

In an aspect, the disclosure features a container comprising an antibody molecule described herein.

In an aspect, the disclosure features a method of producing an anti-C5 antibody molecule, the method comprising culturing a cell described herein under conditions that allow production of an antibody molecule, thereby producing the antibody molecule.

In an embodiment, the method further comprises isolating the antibody molecule.

In an aspect, the disclosure features a method of treating a complement-associated disorder, e.g., a complement-associated disorder described herein, the method comprising administering to a subject in need thereof an effective amount of an antibody molecule described herein or a composition described herein, thereby treating the complement-associated disorder.

In an embodiment, the complement-associated disorder is an inflammation. In an embodiment, the complement-associated disorder is chosen from the group consisting of ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, and thrombotic microangiopathy (TMA). In an embodiment, the complement-associated disorder is PNH. In an embodiment, the complement-associated disorder is aHUS

In an embodiment, the subject is a human. In an embodiment, the antibody molecule is administered to the subject intravenously.

In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg.

In an embodiment, the antibody molecule is administered once a week, twice a week, once every two weeks, once every three weeks, once every four weeks, once every eight weeks, once a month, once every two months, or once every three months.

In an embodiment, administration of the antibody molecule reduces an activity of C5 in a tissue, e.g., in the blood or in blood cells (e.g., red blood cells).

In an embodiment, the method further comprises administering to the subject a second therapy for the complement-associated disorder.

In an aspect, the disclosure features a method of reducing an activity of C5 in a cell or subject, the method comprising contacting the cell or subject, or administering to a subject in need thereof an effective amount of, an antibody molecule described herein or a composition described herein, thereby reducing the activity of C5.

In an embodiment, the cell is a human cell. In an embodiment, the subject is a human.

In an embodiment, the contacting step occurs in vitro, ex vivo, or in vivo. In an embodiment, the antibody molecule is administered to the subject intravenously.

In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg.

In an embodiment, the antibody molecule is administered once a week, twice a week, once every two weeks, once every three weeks, once every four weeks, once every eight weeks, once a month, once every two months, once every three months.

In an embodiment, administration of the antibody molecule reduces the activity of C5 in a tissue, e.g., in the blood or in blood cells (e.g., red blood cells).

In an aspect, the disclosure features use of an antibody molecule described herein or a composition described herein in the treatment, or in the manufacture of a medicament for the treatment, of a disorder described herein.

In another aspect, the disclosure features an antibody molecule described herein or a composition described herein for use in the treatment of a disorder described herein.

In an aspect, the disclosure features a method of detecting a C5 molecule, the method comprising contacting a cell or a sample from a subject with an antibody molecule described herein, thereby detecting the C5 molecule.

In an embodiment, the antibody molecule is coupled with a detectable label. In an embodiment, the C5 molecule is detected in vitro or ex vivo. In another embodiment, the C5 molecule is detected in vivo.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIGS. 1A-1B are a series of diagrams showing an assessment of the purity and integrity of indicated exemplary anti-C5 monoclonal antibodies using (FIG. 1A) SDS-PAGE, under non-reducing or reducing conditions, and (FIG. 1B) size exclusion chromatography (SEC). ATG-001, ATG-002, ATG-003, ATG-005, ATG-006, ATG-007 comprise their respective VH and VL sequences as listed in Table 1 combined with the IgG-1 heavy chain constant region sequence; ATG-004 and ATG-008 comprise their respective VH and VL sequences as listed in Table 1 combined with the IgG2/4-LS heavy chain constant region sequence; and ATG-012 and ATG-013 comprise their respective VH and VL sequences as listed in Table 1 combined with the IgG2/4-YTE heavy chain constant region sequence.

FIGS. 2A-2B are a series of diagrams showing functional assessment of engineered anti-C5 monoclonal antibodies. FIG. 2A shows the binding of the indicated exemplary anti-C5 monoclonal antibodies to human C5. FIG. 2B shows the inhibition of cRBC hemolysis by the indicated exemplary anti-C5 monoclonal antibodies. ATG-001, ATG-002, ATG-003, ATG-005, ATG-006, ATG-007 comprise their respective VH and VL sequences as listed in Table 1 combined with the IgG-1 heavy chain constant region sequence; ATG-004 and ATG-008 comprise their respective VH and VL sequences as listed in Table 1 combined with the IgG2/4-LS heavy chain constant region sequence; and ATG-012 comprises their respective VH and VL sequences as listed in Table 1 combined with the IgG2/4-YTE heavy chain constant region sequence.

### DETAILED DESCRIPTION

Disclosed herein are antibody molecules that bind to C5, e.g., human C5, with high affinity and specificity. Advantageously, several of the antibody molecules describe herein have improved ability to reduce (e.g., inhibit, block, or neutralize) one or more biological activities of C5. Nucleic acid molecules encoding the antibody molecules, expression vectors, host cells, compositions (e.g., pharmaceutical compositions), kits, and methods for making the antibody molecules, are also provided. The antibody molecules and pharmaceutical compositions disclosed herein can be used (alone or in combination with other agents or therapeutic modalities) to treat, prevent and/or diagnose disorders and conditions, e.g., disorders and conditions associated with activation of C5.

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% (e.g., within 4%, 3%, 2%, or 1%) of a given value or range of values.

The compositions and methods disclosed herein encompass polypeptides and nucleic acids having the sequences specified, or sequences substantially identical or similar thereto, e.g., sequences at least 80%, 85%, 90%, 91%, 92%, 93%, 94%, 95%, 96%, 97%, 98% or 99% identical or higher to the sequence specified.

In the context of an amino acid sequence, the term “substantially identical” is used herein to refer to a first amino acid that contains a sufficient or minimum number of amino acid residues that are a) identical to, orb) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In an embodiment, the percent identify between two amino acid or nucleotide sequences is determined using Clustal Omega (Sievers et al. Mol Syst Biol. 2011; 7:539). In an embodiment, the percent identify between two amino acid or nucleotide sequences is determined using Kalign2 (Lassmann et al. Nucleic Acids Res. 2009; 37(3):858-65; Lassmann and Sonnhammer BMC Bioinformatics. 2005; 6:298). In an embodiment, the percent identify between two amino acid or nucleotide sequences is determined using MAFFT (Katoh and Standley Mol Biol Evol. 2013; 30(4):772-80). In an embodiment, the percent identify between two amino acid or nucleotide sequences is determined using MUSCLE (Edgar Nucleic Acids Res. 2004; 32(5):1792-7; Edgar BMC Bioinformatics. 2004; 5:113). In an embodiment, the percent identify between two amino acid or nucleotide sequences is determined using MView (Brown et al. Bioinformatics. 1998; 14(4): 380-1). Other methods for determining the percent identify between two sequences are also described, e.g., in Li et al. Nucleic Acids Res. 2015; 43(W1):W580-4; McWilliam et al. Nucleic Acids Res. 2013; 41(Web Server issue):W597-600.

In an embodiment, the percent identity between two amino acid sequences is determined using the Needleman and Wunsch (J Mol Biol. 1970; 48(3):443-53) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In an embodiment, the percent identity between two nucleotide sequences is determined using the GAP program in the GCG software package (available at www.gcg.com), using an NWSgapdna. CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller (Comput Appl Biosci. 1988; 4(1):11-7) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases, for example, to identify other family members or related sequences. Such searches can be performed using the NBLAST and XBLAST programs (version 2.0) of Altschul, et al. 1990; J Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., Nucleic Acids Res. 1997; 25:3389-3402. When utilizing BLAST and gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions 4) are suitable conditions and the ones that should be used unless otherwise specified.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat,” e.g., a complement-associated disorder, means that a subject (e.g., a human) who has a disorder, e.g., a complement-associated disorder, and/or experiences a symptom of a disorder, e.g., a complement-associated disorder, will, in an embodiment, suffer less a severe symptom and/or recover faster when an antibody molecule is administered than if the antibody molecule were never administered. In an embodiment, when a complement-associated disorder, is treated, the level of C5a and/or C5b may be lower in a treated subject compared to a comparable untreated subject. For example, a diagnostic assay using immunofluorescence or electron microscopy will detect C5a and/or C5b in a biological sample of a subject after administration of an antibody molecule described herein for the effective treatment of the inflammatory disorder. Other assays, e.g., urine tests, blood tests, ultrasound, X-rays, or cystoscopy, can also be used to monitor treatment in a patient, or to detect the presence, e.g., decreased presence (or absence), of a symptom of the disorder, e.g., the complement-associated disorder, after treatment of the disorder in the subject. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., a complement-associated disorder. In an embodiment, treatment is of a subject who does not exhibit certain signs of a disorder, e.g., a complement-associated disorder, and/or of a subject who exhibits only early signs of a disorder, e.g., a complement-associated disorder. In an embodiment, treatment is of a subject who exhibits one or more established signs of a disorder, e.g., a complement-associated disorder. In an embodiment, treatment is of a subject diagnosed as suffering from a disorder, e.g., a complement-associated disorder. In an embodiment, the disorder is a complement-associated disorder described herein.

As used herein, the term “prevent,” a disorder, e.g., a complement-associated disorder, means that a subject (e.g., a human) is less likely to have the disorder, e.g., a complement-associated disorder, if the subject receives the antibody molecule. In an embodiment, the subject is at risk of developing the disorder, e.g., a complement-associated disorder. In an embodiment, the disorder is a complement-associated disorder described herein.

Various aspects of the compositions and methods herein are described in further detail below. Additional definitions are set out throughout the specification.

### Complement Component 5

Complement component 5, also known as C5, C5D, C5a, C5b, CPAMD4, ECLZB, or complement C5, is a protein that in humans is encoded by the C5 gene. C5 is the fifth component of the complement system, which plays a role in inflammatory and cell killing processes. C5 is composed of alpha and beta polypeptide chains that are linked by a disulfide bridge. C5 is cleaved into C5a and C5b. An activation peptide, C5a, which is an anaphylatoxin that possesses spasmogenic and chemotactic activity, is derived from the alpha polypeptide via cleavage with a C5-convertase. The C5b macromolecular cleavage product can form a complex with the C6 complement component, and this complex is the basis for formation of the membrane attack complex, which includes additional complement components.

Exemplary complement pathways, including types, functions and regulation, are described, e.g., by Srijana Khanal at microbeonline.com/complement-system-pathways-functions-regulation/on Nov. 5, 2017.

Exemplary amino acid and nucleotide sequences of human C5 are described, e.g., in SEQ ID NO: 53.

The amino acid sequence of human C5 is provided as follows.

Human C5
(SEQ ID NO: 53)
MGLLGILCFLIFLGKTWGQEQTYVISAPKIFRVGASENIVIQVYGYTEA
FDATISIKSYPDKKFSYSSGHVHLSSENKFQNSAILTIQPKQLPGGQNP
VSYVYLEVVSKHFSKSKRMPITYDNGFLFIHTDKPVYTPDQSVKVRVYS
LNDDLKPAKRETVLTFIDPEGSEVDMVEEIDHIGIISFPDFKIPSNPRY
GMWTIKAKYKEDFSTTGTAYFEVKEYVLPHFSVSIEPEYNFIGYKNFKN
FEITIKARYFYNKVVTEADVYITFGIREDLKDDQKEMMQTAMQNTMLIN
GIAQVTFDSETAVKELSYYSLEDLNNKYLYIAVTVIESTGGFSEEAEIP
GIKYVLSPYKLNLVATPLFLKPGIPYPIKVQVKDSLDQLVGGVPVTLNA
QTIDVNQETSDLDPSKSVTRVDDGVASFVLNLPSGVTVLEFNVKTDAPD
LPEENQAREGYRAIAYSSLSQSYLYIDWTDNHKALLVGEHLNIIVTPKS
PYIDKITHYNYLILSKGKIIHFGTREKFSDASYQSINIPVTQNMVPSSR
LLVYYIVTGEQTAELVSDSVWLNIEEKCGNQLQVHLSPDADAYSPGQTV
SLNMATGMDSWVALAAVDSAVYGVQRGAKKPLERVFQFLEKSDLGCGAG
GGLNNANVFHLAGLTFLTNANADDSQENDEPCKEILRPRRTLQKKIEEI
AAKYKHSVVKKCCYDGACVNNDETCEQRAARISLGPRCIKAFTECCVVA
SQLRANISHKDMQLGRLHMKTLLPVSKPEIRSYFPESWLWEVHLVPRRK
QLQFALPDSLTTWEIQGVGISNTGICVADTVKAKVFKDVFLEMNIPYSV
VRGEQIQLKGTVYNYRTSGMQFCVKMSAVEGICTSESPVIDHQGTKSSK
CVRQKVEGSSSHLVTFTVLPLEIGLHNINFSLETWFGKEILVKTLRVVP
EGVKRESYSGVTLDPRGIYGTISRRKEFPYRIPLDLVPKTEIKRILSVK
GLLVGEILSAVLSQEGINILTHLPKGSAEAELMSVVPVFYVFHYLETGN
HWNIFHSDPLIEKQKLKKKLKEGMLSIMSYRNADYSYSVWKGGSASTWL
TAFALRVLGQVNKYVEQNQNSICNSLLWLVENYQLDNGSFKENSQYQPI
KLQGTLPVEARENSLYLTAFTVIGIRKAFDICPLVKIDIALIKADNFLL
ENTLPAQSTFTLAISAYALSLGDKTHPQFRSIVSALKREALVKGNPPIY
RFWKDNLQHKDSSVPNTGTARMVETTAYALLTSLNLKDINYVNPVIKWL
SEEQRYGGGFYSTQDTINAIEGLIEYSLLVKQLRLSMDIDVSYKHKGAL
HNYKMTDKNFLGRPVEVLLNDDLIVSTGFGSGLATVHVTIVVHKTSTSE
EVCSFYLKIDTQDIEASHYRGYGNSDYKRIVACASYKPSREESSSGSSH
AVMDISLPTGISANEEDLKALVEGVDQLFTDYQIKDGHVILQLNSIPSS
DFLCVRFRIFELFEVGFLSPATFTVYEYHRPDKQCTMFYSTSNIKIQKV
CEGAACKCVEADCGQMQEELDLTISAETRKQTACKPEIAYAYKVSITSI
TVENVFVKYKATLLDIYKTGEAVAEKDSEITFIKKVICTNAELVKGRQY
LIMGKEALQIKYNFSFRYIYPLDSLTTNIEYTNPRDTTCSSCQAFLANL
DEFAEDIFLNGC

Other variant and alternative sequences of human C5 are provided, e.g., as Genbank Accession Nos. NP_001304092.1 (isoform 2), NP_001304093.1 (isoform 3 precursor), and NP_001726.2 (isoform 1 preproprotein), as listed as of Jun. 11, 2018. The respective nucleotide sequences encoding the above-listed human C5 sequences are provided as Genbank Accession Nos. NM_001317163.1, NM_001317164.1, and NM_001735.2, as listed as of Jun. 11, 2018.

Exemplary amino acid and nucleotide sequences of mouse C5 (also referred to as hemolytic complement, Hc, He, C5a, or Hfib2) are described, e.g., in Genbank Accession Nos. NP_034536.2 and NM_010406.2, respectively, as listed as of Jun. 11, 2018.

The amino acid sequence of mouseC5 is provided as follows.

SEQ ID NO: 93
MPLPAMGLWGILCLLIFLDKTWGQEQTYVISAPKILRVGSSENVVIQVH
GYTEAFDATLSLKSYPDKKVTFSSGYVNLSPENKFQNAALLTLQPNQVP
REESPVSHVYLEVVSKHFSKSKKIPITYNNGILFIHTDKPVYTPDQSVK
IRVYSLGDDLKPAKRETVLTFIDPEGSEVDIVEENDYTGIISFPDFKIP
SNPKYGVWTIKANYKKDFITTGTAYFEIKEYVLPRFSVSIELERTFIGY
KNFKNFEITVKARYFYNKVVPDAEVYAFFGLREDIKDEEKQMMHKATQA
AKLVDGVAQISFDSETAVKELSYNSLEDLNNKYLYIAVTVTESSGGFSE
EAEIPGVKYVLSPYTLNLVATPLFVKPGIPFSIKAQVKDSLEQAVGGVP
VTLMAQTVDVNQETSDLETKRSITHDTDGVAVFVLNLPSNVTVLKFEIR
TDDPELPEENQASKEYEAVAYSSLSQSYIYIAWTENYKPMLVGEYLNIM
VTPKSPYIDKITHYNYLILSKGKIVQYGTREKLFSSTYQNINIPVTQNM
VPSARLLVYYIVTGEQTAELVADAVWINIEEKCGNQLQVHLSPDEYVYS
PGQTVSLDMVTEADSWVALSAVDRAVYKVQGNAKRAMQRVFQALDEKSD
LGCGAGGGHDNADVFHLAGLTFLTNANADDSHYRDDSCKEILRSKRNLH
LLRQKIEEQAAKYKHSVPKKCCYDGARVNFYETCEERVARVTIGPLCIR
AFNECCTIANKIRKESPHKPVQLGRIHIKTLLPVMKADIRSYFPESWLW
EIHRVPKRKQLQVTLPDSLTTWEIQGIGISDNGICVADTLKAKVFKEVF
LEMNIPYSVVRGEQIQLKGTVYNYMTSGTKFCVKMSAVEGICTSGSSAA
SLHTSRPSRCVFQRIEGSSSHLVTFILLPLEIGLHSINFSLETSFGKDI
LVKTLRVVPEGVKRESYAGVILDPKGIRGIVNRRKEFPYRIPLDLVPKT
KVERILSVKGLLVGEFLSTVLSKEGINILTHLPKGSAEAELMSIAPVFY
VFHYLEAGNHWNIFYPDTLSKRQSLEKKIKQGVVSVMSYRNADYSYSMW
KGASASTWLTAFALRVLGQVAKYVKQDENSICNSLLWLVEKCQLENGSF
KENSQYLPIKLQGTLPAEAQEKTLYLTAFSVIGIRKAVDICPTMKIHTA
LDKADSFLLENTLPSKSTFTLAIVAYALSLGDRTHPRFRLIVSALRKEA
FVKGDPPIYRYWRDTLKRPDSSVPSSGTAGMVETTAYALLASLKLKDMN
YANPIIKWLSEEQRYGGGFYSTQDTINAIEGLTEYSLLLKQIHLDMDIN
VAYKHEGDFHKYKVTEKHFLGRPVEVSLNDDLVVSTGYSSGLATVYVKT
VVHKISVSEEFCSFYLKIDTQDIEASSHFRLSDSGFKRIIACASYKPSK
EESTSGSSHAVMDISLPTGIGANEEDLRALVEGVDQLLTDYQIKDGHVI
LQLNSIPSRDFLCVRFRIFELFQVGFLNPATFTVYEYHRPDKQCTMIYS
ISDTRLQKVCEGAACTCVEADCAQLQAEVDLAISADSRKEKACKPETAY
AYKVRITSATEENVFVKYTAILLVTYKTGEAADENSEVTFIKKMSCINA
NLVKGKQYLIMGKEVLQIKHNFSFKYIYPLDSSTWIEYWPTDTTCPSCQ
AFVENLNNFAEDLFLNSCE

In an embodiment, when an anti-C5 antibody molecule binds, or substantially binds, to C5, it binds, or substantially binds, to one or more isoforms of C5, e.g., one or more isoforms of human C5 described herein. In an embodiment, the antibody molecule binds or substantially binds to C5 having the amino acid sequence of SEQ ID NO: 53.

### Antibody Molecules

Disclosed herein are antibody molecules that bind to C5, e.g., an anti-C5 antibody molecule described herein.

As used herein, the term “antibody molecule” refers to a protein, e.g., an immunoglobulin chain or a fragment thereof, comprising at least one immunoglobulin variable domain sequence. The term “antibody molecule” includes, for example, a full-length antibody and an antigen-binding fragment of an antibody.

For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv or sc(Fv)2, for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. In embodiments, the antibody molecule is a whole IgG antibody. The antibody molecule can also be a human, humanized, CDR-grafted, or in vitro generated antibody. The antibody molecule can have a heavy chain constant region chosen from, e.g., IgG1, IgG2, IgG3, IgG4, or a chimera of two or more isotypes. The antibody molecule can also have a light chain chosen from, e.g., kappa or lambda. The term “immunoglobulin” (Ig) is used interchangeably with the term “antibody” herein. In embodiments, the antibody molecule is a multispecific antibody molecule (e.g., a bispecific antibody molecule).

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

In an embodiment, the antibody molecule is a single chain antibody. A single-chain antibody (scFv) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In an embodiment, the antibody molecule is a single domain antibody. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In an embodiment, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also within the scope of the invention.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3). As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242 (“Kabat” numbering scheme); Chothia, C. et al. (1987)J Mol. Biol. 196:901-917 (“Chothia” numbering scheme); and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). As used herein, the CDRs defined according the “Chothia” number scheme are also sometimes referred to as “hypervariable loops.” Under all definitions, each VH and VL typically includes three CDRs and four FRs, arranged from amino-terminus to carboxy-terminus in the following order: FR1, CDR1, FR2, CDR2, FR3, CDR3, FR4.

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the anti-C5 antibody molecules described herein can include any combination of one or more Kabat CDRs and/or Chothia hypervariable loops, e.g., described in Table 1.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody molecule that comprises determinants that form an interface that binds to an antigen, e.g., C5, e.g., human C5, or an epitope thereof. With respect to proteins (or protein mimetics), the antigen-binding region typically includes one or more loops (of at least, e.g., four amino acids or amino acid mimics) that form an interface that binds to the antigen, e.g., C5, e.g., human C5. Typically, the antigen-binding region of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-C5 antibody molecule, e.g., an anti-C5 antibody molecule provided herein, to a target, e.g., C5, e.g., human C5. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA, or a BIACORE assay. In an embodiment, a competition binding assay is a quantitative competition assay. In an embodiment, a first anti-C5 antibody molecule is said to compete for binding to the target with a second anti-C5 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum and potential allergic reactions (see, e.g., Saleh et al., Cancer Immunol. Immunother., 32:180-190 (1990); LoBuglio et al., Hybridoma, 5:5117-5123 (1986)).

The antibody molecule can be a polyclonal or a monoclonal antibody. In an embodiment, the antibody can be recombinantly produced, e.g., produced by any suitable phage display or combinatorial methods.

Various phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In an embodiment, the antibody molecule is a fully human antibody (e.g., an antibody made in a mouse which has been genetically engineered to produce an antibody from a human immunoglobulin sequence), or a non-human antibody, e.g., a rodent (e.g., mouse or rat), goat, primate (e.g., monkey), camel antibody. In an embodiment, the non-human antibody is a rodent (e.g., mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by any suitable recombinant DNA technique. Several are known in the art (see Robinson et al., International Patent Application Publication No. WO1987/002671; Akira, et al., European Patent Application Publication No. 184,187; Taniguchi, M., European Patent Application Publication No. 171,496; Morrison et al., European Patent Application Publication No. 173,494; Neuberger et al., International Patent Application Publication No. WO 86/01533; Cabilly et al. U.S. Pat. No. 4,816,567; Cabilly et al., European Patent Application Publication No. 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In an embodiment, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In an embodiment, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by any suitable method, and several such methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies (UK Patent Application GB 2188638A, filed on Mar. 26, 1987; Winter U.S. Pat. No. 5,225,539), the contents of which is expressly incorporated by reference.

Also provided are humanized antibodies in which specific amino acids have been substituted, deleted or added. Criteria for selecting amino acids from the donor are described in, e.g., U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In an embodiment, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody molecule (e.g., to increase or decrease one or more of: Fc receptor binding, antibody glycosylation, the number of cysteine residues, effector cell function, and/or complement function). In an embodiment, the antibody molecule has effector function and can fix complement. In another embodiment, the antibody molecule does not recruit effector cells or fix complement. In an embodiment, the antibody molecule has reduced or no ability to bind an Fc receptor. For example, it may be an isotype or subtype, fragment or other mutant, which does not support binding to an Fc receptor, e.g., it has a mutated or deleted Fc receptor binding region.

In an embodiment, a constant region of the antibody molecule is altered. Methods for altering an antibody constant region are known in the art. Antibody molecules s with altered function, e.g. altered affinity for an effector ligand, such as FcR on a cell, or the Cl component of complement can be produced by replacing at least one amino acid residue in the constant portion of the antibody with a different residue (see, e.g., EP 388,151 A1, U.S. Pat. Nos. 5,624,821 and 5,648,260, the contents of all of which are hereby incorporated by reference). Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

In an embodiment, the only amino acids in the antibody molecule are canonical amino acids. In an embodiment, the antibody molecule comprises naturally-occurring amino acids; analogs, derivatives and congeners thereof; amino acid analogs having variant side chains; and/or all stereoisomers of any of any of the foregoing. The antibody molecule may comprise the D- or L-optical isomers of amino acids and peptidomimetics.

In an embodiment, the antibody molecule comprises a monoclonal antibody (e.g., a full length antibody which has an immunoglobulin Fc region). In an embodiment, the antibody molecule comprises a full length antibody or full length immunoglobulin chain. In an embodiment, the antibody molecule comprises an antigen binding or functional fragment of a full length antibody or full length immunoglobulin chain.

In an embodiment, the antibody molecule is a monospecific antibody molecule, e.g., it binds a single epitope. For example, a monospecific antibody molecule can have a plurality of immunoglobulin variable region sequences, each of which binds the same epitope.

In an embodiment, the antibody molecule is a multispecific antibody molecule, e.g., it comprises a plurality of immunoglobulin variable region sequences, wherein a first immunoglobulin variable region sequence of the plurality has binding specificity for a first epitope and a second immunoglobulin variable region sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, a multispecific antibody molecule is a bispecific antibody molecule. A bispecific antibody has specificity for no more than two antigens. A bispecific antibody molecule is typically characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment, the first and second epitopes do not overlap. In an embodiment, the first and second epitopes are on different antigens, e.g., the different proteins (or different subunits of a multimeric protein). In an embodiment, a bispecific antibody molecule comprises a heavy chain variable region sequence and a light chain variable region sequence which have binding specificity for a first epitope, and a heavy chain variable region sequence and a light chain variable region sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or a fragment thereof, having binding specificity for a first epitope, and a half antibody, or fragment thereof, having binding specificity for a second epitope. In an embodiment a bispecific antibody molecule comprises an scFv, or a fragment thereof, have binding specificity for a first epitope, and an scFv, or a fragment thereof, have binding specificity for a second epitope.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094;

String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through noncovalent or chemical crosslinking to form, e.g., homobivalent, heterobivalent, trivalent, and tetravalent structures using both scFV or diabody type format, as described in, e.g., U.S. Pat. No. 5,869,620. The contents of the above-referenced applications are incorporated herein by reference in their entirety.

Additional methods of making multispecific or bispecific antibody molecules can be found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587, US2002/076406, US2002/103345, US2003/207346, US2003/211078, US2004/219643, US2004/220388, US2004/242847, US2005/003403, US2005/004352, US2005/069552, US2005/079170, US2005/100543, US2005/136049, US2005/136051, US2005/163782, US2005/266425, US2006/083747, US2006/120960, US2006/204493, US2006/263367, US2007/004909, US2007/087381, US2007/128150, US2007/141049, US2007/154901, US2007/274985, US2008/050370, US2008/069820, US2008/152645, US2008/171855, US2008/241884, US2008/254512, US2008/260738, US2009/130106, US2009/148905, US2009/155275, US2009/162359, US2009/162360, US2009/175851, US2009/175867, US2009/232811, US2009/234105, US2009/263392, US2009/274649, EP346087, WO00/06605, WO02/072635, WO04/081051, WO06/020258, WO2007/044887, WO2007/095338A2, WO2007/137760A2, WO2008/119353, WO2009/021754, WO2009/068630, WO91/03493, WO93/23537, WO94/09131, WO94/12625, WO95/09917, WO96/37621, WO99/64460. The contents of the above-referenced applications are incorporated herein by reference in their entirety.

A polypeptide of an antibody molecule described herein may be linear or branched, it may comprise modified amino acids, and it may be interrupted by non-amino acids. The antibody molecule may also be modified; for example, by disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The antibody molecule described herein can be used alone in unconjugated form, or can be bound to a substance, e.g., a toxin or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-C5 antibody can be coupled to a radioactive isotope such as an α-, β-, or γ-emitter, or a β- and γ-emitter.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a toxin, a pharmaceutical agent, and/or a protein or peptide that can mediate association of the antibody or antibody portion with another molecule (such as a streptavidin core region or a polyhistidine tag).

Some types of derivatized antibody molecule are produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-dengue antibody molecule may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecules can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule described herein can be conjugated to another molecular entity, typically a label or a therapeutic (e.g., antimicrobial (e.g., antibacterial or bactericidal), immunomodulatory, immunostimularoty, cytotoxic, or cytostatic) agent or moiety. Radioactive isotopes can be used in diagnostic or therapeutic applications. Radioactive isotopes that can be coupled to the antibody molecules include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At) rhenium (¹⁸⁶Re), bismuth (²¹²Bi or ²¹³Bi), indium (¹¹¹In) technetium (^{99m} Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At) rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In) technetium (^{99m}Tc), phosphorus (³²P), carbon (¹⁴C), and tritium (³H), or one or more of the therapeutic isotopes listed above.

The present disclosure provides radiolabeled antibody molecules and methods of labeling the same. In an embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

In an embodiment, the antibody molecule is conjugated to a therapeutic agent. Therapeutically active radioisotopes are disclosed herein. Examples of other therapeutic agents include, but are not limited to, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see e.g., U.S. Pat. No. 5,208,020), CC-1065 (see e.g., U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In an embodiment, the anti-C5 antibody molecule (e.g., a monospecific, bispecific, or multispecific antibody molecule) is covalently linked, e.g., fused, to another partner e.g., a protein, e.g., as a fusion molecule (e.g., a fusion protein).

As used herein, a “fusion protein” and “fusion polypeptide” refer to a polypeptide having at least two portions covalently linked together, where each of the portions is a polypeptide. In an embodiment, each of the portions is a polypeptide that has a different property. The property can be a biological property, such as activity in vitro or in vivo. The property can also be simple chemical or physical property, such as binding to a target molecule, catalysis of a reaction, etc. The two portions can be linked directly by a single peptide bond or through a linker (e.g., peptide linker), but are in reading frame with each other.

In one aspect, the invention features a method of providing a target binding agent that specifically binds to C5 (e.g., human C5). For example, the target binding molecule is an antibody molecule. The method includes: providing a target protein that comprises at least a portion of non-human protein, the portion being homologous to (e.g., at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining a binding agent (e.g., an antibody molecule) that specifically binds to the target protein; and evaluating efficacy of the binding agent in modulating an activity of the target protein. The method can further include administering the binding agent (e.g., antibody molecule) or a derivative (e.g., a humanized antibody molecule) to a subject (e.g., a human subject).

In another aspect, this disclosure provides a method of making an antibody molecule disclosed herein. The method includes: providing an antigen, e.g., C5 (e.g., human C5) or a fragment thereof; obtaining an antibody molecule that specifically binds to the antigen; evaluating efficacy of the antibody molecule in modulating activity of the antigen and/or organism expressing the antigen, e.g., C5, e.g., human C5. The method can further include administering the antibody molecule, including a derivative thereof (e.g., a humanized antibody molecule) to a subject, e.g., a human.

This disclosure provides an isolated nucleic acid molecule encoding the above antibody molecule, vectors and host cells thereof. The nucleic acid molecule includes, but is not limited to, RNA, genomic DNA and cDNA.

Amino acid and nucleotide sequences of exemplary antibody molecules are described in Tables 1-5.

TABLE 1
Amino acid sequences of heavy chain variable regions (VHs) and light chain variable regions (VLs)
of exemplary antibody molecules

| | | | SEQ | | | SEQ | | | SEQ |
|---|---|---|---|---|---|---|---|---|---|
| Antibody | Chain | Amino Acid Sequence | ID NO | Chothia CDR | | ID NO | Kabat CDR | | ID NO |
| ATG-001 | VH | QVQLVQSGAEVKKPGASVKVSCK | 1 | HCDR1 | GYIFTNY | 19 | HCDR1 | NYWMQ | 54 |
| | | ASGYIFTNYWMQWVRQAPGQGLE | | HCDR2 | LPGTGS | 28 | HCDR2 | EILPGTGSTEYAQK | 59 |
| | | WMGEILPGTGSTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 10 | LCDR1 | GATENIYGALN | 38 | LCDR1 | GATENIYGALN | 71 |
| | | GATENIYGALNWYQHEPGKAPKL | | LCDR2 | GASNLAD | 43 | LCDR2 | GASNLAD | 76 |
| | | LIYGASNLADGVTSRFSGSGSGT | | LCDR3 | QNVLNTPLS | 48 | LCDR3 | QNVLNTPLS | 81 |
| | | DFTLTISTLQPEDFATYYCQNVL | | | | | | | |
| | | NTPLSFGGGTKVDIK | | | | | | | |
| ATG-002 | VH | QVQLVQSGAEVKKPGASVKVSCK | 2 | HCDR1 | GYIFTDFY | 20 | HCDR1 | DFYMQ | 55 |
| | | ASGYIFTDFYMQWVRQAPGQGLE | | HCDR2 | LPGTGS | 28 | HCDR2 | EILPGTGSTEYAQK | 59 |
| | | WMGEILPGTGSTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 10 | LCDR1 | GATENIYGALN | 38 | LCDR1 | GATENIYGALN | 71 |
| | | GATENIYGALNWYQHEPGKAPKL | | LCDR2 | GASNLAD | 43 | LCDR2 | GASNLAD | 76 |
| | | LIYGASNLADGVTSRFSGSGSGT | | LCDR3 | QNVLNTPLS | 48 | LCDR3 | QNVLNTPLS | 81 |
| | | DFTLTISTLQPEDFATYYCQNVL | | | | | | | |
| | | NTPLSFGGGTKVDIK | | | | | | | |
| ATG-003 | VH | QVQLVQSGAEVKKPGASVKVSCK | 3 | HCDR1 | GHIFTNY | 21 | HCDR1 | NYWMQ | 54 |
| | | ASGHIFTNYWMQWVRQAPGQGLE | | HCDR2 | LPGTGH | 29 | HCDR2 | EILPGTGHTEYAQK | 60 |
| | | WMGEILPGTGHTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 10 | LCDR1 | GATENIYGALN | 38 | LCDR1 | GATENIYGALN | 71 |
| | | GATENIYGALNWYQHEPGKAPKL | | LCDR2 | GASNLAD | 43 | LCDR2 | GASNLAD | 76 |
| | | LIYGASNLADGVTSRFSGSGSGT | | LCDR3 | QNVLNTPLS | 48 | LCDR3 | QNVLNTPLS | 81 |
| | | DFTLTISTLQPEDFATYYCQNVL | | | | | | | |
| | | NTPLSFGGGTKVDIK | | | | | | | |
| ATG-004 | VH | QVQLVQSGAEVKKPGASVKVSCK | 4 | HCDR1 | GHIFTDFY | 22 | HCDR1 | DFYMQ | 55 |
| | | ASGHIFTDFYMQWVRQAPGQGLE | | HCDR2 | LPGTGH | 29 | HCDR2 | EILPGTGHTEYAQK | 60 |
| | | WMGEILPGTGHTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 10 | LCDR1 | GATENIYGALN | 38 | LCDR1 | GATENIYGALN | 71 |
| | | GATENIYGALNWYQHEPGKAPKL | | LCDR2 | GASNLAD | 43 | LCDR2 | GASNLAD | 76 |
| | | LIYGASNLADGVTSRFSGSGSGT | | LCDR3 | QNVLNTPLS | 48 | LCDR3 | QNVLNTPLS | 81 |
| | | DFTLTISTLQPEDFATYYCQNVL | | | | | | | |
| | | NTPLSFGGGTKVDIK | | | | | | | |
| ATG-005 | VH | QVQLVQSGAEVKKPGASVKVSCK | 1 | HCDR1 | GYIFTNY | 19 | HCDR1 | NYWMQ | 54 |
| | | ASGYIFTNYWMQWVRQAPGQGLE | | HCDR2 | LPGTGS | 28 | HCDR2 | EILPGTGSTEYAQK | 59 |
| | | WMGEILPGTGSTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 11 | LCDR1 | GATENIYGALN | 38 | LCDR1 | GATENIYGALN | 71 |
| | | GATENIYGALNWYQHEPGKAPKL | | LCDR2 | GASNRYT | 44 | LCDR2 | GASNRYT | 77 |
| | | LIYGASNRYTGVTSRFSGSGSGT | | LCDR3 | QQVLNTPVS | 49 | LCDR3 | QQVLNTPVS | 82 |
| | | DFTLTISTLQPEDFATYYCQQVL | | | | | | | |
| | | NTPVSFGGGTKVDIK | | | | | | | |
| ATG-006 | VH | QVQLVQSGAEVKKPGASVKVSCK | 2 | HCDR1 | GYIFTDFY | 20 | HCDR1 | DFYMQ | 55 |
| | | ASGYIFTDFYMQWVRQAPGQGLE | | HCDR2 | LPGTGS | 28 | HCDR2 | EILPGTGSTEYAQK | 59 |
| | | WMGEILPGTGSTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 11 | LCDR1 | GATENIYGALN | 38 | LCDR1 | GATENIYGALN | 71 |
| | | GATENIYGALNWYQHEPGKAPKL | | LCDR2 | GASNRYT | 44 | LCDR2 | GASNRYT | 77 |
| | | LIYGASNRYTGVTSRFSGSGSGT | | LCDR3 | QQVLNTPVS | 49 | LCDR3 | QQVLNTPVS | 82 |
| | | DFTLTISTLQPEDFATYYCQQVL | | | | | | | |
| | | NTPVSFGGGTKVDIK | | | | | | | |
| ATG-007 | VH | QVQLVQSGAEVKKPGASVKVSCK | 3 | HCDR1 | GHIFTNY | 21 | HCDR1 | NYWMQ | 54 |
| | | ASGHIFTNYWMQWVRQAPGQGLE | | HCDR2 | LPGTGH | 29 | HCDR2 | EILPGTGHTEYAQK | 60 |
| | | WMGEILPGTGHTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 11 | LCDR1 | GATENIYGALN | 38 | LCDR1 | GATENIYGALN | 71 |
| | | GATENIYGALNWYQHEPGKAPKL | | LCDR2 | GASNRYT | 44 | LCDR2 | GASNRYT | 77 |
| | | LIYGASNRYTGVTSRFSGSGSGT | | LCDR3 | QQVLNTPVS | 49 | LCDR3 | QQVLNTPVS | 82 |
| | | DFTLTISTLQPEDFATYYCQQVL | | | | | | | |
| | | NTPVSFGGGTKVDIK | | | | | | | |
| ATG-008 | VH | QVQLVQSGAEVKKPGASVKVSCK | 4 | HCDR1 | GHIFTDFY | 22 | HCDR1 | DFYMQ | 55 |
| | | ASGHIFTDFYMQWVRQAPGQGLE | | HCDR2 | LPGTGH | 29 | HCDR2 | EILPGTGHTEYAQK | 60 |
| | | WMGEILPGTGHTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 11 | LCDR1 | GATENIYGALN | 38 | LCDR1 | GATENIYGALN | 71 |
| | | GATENIYGALNWYQHEPGKAPKL | | LCDR2 | GASNRYT | 44 | LCDR2 | GASNRYT | 77 |
| | | LIYGASNRYTGVTSRFSGSGSGT | | LCDR3 | QQVLNTPVS | 49 | LCDR3 | QQVLNTPVS | 82 |
| | | DFTLTISTLQPEDFATYYCQQVL | | | | | | | |
| | | NTPVSFGGGTKVDIK | | | | | | | |
| ATG-012 | VH | QVQLVQSGAEVKKPGASVKVSCK | 4 | HCDR1 | GHIFTDFY | 22 | HCDR1 | DFYMQ | 55 |
| | | ASGHIFTDFYMQWVRQAPGQGLE | | HCDR2 | LPGTGH | 29 | HCDR2 | EILPGTGHTEYAQK | 60 |
| | | WMGEILPGTGHTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| | VL | EIVMTQSPSSLSASVGDRVTITC | 11 | LCDR1 | GATENIYGALN | 38 | LCDR1 | GATENIYGALN | 71 |
| | | GATENIYGALNWYQHEPGKAPKL | | LCDR2 | GASNRYT | 44 | LCDR2 | GASNRYT | 77 |
| | | LIYGASNRYTGVTSRFSGSGSGT | | LCDR3 | QQVLNTPVS | 49 | LCDR3 | QQVLNTPVS | 82 |
| | | DFTLTISTLQPEDFATYYCQQVL | | | | | | | |
| | | NTPVSFGGGTKVDIK | | | | | | | |
| ATG-013 | VH | QVQLVQSGAEVKKPGASVKVSCK | 2 | HCDR1 | GYIFTDFY | 20 | HCDR1 | DFYMQ | 55 |
| | | ASGYIFTDFYMQWVRQAPGQGLE | | HCDR2 | LPGTGS | 28 | HCDR2 | EILPGTGSTEYAQK | 59 |
| | | WMGEILPGTGSTEYAQKFQGRVT | | | | | | FQG | |
| | | MTRDTSISTVYMEVRRLRSDDTA | | HCDR3 | YFFGSTPNWYFDV | 35 | HCDR3 | YFFGSTPNWYFDV | 66 |
| | | VYYCARYFFGSTPNWYFDVWGQG | | | | | | | |
| | | TLVTVSS | | | | | | | |
| VL | | EIVMTQSPSSLSASVGDRVTITC | 11 | LCDR1 | GATENIYGALN | 38 | LCDR1 | GATENIYGALN | 71 |
| | | GATENIYGALNWYQHEPGKAPKL | | LCDR2 | GASNRYT | 44 | LCDR2 | GASNRYT | 77 |
| | | LIYGASNRYTGVTSRFSGSGSGT | | LCDR3 | QQVLNTPVS | 49 | LCDR3 | QQVLNTPVS | 82 |
| | | DFTLTISTLQPEDFATYYCQQVL | | | | | | | |
| | | NTPVSFGGGTKVDIK | | | | | | | |

TABLE 2
List of Exemplary VH and VL sequences

| Description | SEQ ID NO: | Amino Acid Sequence |
|---|---|---|
| VH-3 | 1 | QVQLVQSGAEVKKPGASVKVSCKASGYIFTNYWMQWVRQAPGQGLEWMGEILPGTGSTEYAQKFQGRVTM |
| | | TRDTSISTVYMEVRRLRSDDTAVYYCARYFFGSTPNWYFDVWGQGTLVTVSS |
| VH-4 | 2 | QVQLVQSGAEVKKPGASVKVSCKASGYIFTDFYMQWVRQAPGQGLEWMGEILPGTGSTEYAQKFQGRVTM |
| | | TRDTSISTVYMEVRRLRSDDTAVYYCARYFFGSTPNWYFDVWGQGTLVTVSS |
| VH-5 | 3 | QVQLVQSGAEVKKPGASVKVSCKASGHIFTNYWMQWVRQAPGQGLEWMGEILPGTGHTEYAQKFQGRVTM |
| | | TRDTSISTVYMEVRRLRSDDTAVYYCARYFFGSTPNWYFDVWGQGTLVTVSS |
| VH-6 | 4 | QVQLVQSGAEVKKPGASVKVSCKASGHIFTDFYMQWVRQAPGQGLEWMGEILPGTGHTEYAQKFQGRVTM |
| | | TRDTSISTVYMEVRRLRSDDTAVYYCARYFFGSTPNWYFDVWGQGTLVTVSS |
| VH-7 | 5 | QVKLLEQSGAEVKKPGASVKVSCKASGYIFSNYWIQWVRQAPGQRLEWMGEILAGSGSTEYSQKFRGRVT |
| | | FTRDTSATTAYMGLSSLRPEDTAVYYCARYPFGSSPNWEFDYWGQGTLVTVSS |
| VH-8 | 6 | QVKLLEQSGAEVKKPGASVKVSCKASGFIFSNYWIQWVRQAPGQRLEWMGEVLPGSGSTEYSQKFRGRVT |
| | | MTRDTSATTAYMGLSSLRPEDTAVYYCARYYFGSSPNWYFDVWGQGTLVTVSS |
| VH-9 | 7 | QVKLLEQSGAEVKKPGASVKVSCKASGYIFTSYWIQWVRQAPGQRLEWMGEILPGDGSTEYSQKFRGRVT |
| | | MTRDTSATTAYMGLSSLRPEDTAVYYCARYFFGSSPNWAMDYWGQGTLVTVSS |
| VH-10 | 8 | QVKLLEQSGAEVKKPGASVKVSCKASGYIFTSYWIQWVRQAPGQRLEWMGEILPTNGSTEYSQKFRGRVT |
| | | MTRDTSATTAYMGLSSLRPEDTAVYYCARYFFGSSPNWAMDYWGQGTLVTVSS |
| VH-11 | 9 | QVQLVQSGAEVKKPGASVKVSCKASGYTFIGNYMHWVRQAPGQGLEYMGWINPKSGDTNYAQKFQGRVIM |
| | | TRDTSISTVYMEVRRLRSDDTAVYYCATGWWGMDVWGQGTLVTVSS |
| VL-12 | 10 | EIVMTQSPSSLSASVGDRVTITCGATENIYGALNWYQHEPGKAPKLLIYGASNLADGVTSRFSGSGSGTD |
| | | FTLTISTLQPEDFATYYCQNVLNTPLSFGGGTKVDIK |
| VL-13 | 11 | EIVMTQSPSSLSASVGDRVTITCGATENIYGALNWYQHEPGKAPKLLIYGASNRYTGVTSRFSGSGSGTD |
| | | FTLTISTLQPEDFATYYCQQVLNTPVSFGGGTKVDIK |
| VL-14 | 12 | ELVMTQSPSSLSASVGDRVNIACRASEGIYGALAWYQQKPGKAPRLLIYDASNLESGVPSRFSGSGSGTD |
| | | FTLTISSLQPEDFAIYYCQQVLNTPLTFGGGTKVEIK |
| VL-15 | 13 | ELQMTQSPSSLSASVGDRVNIACRASESIYGALNWYQQKPGKAPRLLIYDASNLESGVPSRFSGSGSGTD |
| | | FTLTISSLQPEDFAIYYCQNVLSTPWTFGGGTKVEIK |
| VL-16 | 14 | ELQMTQSPSSLSASVGDRVNIACGASENIYGALHWYQQKPGKAPRLLIYGASTRESGVPSRFSGSGSGTD |
| | | FTLTISSLQPEDFAIYYCQNVLNTPYTFGGGTKVEIK |
| VL-17 | 15 | DIVMTQSPSSLSASVGDRVTITCRASQNINNYLHWYQHEPGKAPKLLIYAASNLQGGVISRFSGSGSGTD |
| | | FTLTISTLQPEDFATYYCLQTHAYPLTFGGGTKVDIK |

TABLE 3
List of Exemplary Chothia CDR sequences

| Description | SEQ ID NO: | Amino Acid Sequence |
|---|---|---|
| HCDR1 | 16 | GYIFSNY |
| Sequences | 17 | GFIFSNY |
| | 18 | GYIFTSY |
| | 19 | GYIFTNY |
| | 20 | GYIFTDFY |
| | 21 | GHIFTNY |
| | 22 | GHIFTDFY |
| | 23 | GYTFTGNY |
| HCDR2 | 24 | LAGSGS |
| Sequences | 25 | LPGSGS |
| | 26 | LPGDGS |
| | 27 | LPTNGS |
| | 28 | LPGTGS |
| | 29 | LPGTGH |
| | 30 | NPKSGD |
| HCDR3 | 31 | YPFGSSPNWEFDY |
| Sequences | 32 | YYFGSSPNWYFDV |
| | 33 | GSSPNWAMDY |
| | 34 | YFFGSSPNWAMDY |
| | 35 | YFFGSTPNWYFDV |
| | 36 | GWWGMDV |
| LCDR1 | 37 | RASQNINNYLH |
| Sequences | 38 | GATENIYGALN |
| | 39 | RASESIYGALN |
| | 40 | GASENIYGALH |
| | 41 | RASEGIYGALA |
| LCDR2 | 42 | AASNLQG |
| Sequences | 43 | GASNLAD |
| | 44 | GASNRYT |
| | 45 | DASNLES |
| | 46 | GASTRES |
| LCDR3 | 47 | LQTHAYPLT |
| Sequences | 48 | QNVLNTPLS |
| | 49 | QQVLNTPVS |
| | 50 | QNVLSTPWT |
| | 51 | QNVLNTPYT |
| | 52 | QQVLNTPLT |

TABLE 4
List of Exemplary Kabat CDR sequences

| Description | SEQ ID NO: | Amino Acid Sequence |
|---|---|---|
| HCDR1 | 54 | NYWMQ |
| Sequences | 55 | DFYMQ |
| | 56 | NYWIQ |
| | 57 | SYWIQ |
| | 58 | GNYMH |
| HCDR2 | 59 | EILPGTGSTEYAQKFQG |
| Sequences | 60 | EILPGTGHTEYAQKFQG |
| | 61 | EILAGSGSTEYSQKFRG |
| | 62 | EVLPGSGSTEYSQKFRG |
| | 63 | EILPGDGSTEYSQKFRG |
| | 64 | EILPTNGSTEYSQKFRG |
| | 65 | WINPKSGDTNYAQKFQG |
| HCDR3 | 66 | YFFGSTPNWYFDV |
| Sequences | 67 | YPFGSSPNWEFDY |
| | 68 | YYFGSSPNWYFDV |
| | 69 | YFFGSSPNWAMDY |
| | 70 | GW------WGMDV |
| LCDR1 | 71 | GATENIYGALN |
| Sequences | 72 | RASEGIYGALA |
| | 73 | RASESIYGALN |
| | 74 | GASENIYGALH |
| | 75 | RASQNINNYLH |
| LCDR2 | 76 | GASNLAD |
| Sequences | 77 | GASNRYT |
| | 78 | DASNLES |
| | 79 | GASTRES |
| | 80 | AASNLQG |
| LCDR3 | 81 | QNVLNTPLS |
| Sequences | 82 | QQVLNTPVS |
| | 83 | QQVLNTPLT |
| | 84 | QNVLSTPWT |
| | 85 | QNVLNTPYT |
| | 86 | LQTHAYPLT |

TABLE 5
Nucleotide sequences of heavy chain variable regions (VHs) and light chain variable
regions (VLs)of exemplary antibody molecules

| Antibody | Chain | Nucleotide Sequence | SEQ ID NO |
|---|---|---|---|
| ATG-001 | VH | caggtgcagctggtgcagagcggcgcggaagtgaaaaaaccgggcgcgagcgtgaaagtg | 100 |
| | | agctgcaaagcgagcggctatatttttaccaactattggatgcagtgggtgcgccaggcg | |
| | | ccgggccagggcctggaatggatgggcgaaattctgccgggcaccggcagcaccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 101 |
| | | attacctgcggcgcgaccgaaaacatttatggcgcgctgaactggtatcagcatgaaccg | |
| | | ggcaaagcgccgaaactgctgatttatggcgcgagcaacctggcggatggcgtgaccagc | |
| | | cgctttagcggcagcggcagcggcaccgattttaccctgaccattagcaccctgcagccg | |
| | | gaagattttgcgacctattattgccagaacgtgctgaacaccccgctgagctttggcggc | |
| | | ggcaccaaagtggatattaaa | |
| ATG-002 | VH | caggtgcagctggtgcagagcggcgcggaagtgaaaaaaccgggcgcgagcgtgaaagtg | 102 |
| | | agctgcaaagcgagcggctatatttttaccgatttttatatgcagtgggtgcgccaggcg | |
| | | ccgggccagggcctggaatggatgggcgaaattctgccgggcaccggcagcaccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 103 |
| | | attacctgcggcgcgaccgaaaacatttatggcgcgctgaactggtatcagcatgaaccg | |
| | | ggcaaagcgccgaaactgctgatttatggcgcgagcaacctggcggatggcgtgaccagc | |
| | | cgctttagcggcagcggcagcggcaccgattttaccctgaccattagcaccctgcagccg | |
| | | gaagattttgcgacctattattgccagaacgtgctgaacaccccgctgagctttggcggc | |
| | | ggcaccaaagtggatattaaa | |
| ATG-003 | VH | caggtgcagctggtgcagagcggcgcggaagtgaaaaaaccgggcgcgagcgtgaaagtg | 104 |
| | | agctgcaaagcgagcggccatatttttaccaactattggatgcagtgggtgcgccaggcg | |
| | | ccgggccagggcctggaatggatgggcgaaattctgccgggcaccggccataccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 105 |
| | | attacctgcggcgcgaccgaaaacatttatggcgcgctgaactggtatcagcatgaaccg | |
| | | ggcaaagcgccgaaactgctgatttatggcgcgagcaacctggcggatggcgtgaccagc | |
| | | cgctttagcggcagcggcagcggcaccgattttaccctgaccattagcaccctgcagccg | |
| | | gaagattttgcgacctattattgccagaacgtgctgaacaccccgctgagctttggcggc | |
| | | ggcaccaaagtggatattaaa | |
| ATG-004 | VH | caggtgcagctggtgcagagcggcgcggaagtgaaaaaaccgggcgcgagcgtgaaagtg | 106 |
| | | agctgcaaagcgagcggccatatttttaccgatttttatatgcagtgggtgcgccaggcg | |
| | | ccgggccagggcctggaatggatgggcgaaattctgccgggcaccggccataccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 107 |
| | | attacctgcggcgcgaccgaaaacatttatggcgcgctgaactggtatcagcatgaaccg | |
| | | ggcaaagcgccgaaactgctgatttatggcgcgagcaacctggcggatggcgtgaccagc | |
| | | cgctttagcggcagcggcagcggcaccgattttaccctgaccattagcaccctgcagccg | |
| | | gaagattttgcgacctattattgccagaacgtgctgaacaccccgctgagctttggcggc | |
| | | ggcaccaaagtggatattaaa | |
| ATG-005 | VH | caggtgcagctggtgcagagcggcgcggaagtgaaaaaaccgggcgcgagcgtgaaagtg | 108 |
| | | agctgcaaagcgagcggctatatttttaccaactattggatgcagtgggtgcgccaggcg | |
| | | ccgggccagggcctggaatggatgggcgaaattctgccgggcaccggcagcaccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 109 |
| | | attacctgcggcgcgaccgaaaacatttatggcgcgctgaactggtatcagcatgaaccg | |
| | | ggcaaagcgccgaaactgctgatttatggcgcgagcaaccgctataccggcgtgaccagc | |
| | | cgctttagcggcagcggcagcggcaccgattttaccctgaccattagcaccctgcagccg | |
| | | gaagattttgcgacctattattgccagcaggtgctgaacaccccggtgagctttggcggc | |
| | | ggcaccaaagtggatattaaa | |
| ATG-006 | VH | caggtgcagctggtgcagagcggcgcggaagtgaaaaaaccgggcgcgagcgtgaaagtg | 110 |
| | | agctgcaaagcgagcggctatatttttaccgatttttatatgcagtgggtgcgccaggcg | |
| | | ccgggccagggcctggaatggatgggcgaaattctgccgggcaccggcagcaccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 111 |
| | | attacctgcggcgcgaccgaaaacatttatggcgcgctgaactggtatcagcatgaaccg | |
| | | ggcaaagcgccgaaactgctgatttatggcgcgagcaaccgctataccggcgtgaccagc | |
| | | cgctttagcggcagcggcagcggcaccgattttaccctgaccattagcaccctgcagccg | |
| | | gaagattttgcgacctattattgccagcaggtgctgaacaccccggtgagctttggcggc | |
| | | ggcaccaaagtggatattaaa | |
| ATG-007 | VH | caggtgcagctggtgcagagcggcgcggaagtgaaaaaaccgggcgcgagcgtgaaagtg | 112 |
| | | agctgcaaagcgagcggccatatttttaccaactattggatgcagtgggtgcgccaggcg | |
| | | ccgggccagggcctggaatggatgggcgaaattctgccgggcaccggccataccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 113 |
| | | attacctgcggcgcgaccgaaaacatttatggcgcgctgaactggtatcagcatgaaccg | |
| | | ggcaaagcgccgaaactgctgatttatggcgcgagcaaccgctataccggcgtgaccagc | |
| | | cgctttagcggcagcggcagcggcaccgattttaccctgaccattagcaccctgcagccg | |
| | | gaagattttgcgacctattattgccagcaggtgctgaacaccccggtgagctttggcggc | |
| | | ggcaccaaagtggatattaaa | |
| ATG-008 | VH | caggtgcagctggtgcagagcggcgcggaagtgaaaaaaccgggcgcgagcgtgaaagtg | 114 |
| | | agctgcaaagcgagcggccatatttttaccgatttttatatgcagtgggtgcgccaggcg | |
| | | ccgggccagggcctggaatggatgggcgaaattctgccgggcaccggccataccgaatat | |
| | | gcgcagaaatttcagggccgcgtgaccatgacccgcgataccagcattagcaccgtgtat | |
| | | atggaagtgcgccgcctgcgcagcgatgataccgcggtgtattattgcgcgcgctatttt | |
| | | tttggcagcaccccgaactggtattttgatgtgtggggccagggcaccctggtgaccgtg | |
| | | agcagc | |
| | VL | gaaattgtgatgacccagagcccgagcagcctgagcgcgagcgtgggcgatcgcgtgacc | 115 |
| | | attacctgcggcgcgaccgaaaacatttatggcgcgctgaactggtatcagcatgaaccg | |
| | | ggcaaagcgccgaaactgctgatttatggcgcgagcaaccgctataccggcgtgaccagc | |
| | | cgctttagcggcagcggcagcggcaccgattttaccctgaccattagcaccctgcagccg | |
| | | gaagattttgcgacctattattgccagcaggtgctgaacaccccggtgagctttggcggc | |
| | | ggcaccaaagtggatattaaa | |

In an embodiment, the antibody molecule comprises one, two, or three CDRs of the VH region of an antibody molecule described herein, e.g., in Table 1 (e.g., any of monoclonal antibodies ATG-001, ATG-002, ATG-003, ATG-004, ATG-005, ATG-006, ATG-007, ATG-008, ATG-012, or ATG-013), using the Kabat or Chothia definitions of CDRs. In an embodiment, the antibody molecule comprises one, two, or three CDRs of the VL region of an antibody molecule described herein, e.g., in Table 1 (e.g., any of monoclonal antibodies ATG-001, ATG-002, ATG-003, ATG-004, ATG-005, ATG-006, ATG-007, ATG-008, ATG-012, or ATG-013), using the Kabat or Chothia definitions of CDRs. In an embodiment, the antibody molecule comprises one or more (e.g., two or three) CDRs of the VH region and/or one or more (e.g., two or three) CDRs of the VL region of an antibody molecule described herein, e.g., in Table 1 (e.g., any of monoclonal antibodies ATG-001, ATG-002, ATG-003, ATG-004, ATG-005, ATG-006, ATG-007, ATG-008, ATG-012, or ATG-013), using the Kabat or Chothia definitions of CDRs.

In an embodiment, the antibody molecule comprises one, two, or three VH CDRs described in Table 1. In an embodiment, the antibody molecule comprises one, two, or three VL CDRs described in Table 1. In an embodiment, the antibody molecule comprises one or more (e.g., two or three) VH CDRs and/or one or more (e.g., two or three) VL CDRs described in Table 1.

In an embodiment, the antibody molecule comprises one, two, three, or four frameworks of the VH region of an antibody molecule described in Table 1 (e.g., any of monoclonal antibodies ATG-001, ATG-002, ATG-003, ATG-004, ATG-005, ATG-006, ATG-007, ATG-008, ATG-012, or ATG-013). In an embodiment, the antibody molecule comprises one, two, three, or four frameworks of the VL region of an antibody molecule described in Table 1 (e.g., any of monoclonal antibodies ATG-001, ATG-002, ATG-003, ATG-004, ATG-005, ATG-006, ATG-007, ATG-008, ATG-012, or ATG-013). In an embodiment, the antibody molecule comprises one or more (e.g., two, three, or four) frameworks of the VH region and/or one or more (e.g., two, three, or four) frameworks of the VL region of an antibody molecule described in Table 1 (e.g., any of monoclonal antibodies ATG-001, ATG-002, ATG-003, ATG-004, ATG-005, ATG-006, ATG-007, ATG-008, ATG-012, or ATG-013).

In an embodiment, the antibody molecule comprises a heavy chain variable region of an antibody molecule described herein, e.g., in Table 1 (e.g., any of monoclonal antibodies ATG-001, ATG-002, ATG-003, ATG-004, ATG-005, ATG-006, ATG-007, ATG-008, ATG-012, or ATG-013). In an embodiment, the antibody molecule comprises a light chain variable region of an antibody molecule described herein, e.g., in Table 1 (e.g., any of monoclonal antibodies ATG-001, ATG-002, ATG-003, ATG-004, ATG-005, ATG-006, ATG-007, ATG-008, ATG-012, or ATG-013). In an embodiment, the antibody molecule comprises a heavy chain variable region and a light chain variable region of an antibody molecule described herein, e.g., in Table 1 (e.g., any of monoclonal antibodies ATG-001, ATG-002, ATG-003, ATG-004, ATG-005, ATG-006, ATG-007, ATG-008, ATG-012, or ATG-013).

In an embodiment, the antibody molecule comprises a heavy chain variable region having an amino acid sequence described in Table 1, or an amino acid sequence substantially identical thereof. In an embodiment, the antibody molecule comprises a light chain variable region having an amino acid sequence described in Table 1, or an amino acid sequence substantially identical thereof. In an embodiment, the antibody molecule comprises a heavy chain variable region having an amino acid sequence described in Table 1 (or an amino acid sequence substantially identical thereof) and a light chain variable region having an amino acid sequences described in Table 1 (or an amino acid sequence substantially identical thereof).

Exemplary VH and VL amino acid sequences are also described in Table 2. Exemplary Chothia and Kabat CDR amino acid sequences are also described in Tables 3-4, respectively.

In an embodiment, the antibody molecule comprises a heavy chain variable region encoded by a nucleotide sequence described in Table 5, or a nucleotide sequence substantially identical thereof. In an embodiment, the antibody molecule comprises a light chain variable region encoded by a nucleotide sequence described in Table 5, or a nucleotide sequence substantially identical thereof. In an embodiment, the antibody molecule comprises a heavy chain variable region encoded by a nucleotide sequence described in Table 5 (or a nucleotide sequence substantially identical thereof) and a light chain variable region encoded by a nucleotide sequence described in Table 5 (or a nucleotide sequence substantially identical thereof).

In an embodiment, the antibody molecule further comprises a heavy chain constant region. In an embodiment, the heavy chain constant region is an IgG1 constant region or a functional portion thereof. In another embodiment, the heavy chain constant region is an IgG2 constant region or a functional portion thereof. In an embodiment, the antibody molecule further comprises a light chain constant region. In an embodiment, the antibody molecule further comprises a heavy chain constant region. In an embodiment, the heavy chain constant region is an IgG3 constant region or a functional portion thereof. In an embodiment, the antibody molecule further comprises a heavy chain constant region. In an embodiment, the heavy chain constant region is an IgG4 constant region or a functional portion thereof. In an embodiment, the antibody molecule has a chimeric constant region comprising of IgG2, IgG3 and/or IgG4 isotypes. In an embodiment, the antibody molecule further comprises a heavy chain constant region and a light chain constant region. In an embodiment, the antibody molecule comprises a heavy chain constant region, a light chain constant region, and heavy and light chain variable regions of an antibody molecule described in Table 1. In an embodiment, the antibody molecule comprises a heavy chain constant region, a light chain constant region, and variable regions that comprise one, two, three, four, five, or six CDRs of an antibody molecule described in Table 1.

Exemplary heavy and light chain constant regions are described below.

>IgG2/4 heavy-constant (IgG2/4)
(SEQ ID NO: 116)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSQ
EDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVMHEALHNHYTQKSLSLSLGK
>IgG2/4 heavy-constant with Met-252-Tyr, Ser-254-
Thr and Thr-256-GLu substitutions (IgG2/4-YTE)
(SEQ ID NO: 117)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLYITREPEVTCVVVDVSQ
EDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVMHEALHNHYTQKSLSLSLGK
>IgG2/4 heavy-constant with Met-429-Leu and Asn-
435-Ser substitutions (IgG2/4-LS)
(SEQ ID NO: 118)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSQ
EDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVLHEALHSHYTQKSLSLSLGK
>IgG1 heavy-constant (IgG1)
(SEQ ID NO: 119)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
>light-constant
(SEQ ID NO: 120)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

In an embodiment, the antibody molecule comprises one or more (e.g., all) of the CDRs of ATG-004 or ATG-008 and a human IgG2/4 chimeric heavy chain constant region with Met-429-Leu and/or Asn-435-Ser substitutions as described herein. In an embodiment, the antibody molecule comprises one or more (e.g., all) of the CDRs of ATG-001, ATG-002, ATG-003, ATG-005, ATG-006, ATG-007, ATG-008, ATG-012, or ATG-013 and a human IgG1 constant region as described herein.

In some embodiments, the antibody molecule comprises a heavy chain variable region (VH) and a light chain variable region (VL), wherein the VH comprises three heavy chain complementarity determining regions (HCDR1, HCDR2, and HCDR3), wherein the VL comprises three light chain complementarity determining regions (LCDR1, LCDR2, and LCDR3),

wherein the VH comprises one, two, or all of the following:

(i) an HCDR1 comprising the amino acid sequence:

GX₁X₂FX₃X₄X₅Y,
wherein: X₁ is Y, F, or H;
   X₂ is I or T;
   X₃ is S or T;
   X₄ is N, S, D, or G; and
   X₅ is F, N, or absent
(SEQ ID NO: 87);

(ii) an HCDR2 comprising the amino acid sequence:

X₁X₂X₃X₄GX₅,
wherein: X₁ is L or N;
   X₂ is A or P;
   X₃ is G, T, or K;
   X₄ is S, D, N, T, or S; and
   X₅ is S, H, or D
(SEQ ID NO: 88);

(iii) an HCDR3 comprising the amino acid sequence:

X₁X₂X₃X₄X₅X₆X₇X₈X₉X₁₀X₁₁X₁₂X₁₃,
wherein: X₁ is Y or G;
   X₂ is P, Y, S, F, or W;
   X₃ is F, S, or W;
   X₄ is G or P;
   X₅ is S, N, or M;
   X₆ is 5, W, T, or D;
   X₇ is P, A, or V;
   X₈ is N, M, or absent;
   X₉ is W, D, or absent;
   X₁₀ is E, Y, A, or absent;
   X₁₁ is F, M, or absent;
   X₁₂ is D or absent; and
   X₁₃ is Y, V, or absent
(SEQ ID NO: 89); and

wherein the VL comprises one, two, or all of the following:

(iv) an LCDR1 comprising the amino acid sequence:

X₁AX₂X₃X₄IX₅X₆X₇LX₈,
wherein: X₁ is R or G;
   X₂ is S or T;
   X₃ is Q or E;
   X₄ is N, S, or G;
   X₅ is N or Y;
   X₆ is N or G;
   X₇ is Y or A; and
   X₈ is H, N, or A
(SEQ ID NO: 90);

(v) an LCDR2 comprising the amino acid sequence:

X₁ASX₂X₃X₄X₅,
wherein: X₁ is A, G, or D;
   X₂ is N or T;
   X₃ is L or R;
   X₄ is Q, A, Y, or E; or
   X₅ is G, D, T, or S
(SEQ ID NO: 91); and

(vi) an LCDR3 comprising the amino acid sequence:

X₁X₂X₃X₄X₅X₆PX₇X₈,
wherein: X₁ is L or Q;
   X₂ is Q or N;
   X₃ is T or V;
   X₄ is H or L;
   X₅ is A, N, or S;
   X₆ is Y or T;
   X₇ is L, V, W, or Y; or
   X₈ is T or S
(SEQ ID NO: 92).

In an embodiment, the VH comprises an HCDR1 comprising the amino acid sequence: GX₁X₂FX₃X₄X₅Y (SEQ ID NO: 87), which does not have one, two, three, four, or all of the following: X₁ is Y, X₂ is I, X₃ is 5, X₄ is S, or X₅ is N. In an embodiment, X₁ is not Y. In an embodiment, X₂ is not I. In an embodiment, X₃ is not S. In an embodiment, X₄ is not S. In an embodiment, X₅ is not N. In an embodiment, the HCDR1 does not comprise the amino acid sequence of SEQ ID NO: 16.

In an embodiment, the VH comprises an HCDR2 comprising the amino acid sequence: X₁X₂X₃X₄GX₅ (SEQ ID NO: 88), which does not have one, two, three, four, or all of the following: X₁ is L, X₂ is P, X₃ is G, X₄ is S, or X₅ is S. In an embodiment, X₁ is not L. In an embodiment, X₂ is not P. In an embodiment, X₃ is not G. In an embodiment, X₄ is not S. In an embodiment, X₅ is not S. In an embodiment, the HCDR1 does not comprise the amino acid sequence of SEQ ID NO: 56.

In an embodiment, the VH comprises an HCDR3 comprising the amino acid sequence: X₁X₂X₃X₄X₅X₆X₇X₈X₉X₁₀X₁₁X₁₂X₁₃ (SEQ ID NO: 89), which does not have one, two, three, four, five, six, seven, eight, nine, ten, eleven, twelve, or all of the following: X₁ is Y, X₂ is F, X₃ is F, X₄ is G, X₅ is 5, X₆ is 5, X₇ is P, X₈ is N, X₉ is W, X₁₀ is Y, X₁₁ is F, X₁₂ is D, or X₁₃ is V. In an embodiment, X₁ is not Y. In an embodiment X₂ is not F. In an embodiment X₃ is not F. In an embodiment X₄ is not G. In an embodiment X₅ is not S. In an embodiment X₆ is not S. In an embodiment X₇ is not P. In an embodiment X₈ is not N. In an embodiment X₉ is not W. In an embodiment X₁₀ is not Y. In an embodiment X₁₁ is not F. In an embodiment X₁₂ is not D. In an embodiment or X₁₃ is not V.

In an embodiment, the VL comprises an LCDR1 comprising the amino acid sequence: X₁AX₂X₃X₄IX₅X₆X₇LX₈ (SEQ ID NO: 90), which does not have one, two, three, four, five, six, seven, or all of the following: X₁ is G, X₂ is 5, X₃ is E, X₄ is N, X₅ is Y, X₆ is G, X₇ is A, or X₈ is N. In an embodiment, X₁ is not G. In an embodiment X₂ is not S. In an embodiment X₃ is not E. In an embodiment X₄ is not N. In an embodiment X₅ is not Y. In an embodiment X₆ is not G. In an embodiment X₇ is not A. In an embodiment or X₈ is not N.

In an embodiment, the VL comprises an LCDR2 comprising the amino acid sequence: X₁ASX₂X₃X₄X₅ (SEQ ID NO: 91), which does not have one, two, three, four, or all of the following: X₁ is G, X₂ is N, X₃ is L, X₄ is A, or X₅ is D. X₁ is not G, X₂ is not N, X₃ is not L, X₄ is not A, or X₅ is not D.

In an embodiment, the VL comprises an LCDR3 comprising the amino acid sequence: X₁X₂X₃X₄X₅X₆PX₇X₈ (SEQ ID NO: 92), which does not have one, two, three, four, five, six, seven, or all of the following: X₁ is Q, X₂ is N, X₃ is V, X₄ is L, X₅ is N, X₆ is T, X₇ is L, or X₈ is T. In an embodiment, X₁ is not Q. In an embodiment, X₂ is not N. In an embodiment, X₃ is not V. In an embodiment, X₄ is not L. In an embodiment, X₅ is not N. In an embodiment, X₆ is not T. In an embodiment, X₇ is not L. In an embodiment, X₈ is not T.

In embodiments, the antibody molecule comprises a VH and a VL, wherein the VH comprises three heavy chain complementarity determining regions (HCDR1, HCDR2, and HCDR3), wherein the VL comprises three light chain complementarity determining regions (LCDR1, LCDR2, and LCDR3),

wherein the VH comprises one, two, or all of the following:

(i) an HCDR1 comprising the amino acid sequence:

X₁X₂X₃X₄X₅
wherein: X₁ is N, D, S, or G;
   X₂ is Y, F, or N;
   X₃ is W or Y;
   X₄ is M or I; and
   X₅ is Q or H
(SEQ ID NO: 94);

(ii) an HCDR2 comprising the amino acid sequence:

X₁X₂X₃X₄X₅X₆GX₇TX₈YX₉QKFX₁₀G
wherein: X₁ is E or W;
   X₂ is I or V;
   X₃ is L or N;
   X₄ is P or A;
   X₅ is G, T, or K;
   X₆ is T, S, D, or N;
   X₇ is S, H, or D;
   X₈ is E or N;
   X₉ is A or S; and
   X₁₀ is Q or R
(SEQ ID NO: 95);
   (iii) an HCDR3 comprising the amino acid sequence:

X₁X₂X₃X₄X₅X₆X₇X₈WX₉X₁₀DX₁₁
wherein: X₁ is Y or G;
   X₂ is F, P, Y, or W;
   X₃ is F or absent;
   X₄ is G or absent;
   X₅ is S or absent;
   X₆ is T, S, or absent;
   X₇ is P or absent;
   X₈ is N or absent;
   X₉ is Y, E, A, or G;
   X₁₀ is F or M; and
   X₁₁ is V or Y
(SEQ ID NO: 96); and

wherein the VL comprises one, two, or all of the following:

(iv) an LCDR1 comprising the amino acid sequence:

X₁AX₂X₃X₄IX₅X₆X₇LX₈
wherein: X₁ is G or R;
   X₂ is T or S;
   X₃ is E or Q;
   X₄ is N, G, or S;
   X₅ is Y or N;
   X₆ is G or N;
   X₇ is A or Y; and
   X₈ is N, A, or H
(SEQ ID NO: 97);

(v) an LCDR2 comprising the amino acid sequence:

X₁ASX₂X₃X₄X₅
wherein: X₁ is G, D, or A;
   X₂ is N or T;
   X₃ is L or R;
   X₄ is A, Y, E, or Q; and
   X₅ is D, T, S, or G
(SEQ ID NO: 98); and

(vi) an LCDR3 comprising the amino acid sequence:

X₁X₂X₃X₄X₅X₆PX₇X₈
wherein: X₁ is Q or L;
   X₂ is N or Q;
   X₃ is V or T;
   X₄ is L or H;
   X₅ is N, S, or A;
   X₆ is T or Y;
   X₇ is L, V, W, or Y; and
   X₈ is S or T
(SEQ ID NO: 99).

In an embodiment, the VH comprises an HCDR1 comprising the amino acid sequence: X₁X₂X₃X₄X₅ (SEQ ID NO: 94), which does not have one, two, three, four, or all of the following: X₁ is N, X₂ is Y, X₃ is W, X₄ is I, or X₅ is Q. In an embodiment, X₁ is not N. In an embodiment, X₂ is not Y. In an embodiment, X₃ is not W. In an embodiment, X₄ is not I. In an embodiment, X₅ is not Q.

In an embodiment, the VH comprises an HCDR2 comprising the amino acid sequence: X₁X₂X₃X₄X₅X₆GX₇TX₈YX₉QKFX₁₀G (SEQ ID NO: 95), which does not have one, two, three, four, five, six, seven, or all of the following: X₁ is E, X₂ is I, X₃ is L, X₄ is P, X₅ is G, X₆ is 5, X₇ is S, or X₈ is E. In an embodiment, X₁ is not E. In an embodiment, X₂ is not I. In an embodiment, X₃ is not L. In an embodiment, X₄ is not P. In an embodiment, X₅ is not G. In an embodiment, X₆ is not S. In an embodiment, X₇ is not S. In an embodiment, or X₈ is not E.

In an embodiment, the VH comprises an HCDR3 comprising the amino acid sequence: X₁X₂X₃X₄X₅X₆X₇X₈WX₉X₁₀DX₁₁ (SEQ ID NO: 96), which does not have one, two, three, four, or all of the following: X₁ is Y, X₂ is F, X₃ is F, X₄ is G, X₅ is 5, X₆ is 5, X₇ is P, X₈ is N, X₉ is Y, X₁₀ is F, or X₁₁ is V. In an embodiment, X₁ is not Y. In an embodiment, X₂ is not F. In an embodiment, X₃ is not F. In an embodiment, X₄ is not G. In an embodiment, X₅ is not S. In an embodiment, X₆ is not S. In an embodiment, X₇ is not P. In an embodiment, X₈ is not N. In an embodiment, X₉ is not Y. In an embodiment, X₁₀ is not F. In an embodiment, or X₁₁ is not V.

In an embodiment, the VL comprises an LCDR1 comprising the amino acid sequence: X₁AX₂X₃X₄IX₅X₆X₇LX₈ (SEQ ID NO: 97), which does not have one, two, three, four, five, six, seven, or all of the following: X₁ is G, X₂ is 5, X₃ is E, X₄ is N, X₅ is Y, X₆ is G, X₇ is A, or X₈ is N. In an embodiment, X₁ is not Y. In an embodiment, X₂ is not F. In an embodiment, X₃ is not F. In an embodiment, X₄ is not G. In an embodiment, X₅ is not S. In an embodiment, X₆ is not S. In an embodiment, X₇ is not P. In an embodiment, X₈ is not N. In an embodiment, X₉ is not Y. In an embodiment, X₁₀ is not F. In an embodiment, or X₁₁ is not V.

In an embodiment, the VL comprises an LCDR2 comprising the amino acid sequence: X₁ASX₂X₃X₄X₅ (SEQ ID NO: 98), which does not have one, two, three, four, or all of the following: X₁ is G, X₂ is N, X₃ is L, X₄ is A, or X₅ is D. In an embodiment, X₁ is not G. In an embodiment, X₂ is not N. In an embodiment, X₃ is not L. In an embodiment, X₄ is not A. In an embodiment, or X₅ is not D.

In embodiments, the VL comprises an LCDR3 comprising the amino acid sequence: X₁X₂X₃X₄X₅X₆PX₇X₈ (SEQ ID NO: 99), which does not have one, two, three, four, five, six, seven, or all of the following: X₁ is Q, X₂ is N, X₃ is V, X₄ is L, X₅ is N, X₆ is T, X₇ is L, or X₈ is T. In an embodiment, X₁ is not Q. In an embodiment, X₂ is not N. In an embodiment, X₃ is not V. In an embodiment, X₄ is not L. In an embodiment, X₅ is not N. In an embodiment, X₆ is not T. In an embodiment, X₇ is not L. In an embodiment, or X₈ is not T.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 19; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 19; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 19; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 19; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises a VH comprising the amino acid sequence of SEQ ID NO: 1. In an embodiment, the antibody molecule comprises a VL comprising the amino acid sequence of SEQ ID NO: 10. In an embodiment, the antibody molecule comprises a VH comprising the amino acid sequence of SEQ ID NO: 1 and a VL comprising the amino acid sequence of SEQ ID NO: 10.

In an embodiment, the antibody molecule comprises a VH encoded by a nucleic acid comprising the nucleotide sequence of SEQ ID NO: 100. In an embodiment, the antibody molecule comprises a VL encoded by a nucleic acid comprising the nucleotide sequence of SEQ ID NO: 101. In an embodiment, the antibody molecule comprises a VH encoded by a nucleic acid comprising the nucleotide sequence of SEQ ID NO: 100 and a VL encoded by a nucleic acid comprising the nucleotide sequence of SEQ ID NO: 101.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 20; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 20; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 20; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 20; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises a VH comprising the amino acid sequence of SEQ ID NO: 2. In an embodiment, the antibody molecule comprises a VL comprising the amino acid sequence of SEQ ID NO: 10. In an embodiment, the antibody molecule comprises a VH comprising the amino acid sequence of SEQ ID NO: 2 and a VL comprising the amino acid sequence of SEQ ID NO: 10.

In an embodiment, the antibody molecule comprises a VH encoded by a nucleic acid comprising the nucleotide sequence of SEQ ID NO: 102. In an embodiment, the antibody molecule comprises a VL encoded by a nucleic acid comprising the nucleotide sequence of SEQ ID NO: 103. In an embodiment, the antibody molecule comprises a VH encoded by a nucleic acid comprising the nucleotide sequence of SEQ ID NO: 102 and a VL encoded by a nucleic acid comprising the nucleotide sequence of SEQ ID NO: 103.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 21; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 21; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 21; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 21; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises a VH comprising the amino acid sequence of SEQ ID NO: 3. In an embodiment, the antibody molecule comprises a VL comprising the amino acid sequence of SEQ ID NO: 10. In an embodiment, the antibody molecule comprises a VH comprising the amino acid sequence of SEQ ID NO: 3 and a VL comprising the amino acid sequence of SEQ ID NO: 10.

In an embodiment, the antibody molecule comprises a VH encoded by a nucleic acid comprising the nucleotide sequence of SEQ ID NO: 104. In an embodiment, the antibody molecule comprises a VL encoded by a nucleic acid comprising the nucleotide sequence of SEQ ID NO: 105. In an embodiment, the antibody molecule comprises a VH encoded by a nucleic acid comprising the nucleotide sequence of SEQ ID NO: 104 and a VL encoded by a nucleic acid comprising the nucleotide sequence of SEQ ID NO: 105.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 22; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48. In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 22; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 22; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 43; or an LCDR3 comprising an amino acid sequence that differs by no more than 1, 2, or 3 amino acid residues from, or has at least 85, 90, 95, 99 or 100% homology with, the amino acid sequence of SEQ ID NO: 48.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 22; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 43; or an LCDR3 comprising the amino acid sequence of SEQ ID NO: 48.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 76; or an LCDR3 comprising an amino acid sequence that differs by no more than 1, 2, or 3 amino acid residues from, or has at least 85, 90, 95, 99 or 100% homology with, the amino acid sequence of SEQ ID NO: 81.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 76; or an LCDR3 comprising the amino acid sequence of SEQ ID NO: 81.

In an embodiment, the antibody molecule comprises a VH comprising the amino acid sequence of SEQ ID NO: 4. In an embodiment, the antibody molecule comprises a VL comprising the amino acid sequence of SEQ ID NO: 10. In an embodiment, the antibody molecule comprises a VH comprising the amino acid sequence of SEQ ID NO: 4 and a VL comprising the amino acid sequence of SEQ ID NO: 10.

In an embodiment, the antibody molecule comprises a VH encoded by a nucleic acid comprising the nucleotide sequence of SEQ ID NO: 106. In an embodiment, the antibody molecule comprises a VL encoded by a nucleic acid comprising the nucleotide sequence of SEQ ID NO: 107. In an embodiment, the antibody molecule comprises a VH encoded by a nucleic acid comprising the nucleotide sequence of SEQ ID NO: 106 and a VL encoded by a nucleic acid comprising the nucleotide sequence of SEQ ID NO: 107.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 19; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 19; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 19; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 19; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises a VH comprising the amino acid sequence of SEQ ID NO: 1. In an embodiment, the antibody molecule comprises a VL comprising the amino acid sequence of SEQ ID NO: 11. In an embodiment, the antibody molecule comprises a VH comprising the amino acid sequence of SEQ ID NO: 1 and a VL comprising the amino acid sequence of SEQ ID NO: 11.

In an embodiment, the antibody molecule comprises a VH encoded by a nucleic acid comprising the nucleotide sequence of SEQ ID NO: 108. In an embodiment, the antibody molecule comprises a VL encoded by a nucleic acid comprising the nucleotide sequence of SEQ ID NO: 109. In an embodiment, the antibody molecule comprises a VH encoded by a nucleic acid comprising the nucleotide sequence of SEQ ID NO: 108 and a VL encoded by a nucleic acid comprising the nucleotide sequence of SEQ ID NO: 109.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 20; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 20; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 20; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 20; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises a VH comprising the amino acid sequence of SEQ ID NO: 2. In an embodiment, the antibody molecule comprises a VL comprising the amino acid sequence of SEQ ID NO: 11. In an embodiment, the antibody molecule comprises a VH comprising the amino acid sequence of SEQ ID NO: 2 and a VL comprising the amino acid sequence of SEQ ID NO: 11.

In an embodiment, the antibody molecule comprises a VH encoded by a nucleic acid comprising the nucleotide sequence of SEQ ID NO: 110. In an embodiment, the antibody molecule comprises a VL encoded by a nucleic acid comprising the nucleotide sequence of SEQ ID NO: 111. In an embodiment, the antibody molecule comprises a VH encoded by a nucleic acid comprising the nucleotide sequence of SEQ ID NO: 110 and a VL encoded by a nucleic acid comprising the nucleotide sequence of SEQ ID NO: 111.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 21; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 21; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 21; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 21; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 54; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 54; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises a VH comprising the amino acid sequence of SEQ ID NO: 3. In an embodiment, the antibody molecule comprises a VL comprising the amino acid sequence of SEQ ID NO: 11. In an embodiment, the antibody molecule comprises a VH comprising the amino acid sequence of SEQ ID NO: 3 and a VL comprising the amino acid sequence of SEQ ID NO: 11.

In an embodiment, the antibody molecule comprises a VH encoded by a nucleic acid comprising the nucleotide sequence of SEQ ID NO: 112. In an embodiment, the antibody molecule comprises a VL encoded by a nucleic acid comprising the nucleotide sequence of SEQ ID NO: 113. In an embodiment, the antibody molecule comprises a VH encoded by a nucleic acid comprising the nucleotide sequence of SEQ ID NO: 112 and a VL encoded by a nucleic acid comprising the nucleotide sequence of SEQ ID NO: 113.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 22; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 22; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 22; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 22; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 22; an HCDR2 comprising an amino acid sequence that differs by no more than 1, 2, or 3 amino acid residues from, or has at least 85, 90, 95, 99 or 100% homology with, the amino acid sequence of SEQ ID NO: 29; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 22; an HCDR2 comprising the amino acid sequence of SEQ ID NO: 29; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 60; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 60; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 20; an HCDR2 comprising an amino acid sequence that differs by no more than 1, 2, or 3 amino acid residues from, or has at least 85, 90, 95, 99 or 100% homology with, the amino acid sequence of SEQ ID NO: 28; or an HCDR3 comprising an amino acid sequence that differs by no more than 1, 2, or 3 amino acid residues from, or has at least 85, 90, 95, 99 or 100% homology with, the amino acid sequence of SEQ ID NO: 35, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 38; an LCDR2 comprising an amino acid sequence that differs by no more than 1, 2, or 3 amino acid residues from, or has at least 85, 90, 95, 99 or 100% homology with, the amino acid sequence of the SEQ ID NO: 44; or an LCDR3 comprising an amino acid sequence that differs by no more than 1, 2, or 3 amino acid residues from, or has at least 85, 90, 95, 99 or 100% homology with, the amino acid sequence of SEQ ID NO: 49.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 20; an HCDR2 comprising the amino acid sequence of SEQ ID NO: 28; and an HCDR3 comprising the amino acid sequence of SEQ ID NO: 35, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 38; an LCDR2 comprising the amino acid sequence of SEQ ID NO: 44; or an LCDR3 comprising the amino acid sequence of SEQ ID NO: 49.

In an embodiment, the antibody molecule comprises one or both of:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 55; an HCDR2 comprising an amino acid sequence that differs by no more than 1, 2, or 3 amino acid residues from, or has at least 85, 90, 95, 99 or 100% homology with, the amino acid sequence of SEQ ID NO: 59; or an HCDR3 comprising an amino acid sequence that differs by no more than 1, 2, or 3 amino acid residues from, or has at least 85, 90, 95, 99 or 100% homology with, the amino acid sequence of SEQ ID NO: 66, or

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 71; an LCDR2 comprising an amino acid sequence that differs by no more than 1, 2, or 3 amino acid residues from, or has at least 85, 90, 95, 99 or 100% homology with, the amino acid sequence of the SEQ ID NO: 77; or an LCDR3 comprising an amino acid sequence that differs by no more than 1, 2, or 3 amino acid residues from, or has at least 85, 90, 95, 99 or 100% homology with, the amino acid sequence of SEQ ID NO: 82.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 55; an HCDR2 comprising the amino acid sequence of SEQ ID NO: 59; and an HCDR3 comprising the amino acid sequence of SEQ ID NO: 66, and

(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 71; an LCDR2 comprising the amino acid sequence of SEQ ID NO: 77; or an LCDR3 comprising the amino acid sequence of SEQ ID NO: 82.

In an embodiment, the antibody molecule comprises a VH comprising the amino acid sequence of SEQ ID NO: 4. In an embodiment, the antibody molecule comprises a VL comprising the amino acid sequence of SEQ ID NO: 11. In an embodiment, the antibody molecule comprises a VH comprising the amino acid sequence of SEQ ID NO: 4 and a VL comprising the amino acid sequence of SEQ ID NO: 11.

In an embodiment, the antibody molecule comprises a VH encoded by a nucleic acid comprising the nucleotide sequence of SEQ ID NO: 114. In an embodiment, the antibody molecule comprises a VL encoded by a nucleic acid comprising the nucleotide sequence of SEQ ID NO: 115. In an embodiment, the antibody molecule comprises a VH encoded by a nucleic acid comprising the nucleotide sequence of SEQ ID NO: 114 and a VL encoded by a nucleic acid comprising the nucleotide sequence of SEQ ID NO: 115.

In an embodiment, the antibody molecule further comprises a heavy chain constant region, e.g., a heavy chain constant region described herein. In an embodiment, the antibody molecule further comprises a light chain constant region, e.g., a light chain constant region described herein. In an embodiment, the antibody molecule further comprises a heavy chain constant region, e.g., a heavy chain constant region described herein, and a light chain constant region, e.g., a light chain constant region described herein.

In an embodiment, the antibody molecule described herein has one or more (e.g., 2, 3, 4, 5, or all) of the following properties: specifically binds to C5 (e.g., human C5); prevents cleavage of C5, e.g., into C5a and C5b; prevents C5-based destruction of red blood cells; prevents chronic red blood cell destruction or hemolysis; reduces inflammation; or any combination thereof. In an embodiment, the antibody molecule comprises one or more (e.g., 2, 3, 4, 5, or all) CDRs, one or both of heavy chain variable region or light chain variable regions, or one or both of heavy chain or light chain, of any of antibody molecules ATG-001, ATG-002, ATG-003, ATG-004, ATG-005, ATG-006, ATG-007, ATG-008, ATG-012, or ATG-013. In an embodiment, the antibody molecule is suitable for use in treating a disorder in kidney, e.g., IgA nephropathy. In another embodiment, the antibody molecule is suitable for use in treating a disease or disorder, e.g., a complement-associated disorder, e.g., a complement-associated disorder described herein.

The antibody molecules described herein can have several advantageous properties. For example, the antibody molecules can be used to effectively treat, prevent or diagnose a disorder associated with C5, e.g., a disorder described herein, e.g., a complement-associated disorder, e.g., a complement-associated disorder described herein.

In an embodiment, the antibody molecule binds to C5, e.g., human C5, with high affinity, e.g., with a K_{D}′ of about 50 nM or less, e.g., about 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.5 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 10 nM, between 0.001 nM and 5 nM, between 0.001 nM and 2 nM, between 0.001 nM and 1 nM, between 0.001 nM and 0.5 nM, between 0.001 nM and 0.2 nM, between 0.001 nM and 0.1 nM, between 0.001 and 0.05 nM, between 0.001 and 0.02 nM, between 0.001 and 0.005 nM, between 5 nM and 10 nM, between 2 nM and 10 nM, between 1 nM and 10 nM, between 0.5 nM and 10 nM, between 0.2 nM and 10 nM, between 0.1 nM and 10 nM, between 0.05 nM and 10 nM, between 0.02 nM and 10 nM, between 0.01 nM and 10 nM, between 0.005 nM and 10 nM, between 0.002 and 10 nM, between 0.002 nM and 5 nM, between 0.005 nM and 2 nM, between 0.01 nM and 1 nM, between 0.02 nM and 0.5 nM, between 0.05 nM and 0.2 nM, between 0.001 nM and 0.002 nM, between 0.002 nM and 0.005 nM, between 0.005 nM and 0.01 nM, between 0.01 nM and 0.02 nM, between 0.02 nM and 0.05 nM, between 0.05 nM and 0.1 nM, between 0.1 nM and 0.2 nM, between 0.2 nM and 0.5 nM, between 0.5 nM and 1 nM, between 1 nM and 2 nM, between 2 nM and 5 nM, or between 5 nM and 10 nM.

In an embodiment, the antibody molecule binds to C5 with a K_{off} slower than 1×10⁻⁴, 5×10⁻⁵, or 1×10⁻⁵ s⁻¹. In an embodiment, the antibody molecule binds to C5 with a Kₒₙ faster than 1×10⁴, 5×10⁴, 1×10⁵, or 5×10⁵ M⁻¹s⁻¹.

In an embodiment, the antibody molecule binds to C5, e.g., human C5, with high affinity, e.g., with an EC₅₀ of about 2 μg/ml or less, e.g., about 1 μg/ml or less, 0.9 μg/ml or less, 0.8 μg/ml or less, 0.7 μg/ml or less, 0.6 μg/ml or less, 0.5 μg/ml or less, 0.4 μg/ml or less, 0.3 μg/ml or less, 0.2 μg/ml or less, 0.1 μg/ml or less, 0.09 μg/ml or less, 0.08 μg/ml or less, 0.07 μg/ml or less, 0.06 μg/ml or less, 0.05 μg/ml or less, 0.04 μg/ml or less, 0.03 μg/ml or less, 0.02 μg/ml or less, 0.01 μg/ml or less, 0.005 μg/ml or less, 0.002 μg/ml or less, 0.001 μg/ml or less, e.g., between 0.001 μg/ml and 2 μg/ml, e.g., between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 μg/ml and 0.05 μg/ml, between 0.001 μg/ml and 0.02 μg/ml, between 0.001 μg/ml and 0.01 μg/ml, between 0.001 μg/ml and 0.005 μg/ml, between 0.002 μg/ml and 1 μg/ml, between 0.005 μg/ml and 1 μg/ml, between 0.01 μg/ml and 1 μg/ml, between 0.02 μg/ml and 1 μg/ml, between 0.05 μg/ml and 1 μg/ml, between 0.1 μg/ml and 1 μg/ml, between 0.2 μg/ml and 1 μg/ml, between 0.5 μg/ml and 1 μg/ml, between 0.001 μg/ml and 1 μg/ml, between 0.002 μg/ml and 0.5 μg/ml, between 0.005 μg/ml and 0.2 μg/ml, between 0.01 μg/ml and 0.1 μg/ml, or between 0.02 μg/ml and 0.05 μg/ml, e.g., as determined by a method described herein.

In an embodiment, the antibody molecule reduces (e.g., inhibits, blocks, or neutralizes) one or more biological activities of C5 (e.g., human C5), e.g., at an IC₅₀ of about 50 μg/ml or less, e.g., about 20 μg/ml or less, 10 μg/ml or less, 9 μg/ml or less, 8 μg/ml or less, 7 μg/ml or less, 6 μg/ml or less, 5 μg/ml or less, 4 μg/ml or less, 3 μg/ml or less, 2 μg/ml or less, 1 μg/ml or less, 0.5 μg/ml or less, 0.2 μg/ml or less, 0.1 μg/ml or less, 0.05 μg/ml or less, 0.02 μg/ml or less, 0.01 μg/ml or less, 0.005 μg/ml or less, 0.002 μg/ml or less, or 0.001 μg/ml or less, e.g., between 0.001 μg/ml and 10 μg/ml, between 0.001 μg/ml and 5 μg/ml, between 0.001 μg/ml and 2 μg/ml, between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 and 0.05 μg/ml, between 0.001 and 0.02 μg/ml, between 0.001 and 0.005 μg/ml, between 5 μg/ml and 10 μg/ml, between 2 μg/ml and 10 μg/ml, between 1 μg/ml and 10 μg/ml, between 0.5 μg/ml and 10 μg/ml, between 0.2 μg/ml and 10 μg/ml, between 0.1 μg/ml and 10 μg/ml, between 0.05 μg/ml and 10 μg/ml, between 0.02 μg/ml and 10 μg/ml, between 0.01 μg/ml and 10 μg/ml, between 0.005 μg/ml and 10 μg/ml, between 0.002 and 10 μg/ml, between 0.002 μg/ml and 5 μg/ml, between 0.005 μg/ml and 2 μg/ml, between 0.01 μg/ml and 1 μg/ml, between 0.02 μg/ml and 0.5 μg/ml, between 0.05 μg/ml and 0.2 μg/ml, between 0.001 μg/ml and 0.002 μg/ml, between 0.002 μg/ml and 0.005 μg/ml, between 0.005 μg/ml and 0.01 μg/ml, between 0.01 μg/ml and 0.02 μg/ml, between 0.02 μg/ml and 0.05 μg/ml, between 0.05 μg/ml and 0.1 μg/ml, between 0.1 μg/ml and 0.2 μg/ml, between 0.2 μg/ml and 0.5 μg/ml, between 0.5 μg/ml and 1 μg/ml, between 1 μg/ml and 2 μg/ml, between 2 μg/ml and 5 μg/ml, or between 5 μg/ml and 10 μg/ml, e.g., as determined by a method described herein.

In an embodiment, the antibody molecule binds to a linear or conformational epitope on C5. In an embodiment, the antibody molecule binds, or substantially binds, to the same, similar, or overlapping epitope on C5, as a second antibody molecule (e.g., a monoclonal antibody described in Table 1). In an embodiment, the antibody molecule competes with a second antibody molecule (e.g., a monoclonal antibody described in Table 1) for binding to C5. In an embodiment, the epitope is a conformational epitope.

In an embodiment, LCDR1, LCDR2, LCDR3, HCDR1 and HCDR2 belong to Chothia CDR canonical classes 2, 1, 1, 1 and 2, respectively. In an embodiment, the antibody molecule comprises at least one of the paratope-paratope contacts described in Table 6.

TABLE 6
Exemplary paratope-paratope contacts in anti-C5 antibodies

| Amino acid-1 | Amino acid-2 |
|---|---|
| VH-30 (Thr) | VH-28 (Ile) |
| VH-31 (Asp) | VH-28 (Ile), VH-29 (Phe) |
| VH-32 (Phe) | VH-94 (Arg), VH-96 (Phe), VH-100 (Trp) |
| VH-94 (Arg) | VH-101 (Asp) |
| VL-25 (Ala) | VL-29 (Ile), VL-71 (Phe) |
| VL-55 (Tyr) | VL-47 (Leu), VL-58 (Val) |
| VL-90 (Gln) | VL-92 (Leu), VL-93 (Asn), VL-95 (Pro), VL-97 (Ser) |
| VL-96 (Val) | VL-98 (Phe) |

### Epitope

The antibody molecule described herein can bind to an epitope on C5 (e.g., human C5). For example, an epitope bound by an antibody molecule described herein can include one or more epitope contact points in a C5 protein sequence described herein.

### Animal Models

The antibody molecules described herein can be evaluated in vivo, e.g., using various animal models. For example, an animal model can be used to test the efficacy of an antibody molecule described herein in inhibiting C5 cleavage and/or in treating or preventing a disorder described herein, e.g., a complement-associated disorder, e.g., a complement-associated disorder described herein. Animal models can also be used, e.g., to investigate for side effects, measure concentrations of antibody molecules in situ, demonstrate correlations between a C5 function and a complement-associated disorder, e.g., a complement-associated disorder described herein.

Exemplary animal models for a complement-associated disorder, e.g., a complement-associated disorder described herein that can be used for evaluating an antibody molecule described herein include, but are not limited to, C5 deficient mice, e.g., reconstituted with human C5.

Exemplary animal models for other disorders described herein are also known in the art. Exemplary types of animals that can be used to evaluate the antibody molecules described herein include, but are not limited to, mice, rats, rabbits, guinea pigs, and monkeys.

### Pharmaceutical Compositions and Kits

In an aspect, this disclosure provides compositions, e.g., pharmaceutically acceptable compositions, which include an antibody molecule described herein (e.g., a humanized antibody molecule described herein), formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). In an embodiment, less than about 5%, e.g., less than about 4%, 3%, 2%, or 1% of the antibody molecules in the pharmaceutical composition are present as aggregates. In other embodiments, at least about 95%, e.g., at least about 96%, 97%, 98%, 98.5%, 99%, 99.5%, 99.8%, or more of the antibody molecules in the pharmaceutical composition are present as monomers. In an embodiment, the level of aggregates or monomers is determined by chromatography, e.g., high performance size exclusion chromatography (HP-SEC).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In an embodiment, the antibody molecule is administered by intravenous infusion or injection. In an embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules described herein can be administered by a variety of methods. Several are known in the art, and for many therapeutic, prophylactic, or diagnostic applications, an appropriate route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In an embodiment, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In an embodiment, an antibody molecule can be orally administered, for example, with an inert diluent or an assimilable edible carrier. The antibody molecule (and other ingredients, if desired) may also be enclosed in a hard or soft shell gelatin capsule, compressed into tablets, or incorporated directly into the subject's diet. For oral therapeutic administration, the antibody molecule may be incorporated with excipients and used in the form of ingestible tablets, buccal tablets, troches, capsules, elixirs, suspensions, syrups, wafers, and the like. To administer an antibody molecule by other than parenteral administration, it may be necessary to coat the compound with, or co-administer the compound with, a material to prevent its inactivation. Therapeutic, prophylactic, or diagnostic compositions can also be administered with medical devices, and several are known in the art.

Dosage regimens are adjusted to provide the desired response (e.g., a therapeutic, prophylactic, or diagnostic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the antibody molecule and the particular therapeutic, prophylactic, or diagnostic effect to be achieved, and (b) the limitations inherent in the art of compounding such an antibody molecule for the treatment of sensitivity in individuals.

An exemplary, non-limiting range for a therapeutically, prophylactically, or diagnostically effective amount of an antibody molecule is about 0.1-50 mg/kg body weight of a subject, e.g., about 0.1-30 mg/kg, e.g., about 1-30, 1-15, 1-10, 1-5, 5-10, or 1-3 mg/kg, e.g., about 1, 2, 3, 4, 5, 6, 7, 8, 9, 10, 15, 20, 30, 40, or 50 mg/kg. The antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², e.g., about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount,” “prophylactically effective amount,” or “diagnostically effectively amount” of an antibody molecule described herein.

A “therapeutically effective amount” refers to an amount effective, at dosages and for periods of time necessary, to achieve the desired therapeutic result. A therapeutically effective amount of the antibody molecule may vary according to factors such as the disease state, age, sex, and weight of the individual, and the ability of the antibody or antibody portion to elicit a desired response in the individual. A therapeutically effective amount is also one in which any toxic or detrimental effect of the antibody molecule is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody molecule to inhibit a measurable parameter can be evaluated in an animal model system predictive of efficacy in treating or preventing IgA nephropathy. Alternatively, this property of a composition can be evaluated by examining the ability of the antibody molecule to inhibit C5 cleavage, e.g., by an in vitro assay, e.g., by measuring C5b levels.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder, e.g., a disorder described herein, e.g., IgA nephropathy, can be diagnosed in vitro, ex vivo, or in vivo.

Also within this disclosure is a kit that comprises an antibody molecule, described herein. The kit can include one or more other elements including: instructions for use; other reagents, e.g., a label, a therapeutic agent, or an agent useful for chelating, or otherwise coupling, an antibody molecule to a label or therapeutic agent, or a radioprotective composition; devices or other materials for preparing the antibody molecule for administration; pharmaceutically acceptable carriers; and devices or other materials for administration to a subject.

### Nucleic Acids

The present disclosure also features nucleic acids comprising nucleotide sequences that encode the antibody molecules (e.g., heavy and light chain variable regions and CDRs of the antibody molecules), as described herein.

For example, the present disclosure features a first and second nucleic acid encoding heavy and light chain variable regions, respectively, of an antibody molecule chosen from one or more of the antibody molecules disclosed herein, e.g., an antibody molecule of Table 1, or a portion of an antibody molecule, e.g., the variable regions of Table 1. The nucleic acid can comprise a nucleotide sequence encoding any one of the amino acid sequences in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein).

In an embodiment, the nucleic acid can comprise a nucleotide sequence encoding at least one, two, or three CDRs from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, or three CDRs from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, three, four, five, or six CDRS from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In an embodiment, the nucleic acid can comprise a nucleotide sequence encoding at least one, two, or three CDRs from a heavy chain variable region having the nucleotide sequence as set forth in Table 5, a sequence substantially homologous thereto (e.g., a sequence at least about 85%, 90%, 95%, 99% or more identical thereto, and/or capable of hybridizing under the stringency conditions described herein). In an embodiment, the nucleic acid can comprise a nucleotide sequence encoding at least one, two, or three CDRs from a light chain variable region having the nucleotide sequence as set forth in Table 5, or a sequence substantially homologous thereto (e.g., a sequence at least about 85%, 90%, 95%, 99% or more identical thereto, and/or capable of hybridizing under the stringency conditions described herein). In an embodiment, the nucleic acid can comprise a nucleotide sequence encoding at least one, two, three, four, five, or six CDRS from heavy and light chain variable regions having the nucleotide sequence as set forth in Table 5, or a sequence substantially homologous thereto (e.g., a sequence at least about 85%, 90%, 95%, 99% or more identical thereto, and/or capable of hybridizing under the stringency conditions described herein).

In an embodiment, the nucleic acid comprises a nucleotide sequence as set forth in Table 5 or a sequence substantially homologous thereto (e.g., a sequence at least about 85%, 90%, 95%, 99% or more identical thereto, and/or capable of hybridizing under the stringency conditions described herein). In an embodiment, the nucleic acid comprises a portion of a nucleotide sequence as set forth in Table 5 or a sequence substantially homologous thereto (e.g., a sequence at least about 85%, 90%, 95%, 99% or more identical thereto, and/or capable of hybridizing under the stringency conditions described herein). The portion may encode, for example, a variable region (e.g., VH or VL); one, two, or three or more CDRs; or one, two, three, or four or more framework regions.

The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In an aspect, the application features host cells and vectors containing the nucleic acids described herein. The nucleic acids may be present in a single vector or separate vectors present in the same host cell or separate host cell, as described in more detail below.

### Vectors

Further provided herein are vectors that comprise nucleotide sequences encoding an antibody molecule described herein.

In an embodiment, the vector comprises a nucleotide encoding an antibody molecule described herein, e.g., as described in Table 1. In another embodiment, the vector comprises a nucleotide sequence described herein, e.g., in Table 5. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

The present disclosure also provides cells (e.g., host cells) comprising a nucleic acid encoding an antibody molecule as described herein. For example, the host cells may comprise a nucleic acid molecule having a nucleotide sequence described in Table 5, a sequence substantially homologous thereto (e.g., a sequence at least about 85%, 90%, 95%, 99% or more identical thereto, and/or capable of hybridizing under the stringency conditions described herein), or a portion of one of said nucleic acids. Additionally, the host cells may comprise a nucleic acid molecule encoding an amino acid sequence of Table 1, a sequence substantially homologous thereto (e.g., a sequence at least about 80%, 85%, 90%, 95%, 99% or more identical thereto), or a portion of one of said sequences.

In an embodiment, the host cells are genetically engineered to comprise nucleic acids encoding the antibody molecule described herein.

In an embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. In an embodiment, the cell (e.g., host cell) is an isolated cell.

### Uses of Antibody Molecules

The antibody molecules disclosed herein, as well as the pharmaceutical compositions disclosed herein, have in vitro, ex vivo, and in vivo therapeutic, prophylactic, and/or diagnostic utilities.

In an embodiment, the antibody molecule reduces (e.g., inhibits, blocks, or neutralizes) one or more biological activities of C5 (e.g., cleavage of C5). For example, these antibodies molecules can be administered to cells in culture, in vitro or ex vivo, or to a subject, e.g., a human subject, e.g., in vivo, to reduce (e.g., inhibits, blocks, or neutralizes) one or more biological activities of C5. In an embodiment, the antibody molecule inhibits, or substantially inhibit, cleavage of C5, e.g., human C5, e.g., to form C5a and C5b. Accordingly, in an aspect, the disclosure provides a method of treating, preventing, or diagnosing a disorder, e.g., a disorder described herein (e.g., IgA nephropathy), in a subject, comprising administering to the subject an antibody molecule described herein, such that the disorder is treated, prevented, or diagnosed. For example, the disclosure provides a method comprising contacting the antibody molecule described herein with cells in culture, e.g. in vitro or ex vivo, or administering the antibody molecule described herein to a subject, e.g., in vivo, to treat, prevent, or diagnose a disorder, e.g., a disorder associated with a complement-associated disorder, e.g., a complement-associated disorder described herein.

As used herein, the term “subject” is intended to include human and non-human animals. In an embodiment, the subject is a human subject, e.g., a human patient having a complement-associated disorder, e.g., a complement-associated disorder described herein, or at risk of having a complement-associated disorder, e.g., a complement-associated disorder described herein. The term “non-human animals” includes mammals and non-mammals, such as non-human primates. In an embodiment, the subject is a human. The methods and compositions described herein are suitable for treating human patients a complement-associated disorder, e.g., a complement-associated disorder described herein. Patients having a complement-associated disorder, e.g., a complement-associated disorder described herein, include those who have developed a complement-associated disorder, e.g., a complement-associated disorder described herein, but are (at least temporarily) asymptomatic, patients who have exhibited a symptom of a complement-associated disorder, e.g., a complement-associated disorder described herein, or patients having a disorder related to or associated with a complement-associated disorder, e.g., a complement-associated disorder described herein.

### Methods of Treating or Preventing Disorders

The antibody molecules described herein can be used to treat or prevent complement-associated disorders or symptoms thereof.

In an embodiment, the disorder is associated with aberrant levels of C5. In an embodiment, the antibody molecule is used to treat a subject having a disorder described herein, or is at risk of developing a disorder described herein.

Exemplary complement-associated disorders include, but are not limited to, AP-associated disorders and/or CP-associated disorders. Such disorders include, without limitation, rheumatoid arthritis (RA); antiphospholipid antibody syndrome; lupus nephritis; ischemia-reperfusion injury; atypical hemolytic uremic syndrome (aHUS); typical or infectious hemolytic uremic syndrome (tHUS); dense deposit disease (DDD); paroxysmal nocturnal hemoglobinuria (PNH); neuromyelitis optica (NMO); multifocal motor neuropathy (MMN); multiple sclerosis (MS); macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; Myasthenia Gravis; and traumatic brain injury.

In an embodiment, the complement-associated disorder is a complement-associated vascular disorder such as, but not limited to, a diabetes-associated vascular disorder (e.g., of the eye), central retinal vein occlusion, a cardiovascular disorder, myocarditis, a cerebrovascular disorder, a peripheral (e.g., musculoskeletal) vascular disorder, a renovascular disorder, a mesenteric/enteric vascular disorder, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy, and percutaneous transluminal coronary angioplasty (PTCA).

Additional complement-associated disorders include, without limitation, myasthenia gravis, cold agglutinin disease, dermatomyositis, Graves' disease, atherosclerosis, Alzheimer's disease, Guillain-Barré Syndrome, Degos' disease, graft rejection (e.g., transplant rejection), sepsis, burn (e.g., severe burn), systemic inflammatory response sepsis, septic shock, spinal cord injury, glomerulonephritis, Hashimoto's thyroiditis, type I diabetes, psoriasis, pemphigus, autoimmune hemolytic anemia (AIHA), idiopathic thrombocytopenic purpura (ITP), Goodpasture syndrome, antiphospholipid syndrome (APS), and catastrophic APS (CAPS). In some embodiments, the high concentration antibody solutions described herein can be used in methods for treating thrombotic microangiopathy (TMA), e.g., TMA associated with a complement-associated disorder such as any of the complement-associated disorders described herein.

Complement-associated disorders also include complement-associated pulmonary disorders such as, but not limited to, asthma, bronchitis, a chronic obstructive pulmonary disease (COPD), an interstitial lung disease, α-1 anti-trypsin deficiency, emphysema, bronchiectasis, bronchiolitis obliterans, alveolitis, sarcoidosis, pulmonary fibrosis, and collagen vascular disorders.

In an embodiment, the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

The antibody molecules described herein are typically administered at a frequency that keeps a therapeutically effective level of antibody molecules in the patient's system until the patient recovers. For example, the antibody molecules may be administered at a frequency that achieves a serum concentration sufficient for at least about 1, 2, 5, 10, 20, 30, or 40 antibody molecules to bind each C5 molecule. In an embodiment, the antibody molecules are administered every 1, 2, 3, 4, 5, 6, or 7 days, every 1, 2, 3, 4, 5, or 6 weeks, or every 1, 2, 3, 4, 5, or 6 months.

Methods of administering various antibody molecules are known in the art and are described below. Suitable dosages of the antibody molecules used will depend on the age and weight of the subject and the particular drug used.

In an embodiment, the antibody molecule is administered to the subject (e.g., a human subject) intravenously. In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg. In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg. In an embodiment, the antibody molecule is administered once a week, twice a week, once every two weeks, once every three weeks, once every four weeks, once every eight weeks, once a month, once every two months, or once every three months. In an embodiment, the antibody molecule is administered between 0.5 mg/kg and 3 mg/kg or between 50 mg and 150 mg, once a week, twice a week, once every two weeks, or once every four weeks.

The antibody molecules can be used by themselves or conjugated to a second agent, e.g., a bacterial agent, toxin, or protein, e.g., a second anti-C5 antibody molecule. This method includes: administering the antibody molecule, alone or conjugated to a second agent, to a subject requiring such treatment. The antibody molecules can be used to deliver a variety of therapeutic agents, e.g., a toxin, or mixtures thereof.

### Inflammatory Disorders

In an embodiment, the complement-associated disorder is an inflammatory disorder, e.g., PNH or aHUS. In an embodiment, the antibody molecule is used to treat a symptom associated with an inflammatory disorder, e.g., PNH or aHUS, or a combination thereof.

Paroxysmal nocturnal hemoglobinuria (PNH) is generally characterized by destruction of red blood cells (hemolytic anemia), blood clots (thrombosis), and impaired bone marrow function (e.g., making insufficient quantities of blood components).

Atypical hemolytic uremic syndrome (aHUS) is generally associated with chronic, uncontrolled activation of the complement system. aHUS is typically characterized by systemic thrombotic microangiopathy (TMA), the formation of blood clots in small blood vessels throughout the body, which can lead to stroke, heart attack, kidney failure, and death.

### Combination Therapies

The antibody molecules can be used in combination with other therapies. For example, the combination therapy can include an antibody molecule co-formulated with, and/or co-administered with, one or more additional therapeutic agents, e.g., one or more additional therapeutic agents described herein. In other embodiments, the antibody molecules are administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder. In an embodiment, two or more treatments are delivered prophylactically, e.g., before the subject has the disorder or is diagnosed with the disorder. In another embodiment, the two or more treatments are delivered after the subject has developed or diagnosed with the disorder. In an embodiment, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In an embodiment of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In an embodiment, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In an embodiment, the additional agent is a second antibody molecule, e.g., an antibody molecule different from a first antibody molecule. Exemplary antibody molecules that can be used in combination include, but are not limited to, any combination of the antibody molecules listed in Table 1.

In an embodiment, the antibody molecule is administered in combination with a second therapy to treat or prevent a complement-associated disorder, e.g., a complement-associated disorder described herein.

Exemplary therapies that can be used in combination with an antibody molecule or composition described herein to treat or prevent other disorders are also described in the section of “Methods of Treating or Preventing Disorders” herein.

### Methods of Diagnosis

In some aspects, the present disclosure provides a diagnostic method for detecting the presence of C5 (e.g., human C5) in vitro (e.g., in a biological sample, such as a biopsy or blood sample) or in vivo (e.g., in vivo imaging in a subject). The method includes: (i) contacting the sample with an antibody molecule described herein, or administering to the subject, the antibody molecule; (optionally) (ii) contacting a reference sample, e.g., a control sample (e.g., a control biological sample, such as a biopsy or blood sample) or a control subject with an antibody molecule described herein; and (iii) detecting formation of a complex between the antibody molecule and C5 in the sample or subject, or the control sample or subject, wherein a change, e.g., a statistically significant change, in the formation of the complex in the sample or subject relative to the control sample or subject is indicative of the presence of C5 in the sample. The antibody molecule can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting a polypeptide (e.g., C5) or a nucleic acid encoding the polypeptide includes, but is not limited to, cells, cell lysates, proteins or membrane extracts of cells, body fluids such as blood, or tissue samples such as biopsies.

Complex formation between the antibody molecule, and C5, can be detected by measuring or visualizing either the antibody molecule bound to C5 or unbound antibody molecule. Any suitable detection assays can be used, and conventional detection assays include an enzyme-linked immunosorbent assays (ELISA), a radioimmunoassay (RIA) or tissue immunohistochemistry. Alternative to labeling the antibody molecule, the presence of C5 can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of C5 in the sample is inversely proportional to the amount of labeled standard bound to the antibody molecule.

The antibody molecules described herein can be used to diagnose disorders that can be treated or prevented by the antibody molecules described herein. The detection or diagnostic methods described herein can be used in combination with other methods described herein to treat or prevent a disorder described herein.

The present disclosure also includes any of the following numbered paragraphs:

1. An isolated antibody molecule capable of binding to Complement component 5 (C5), comprising:

(a) a heavy chain variable region (VH) comprising an HCDR1 amino acid sequence of SEQ ID NO: 87, an HCDR2 amino acid sequence of SEQ ID NO: 88, and an HCDR3 amino acid sequence of SEQ ID NO: 89; and a light chain variable region (VL) comprising an LCDR1 amino acid sequence of SEQ ID NO: 90, an LCDR2 amino acid sequence of SEQ ID NO: 91, and an LCDR3 amino acid sequence of SEQ ID NO: 92; or

(b) a VH comprising an HCDR1 amino acid sequence of SEQ ID NO: 94, an HCDR2 amino acid sequence of SEQ ID NO: 95, and an HCDR3 amino acid sequence of SEQ ID NO: 96; and a light chain variable region (VL) comprising an LCDR1 amino acid sequence of SEQ ID NO: 97, an LCDR2 amino acid sequence of SEQ ID NO: 98, and an LCDR3 amino acid sequence of SEQ ID NO: 99.

2. The antibody molecule of paragraph 1, which comprises a VH comprising an HCDR1 amino acid sequence of SEQ ID NO: 87, an HCDR2 amino acid sequence of SEQ ID NO: 88, and an HCDR3 amino acid sequence of SEQ ID NO: 89; and a light chain variable region (VL) comprising an LCDR1 amino acid sequence of SEQ ID NO: 90, an LCDR2 amino acid sequence of SEQ ID NO: 91, and an LCDR3 amino acid sequence of SEQ ID NO: 92.

3. The antibody molecule of paragraph 1, which comprises a VH comprising an HCDR1 amino acid sequence of SEQ ID NO: 94, an HCDR2 amino acid sequence of SEQ ID NO: 95, and an HCDR3 amino acid sequence of SEQ ID NO: 96; and a light chain variable region (VL) comprising an LCDR1 amino acid sequence of SEQ ID NO: 97, an LCDR2 amino acid sequence of SEQ ID NO: 98, and an LCDR3 amino acid sequence of SEQ ID NO: 99.

4. The antibody molecule of any of paragraphs 1-3, which comprises a VH comprising an HCDR1 amino acid sequence of any of SEQ ID NOs: 16-23; an HCDR2 amino acid sequence of any of SEQ ID NOs: 24-30; and an HCDR3 amino acid sequence of any of SEQ ID NOs: 31-36; and a VL comprising an LCDR1 amino acid sequence of any of SEQ ID NOs: 37-41, an LCDR2 amino acid sequence of any of SEQ ID NOs: 42-46, and an LCDR3 amino acid sequence of any of SEQ ID NOs: 47-52.

5. The antibody molecule of paragraph 4, which comprises a VH comprising an HCDR1 amino acid sequence of SEQ ID NO: 19; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

6. The antibody molecule of paragraph 4, which comprises a VH comprising an HCDR1 amino acid sequence of SEQ ID NO: 20; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

7. The antibody molecule of paragraph 4, which comprises a VH comprising an HCDR1 amino acid sequence of SEQ ID NO: 21; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

8. The antibody molecule of paragraph 4, which comprises a VH comprising an HCDR1 amino acid sequence of SEQ ID NO: 22; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

9. The antibody molecule of paragraph 4, which comprises a VH comprising an HCDR1 amino acid sequence of SEQ ID NO: 19; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

10. The antibody molecule of paragraph 4, which comprises a VH comprising an HCDR1 amino acid sequence of SEQ ID NO: 20; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

11. The antibody molecule of paragraph 4, which comprises a VH comprising an HCDR1 amino acid sequence of SEQ ID NO: 21; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

12. The antibody molecule of paragraph 4, which comprises a VH comprising an HCDR1 amino acid sequence of SEQ ID NO: 22; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

13. The antibody molecule of any of paragraphs 1-3, which comprises a VH comprising an HCDR1 amino acid sequence of any of SEQ ID NOs: 54-58; an HCDR2 amino acid sequence of any of SEQ ID NOs: 59-65; and an HCDR3 amino acid sequence of any of SEQ ID NOs: 66-70; and a VL comprising an LCDR1 amino acid sequence of any of SEQ ID NOs: 71-75, an LCDR2 amino acid sequence of any of SEQ ID NOs: 76-80, and an LCDR3 amino acid sequence of any of SEQ ID NOs: 81-86.

14. The antibody molecule of paragraph 13, which comprises a VH comprising an HCDR1 amino acid sequence of SEQ ID NO: 54; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

15. The antibody molecule of paragraph 13, which comprises a VH comprising an HCDR1 amino acid sequence of SEQ ID NO: 55; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

16. The antibody molecule of paragraph 13, which comprises a VH comprising an HCDR1 amino acid sequence of SEQ ID NO: 54; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

17. The antibody molecule of paragraph 13, which comprises a VH comprising an HCDR1 amino acid sequence of SEQ ID NO: 55; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

18. The antibody molecule of paragraph 13, which comprises a VH comprising an HCDR1 amino acid sequence of SEQ ID NO: 54; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

19. The antibody molecule of paragraph 13, which comprises a VH comprising an HCDR1 amino acid sequence of SEQ ID NO: 55; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

20. The antibody molecule of paragraph 13, which comprises a VH comprising an HCDR1 amino acid sequence of SEQ ID NO: 54; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

21. The antibody molecule of paragraph 13, which comprises a VH comprising an HCDR1 amino acid sequence of SEQ ID NO: 55; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

22. The antibody molecule of any of paragraphs 1-21, which comprises a VH comprising an amino acid sequence at least 85%, 90%, or 95% identical to any of SEQ ID NOs: 1-9.

23. The antibody molecule of paragraph 22, which comprises a VH comprising an amino acid sequence of any of SEQ ID NOs: 1-9.

24. The antibody molecule of any of paragraphs 1-21, which comprises a VL comprising an amino acid sequence at least 85%, 90%, or 95% identical to any of SEQ ID NOs: 10-15.

25. The antibody molecule of paragraph 24, which comprises a VL comprising an amino acid sequence of any of SEQ ID NOs: 10-15.

26. The antibody molecule of any of paragraphs 1-21, which comprises a VH comprising an amino acid sequence at least 85%, 90%, or 95% identical to any of SEQ ID NOs: 1-9 and a VL comprising an amino acid sequence at least 85%, 90%, or 95% identical to any of SEQ ID NOs: 10-15.

27. The antibody molecule of paragraph 26, which comprises a VH comprising an amino acid sequence of any of SEQ ID NOs: 1-9 and a VL comprising an amino acid sequence of any of SEQ ID NOs: 10-15.

28. The antibody molecule of any of paragraphs 1-27, which comprise a VH comprising an amino acid sequence of SEQ ID NO: 1 and a VL comprising an amino acid sequence of SEQ ID NO: 10.

29. The antibody molecule of any of paragraphs 1-27, which comprise a VH comprising an amino acid sequence of SEQ ID NO: 2 and a VL comprising an amino acid sequence of SEQ ID NO: 10.

30. The antibody molecule of any of paragraphs 1-27, which comprise a VH comprising an amino acid sequence of SEQ ID NO: 3 and a VL comprising an amino acid sequence of SEQ ID NO: 10.

31. The antibody molecule of any of paragraphs 1-27, which comprise a VH comprising an amino acid sequence of SEQ ID NO: 4 and a VL comprising an amino acid sequence of SEQ ID NO: 10.

32. The antibody molecule of any of paragraphs 1-27, which comprise a VH comprising an amino acid sequence of SEQ ID NO: 1 and a VL comprising an amino acid sequence of SEQ ID NO: 11.

33. The antibody molecule of any of paragraphs 1-27, which comprise a VH comprising an amino acid sequence of SEQ ID NO: 2 and a VL comprising an amino acid sequence of SEQ ID NO: 11.

34. The antibody molecule of any of paragraphs 1-27, which comprise a VH comprising an amino acid sequence of SEQ ID NO: 3 and a VL comprising an amino acid sequence of SEQ ID NO: 11.

35. The antibody molecule of any of paragraphs 1-27, which comprise a VH comprising an amino acid sequence of SEQ ID NO: 4 and a VL comprising an amino acid sequence of SEQ ID NO: 11.

36. The antibody molecule of any of the preceding paragraphs, which comprises an antigen-binding fragment.

37. The antibody molecule of paragraph 36, wherein the antigen-binding fragment comprises a Fab, F(ab′)2, Fv, or scFv.

38. The antibody molecule of any of the preceding paragraphs, which comprises a heavy chain constant region chosen from the heavy chain constant regions of IgG1, IgG2, IgG3, or IgG4, or a chimera of two or more isotypes (e.g. IgG2/4), and optionally, wherein the heavy chain constant region comprises one or more amino acid modifications in the hinge, CH2 or CH3 region, as seen in IgG2/4-LS or IgG2/4-YTE.

39. The antibody molecule of any of the preceding paragraphs, which comprises a light chain constant region chosen from the light chain constant regions of kappa or lambda.

40. The antibody molecule of any of the preceding paragraphs, which comprises a heavy chain constant region chosen from the heavy chain constant regions of IgG1, IgG2, IgG3, IgG4, or a chimera of two or more isotypes (e.g. IgG2 and IgG4), and a light chain constant region chosen from the light chain constant regions of kappa or lambda.

41. The antibody molecule of any of the preceding paragraphs, which comprises an Fc region.

42. The antibody molecule of paragraph 41, wherein the Fc region comprises one or both of Met-429-Leu and Asn-435-Ser substitutions at residues corresponding to methionine 428 and asparagine 434, respectively, each in EU numbering.

43. The antibody molecule of paragraph 41 or 42, wherein the Fc region comprises one, two or three of Met-252-Tyr, Ser-254-Thr and Thr-256-Glu substitutions at residues corresponding to methionine 252, serine 254 and threonine 256, respectively, each in EU numbering.

44. The antibody molecule of any of the preceding paragraphs, which comprises two VH and two VL.

45. The antibody molecule of any of the preceding paragraphs, wherein said antibody molecule is a humanized antibody molecule.

46. The antibody molecule of any of the preceding paragraphs, wherein said antibody molecule is a monoclonal antibody molecule.

47. The antibody molecule of any of the preceding paragraphs, wherein said antibody molecule is a synthetic antibody molecule.

48. The antibody molecule of any of the preceding paragraphs, wherein said antibody molecule is a monospecific antibody molecule.

49. The antibody molecule of any of the preceding paragraphs, which is a multispecific antibody molecule.

50. The antibody molecule of any of the preceding paragraphs, which is a bispecific antibody molecule.

51. The antibody molecule of any of the preceding paragraphs, wherein the C5 is a human C5.

52. The antibody molecule of any of the preceding paragraphs, which binds to human C5 with a K_{D}′ of less than 0.1 μg/ml or 0.6 nM.

53. The antibody molecule of any of the preceding paragraphs, which binds to human C5 with a K_{D}′ of between 0.03 and 0.08 μg/ml or between 0.2 nM and 0.53 nM.

54. The antibody molecule of any of the preceding paragraphs, which binds to human C5 at an EC₅₀ of less than 2 μg/ml (e.g., less than 0.5 μg/ml) as determined by ELISA.

55. The antibody molecule of any of the preceding paragraphs, which binds to human C5 at an EC₅₀ of between 0.01 μg/ml and 2 μg/ml (e.g., between 0.02 μg/ml and 0.2 μg/ml) as determined by ELISA.

56. The antibody molecule of any of the preceding paragraphs, which binds to human C5 at an EC₅₀ of between 0.03 μg/ml and 0.8 μg/ml (e.g., between 0.03 μg/ml and 0.16 μg/ml) as determined by ELISA.

57. The antibody molecule of any of the preceding paragraphs, which inhibits hemolysis at an IC50 of less than 10 μg/ml as determined by an in vitro hemolysis assay.

58. The antibody molecule of any of the preceding paragraphs, which inhibits hemolysis at an IC50 of between 0.5 μg/ml and 6 μg/ml (e.g., between 2 μg/ml and 6 μg/ml) as determined by an in vitro hemolysis assay.

59. The antibody molecule of any of the preceding paragraphs, which inhibits hemolysis at an IC50 of between 0.5 μg/ml and 3.3 μg/ml (e.g., between 2.3 μg/ml and 3.3 μg/ml) as determined by an in vitro hemolysis assay.

60. The antibody molecule of any of the preceding paragraphs, which binds to human C5 comprising the amino acid sequence of SEQ ID NO: 53.

61. The antibody molecule of any of the preceding paragraphs, wherein:

(1) LCDR1, LCDR2, LCDR3, HCDR1 and HCDR2 belong to Chothia CDR canonical classes 2, 1, 1, 1 and 2, respectively; and

(2) the antibody molecule comprises at least one of the paratope-paratope contacts described in Table 6.

62. An antibody molecule capable of binding to C5, comprising a VH comprising an HCDR1, an HCDR2, and an HCDR3, and a VL comprising an LCDR1, an LCDR2, and an LCDR3, wherein:

(1) LCDR1, LCDR2, LCDR3, HCDR1 and HCDR2 belong to Chothia CDR canonical classes 2, 1, 1, 1 and 2, respectively; and

(2) the antibody molecule comprises at least one of the paratope-paratope contacts described in Table 6.

63. An antibody molecule that competes for binding to C5 with an antibody molecule of any of the preceding paragraphs.

64. An antibody molecule that binds to the same or overlapping epitope as the epitope recognized by an antibody molecule of any of the preceding paragraphs.

65. A pharmaceutical composition comprising the isolated antibody molecule of any of the preceding paragraphs and a pharmaceutically acceptable carrier, excipient or stabilizer.

66. An isolated nucleic acid encoding the VH, VL, or both, of the antibody molecule of any of paragraphs 1-65.

67. An expression vector comprising the nucleic acid of paragraph 66.

68. A host cell comprising the nucleic acid of paragraph 66 or the vector of paragraph 67.

69. A method of producing an antibody molecule, comprising culturing the host cell of paragraph 68 under conditions suitable for gene expression.

70. A method of inhibiting C5, comprising contacting C5 with an antibody molecule of any of paragraphs 1-64, or a pharmaceutical composition of paragraph 65.

71. The method of paragraph 70, wherein the contacting step occurs in vitro, ex vivo, or in vivo.

72. A method of treating a disorder, comprising administering to a subject in need thereof an antibody molecule of any of paragraphs 1-64, or a pharmaceutical composition of paragraph 65, in an amount effective to treat the disorder.

73. The method of paragraph 72, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

74. The method of paragraph 72 or 73, wherein the antibody molecule is administered to the subject at a dose between 0.1 mg/kg and 50 mg/kg.

75. The method of any of paragraphs 72-74, further comprising administering a second therapeutic agent or modality.

76. The method of paragraph 75, wherein the second therapeutic agent or modality is administered before, during, or after the antibody molecule is administered.

77. A method of preventing a disorder, comprising administering to a subject in need thereof an antibody molecule of any of paragraphs 1-64, or a pharmaceutical composition of paragraph 65, in an amount effective to treat the disorder.

78. The method of paragraph 77, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

79. A method of detecting C5, comprising (i) contacting a sample or a subject with an antibody molecule of any of paragraphs 1-64 under conditions that allow interaction of the antibody molecule and C5 to occur, and (ii) detecting formation of a complex between the antibody molecule and the sample or subject.

80. The method of paragraph 79, further comprising contacting a reference sample or subject with an antibody molecule of any of paragraphs 1-64 under conditions that allow interaction of the antibody molecule and C5 to occur, and (ii) detecting formation of a complex between the antibody molecule and the sample or subject.

81. An antibody molecule of any of paragraphs 1-64, or a pharmaceutical composition of paragraph 66, for use in treating a disorder in a subject.

82. The antibody molecule, or pharmaceutical composition, for use of paragraph 84, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

83. Use of an antibody molecule of any of paragraphs 1-64, or a pharmaceutical composition of paragraph 66, in the manufacture of a medicament for treating a disorder in a subject.

84. The use of paragraph 83, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

### EXAMPLES

### Example 1: Anti-05 Monoclonal Antibody (mAb) Generation and Characterization

### Generation of Anti-C5 Monoclonal Antibodies

Complement protein converge to a common pathway that causes splitting or activation of C3 to make C3a or C3b, resulting in the formation of various bioactive molecules such as C5a and C5b.

A number of exemplary anti-C5 mAbs (referred to as ATG-001, ATG-002, ATG-003, ATG-004, ATG-005, ATG-006, ATG-007, ATG-008, ATG-012, or ATG-013) were generated.

### Functional Analysis of Anti-C5 Monoclonal Antibodies

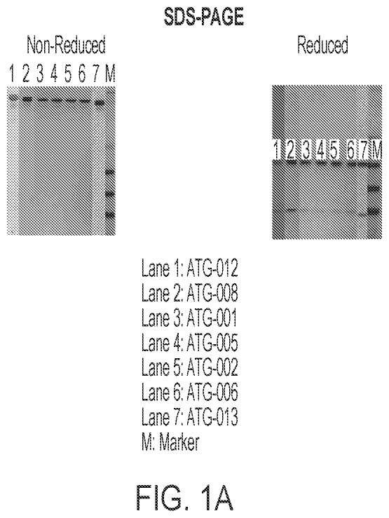

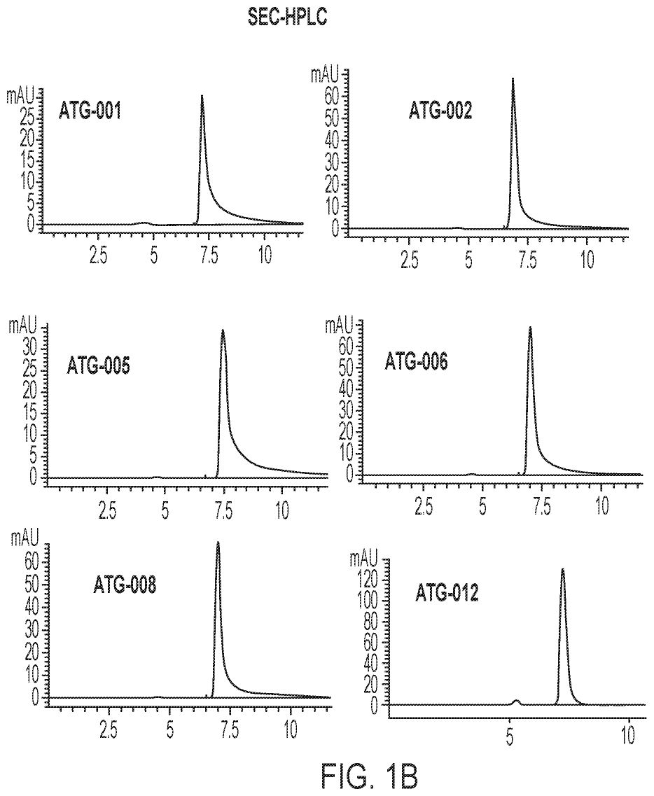

The exemplary anti-C5 mAbs were expressed in Expi CHO cells and analyzed for purity using SDS-PAGE under reducing and non-reducing conditions. Under reducing conditions, the mAbs migrated as two separate bands at 25 kDa and 50 kDa, matching the molecular weights of the light chain and heavy chain polypeptides, respectively. Under non-reducing conditions, the mAbs were observed as a single band at 150 kDa, which corresponded to the molecular weight of the intact antibody (FIG. 1A). Size exclusion chromatography (SEC) analysis of the mAbs revealed that the majority of the antibodies were in monomeric state (FIG. 1B).

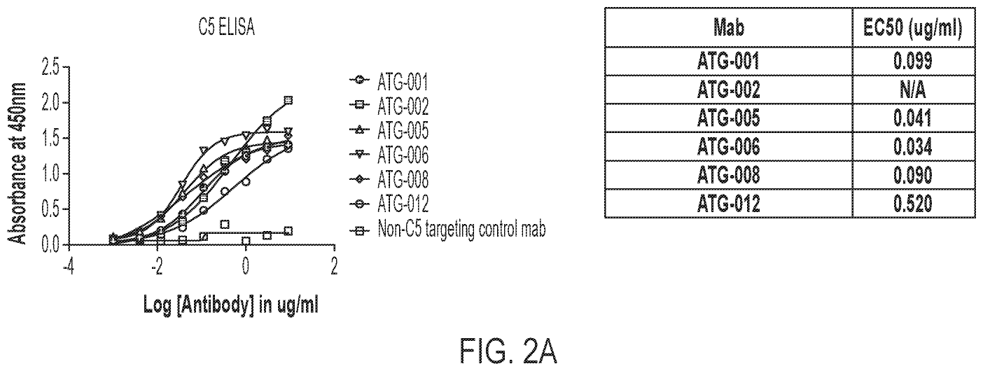

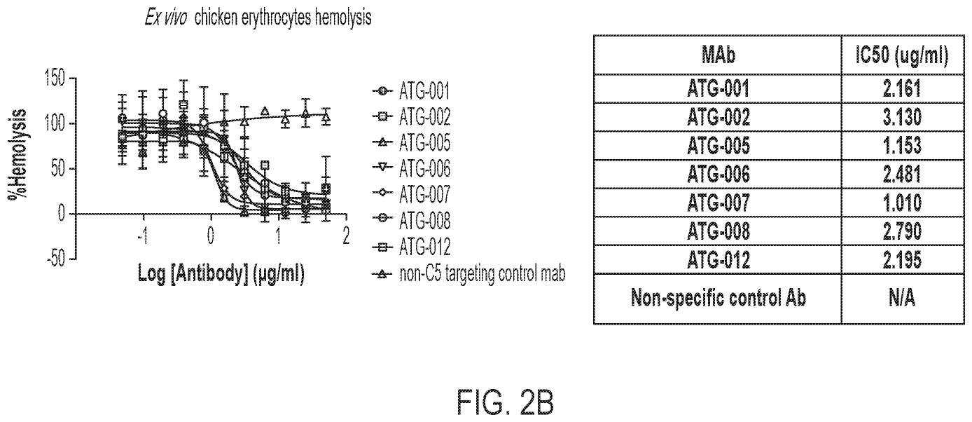

The exemplary antibodies were tested for binding to human C5 at pH 7.4 using ELISA. The EC50 values of the exemplary antibodies show that the exemplary antibodies exhibit strong binding affinity for human C5 (FIG. 2A). Additionally, the exemplary antibodies inhibited the hemolysis of chicken red blood cells in a dose-dependent manner with a half maximal inhibitory concentration (IC50) ranging between 0.5-6 ug/ml, showing that the exemplary antibodies suppressed C5 activity (FIG. 2B).

These data establish that the engineered antibodies exhibited anti-C5 activity in biochemical and in vitro studies.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. An isolated antibody molecule capable of binding to Complement component 5 (C5), comprising:
(a) a heavy chain variable region (VH) comprising an HCDR1 amino acid sequence of SEQ ID NO: 87, an HCDR2 amino acid sequence of SEQ ID NO: 88, and an HCDR3 amino acid sequence of SEQ ID NO: 89; and a light chain variable region (VL) comprising an LCDR1 amino acid sequence of SEQ ID NO: 90, an LCDR2 amino acid sequence of SEQ ID NO: 91, and an LCDR3 amino acid sequence of SEQ ID NO: 92; or
(b) a VH comprising an HCDR1 amino acid sequence of SEQ ID NO: 94, an HCDR2 amino acid sequence of SEQ ID NO: 95, and an HCDR3 amino acid sequence of SEQ ID NO: 96; and a light chain variable region (VL) comprising an LCDR1 amino acid sequence of SEQ ID NO: 97, an LCDR2 amino acid sequence of SEQ ID NO: 98, and an LCDR3 amino acid sequence of SEQ ID NO: 99.

2. The antibody molecule of claim 1, which comprises a VH comprising an HCDR1 amino acid sequence of SEQ ID NO: 87, an HCDR2 amino acid sequence of SEQ ID NO: 88, and an HCDR3 amino acid sequence of SEQ ID NO: 89; and a light chain variable region (VL) comprising an LCDR1 amino acid sequence of SEQ ID NO: 90, an LCDR2 amino acid sequence of SEQ ID NO: 91, and an LCDR3 amino acid sequence of SEQ ID NO: 92.

3. The antibody molecule of claim 1, which comprises a VH comprising an HCDR1 amino acid sequence of SEQ ID NO: 94, an HCDR2 amino acid sequence of SEQ ID NO: 95, and an HCDR3 amino acid sequence of SEQ ID NO: 96; and a light chain variable region (VL) comprising an LCDR1 amino acid sequence of SEQ ID NO: 97, an LCDR2 amino acid sequence of SEQ ID NO: 98, and an LCDR3 amino acid sequence of SEQ ID NO: 99.

4. The antibody molecule of any of claims 1-3, which comprises a VH comprising an HCDR1 amino acid sequence of any of SEQ ID NOs: 16-23; an HCDR2 amino acid sequence of any of SEQ ID NOs: 24-30; and an HCDR3 amino acid sequence of any of SEQ ID NOs: 31-36; and a VL comprising an LCDR1 amino acid sequence of any of SEQ ID NOs: 37-41, an LCDR2 amino acid sequence of any of SEQ ID NOs: 42-46, and an LCDR3 amino acid sequence of any of SEQ ID NOs: 47-52.

5. The antibody molecule of claim 4, which comprises a VH comprising an HCDR1 amino acid sequence of SEQ ID NO: 19; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

6. The antibody molecule of claim 4, which comprises a VH comprising an HCDR1 amino acid sequence of SEQ ID NO: 20; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

7. The antibody molecule of claim 4, which comprises a VH comprising an HCDR1 amino acid sequence of SEQ ID NO: 21; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

8. The antibody molecule of claim 4, which comprises a VH comprising an HCDR1 amino acid sequence of SEQ ID NO: 22; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 43, and an LCDR3 amino acid sequence of SEQ ID NO: 48.

9. The antibody molecule of claim 4, which comprises a VH comprising an HCDR1 amino acid sequence of SEQ ID NO: 19; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

10. The antibody molecule of claim 4, which comprises a VH comprising an HCDR1 amino acid sequence of SEQ ID NO: 20; an HCDR2 amino acid sequence of SEQ ID NO: 28; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

11. The antibody molecule of claim 4, which comprises a VH comprising an HCDR1 amino acid sequence of SEQ ID NO: 21; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

12. The antibody molecule of claim 4, which comprises a VH comprising an HCDR1 amino acid sequence of SEQ ID NO: 22; an HCDR2 amino acid sequence of SEQ ID NO: 29; and an HCDR3 amino acid sequence of SEQ ID NO: 35; and a VL comprising an LCDR1 amino acid sequence of SEQ ID NO: 38, an LCDR2 amino acid sequence of SEQ ID NO: 44, and an LCDR3 amino acid sequence of SEQ ID NO: 49.

13. The antibody molecule of any of claims 1-3, which comprises a VH comprising an HCDR1 amino acid sequence of any of SEQ ID NOs: 54-58; an HCDR2 amino acid sequence of any of SEQ ID NOs: 59-65; and an HCDR3 amino acid sequence of any of SEQ ID NOs: 66-70; and a VL comprising an LCDR1 amino acid sequence of any of SEQ ID NOs: 71-75, an LCDR2 amino acid sequence of any of SEQ ID NOs: 76-80, and an LCDR3 amino acid sequence of any of SEQ ID NOs: 81-86.

14. The antibody molecule of claim 13, which comprises a VH comprising an HCDR1 amino acid sequence of SEQ ID NO: 54; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

15. The antibody molecule of claim 13, which comprises a VH comprising an HCDR1 amino acid sequence of SEQ ID NO: 55; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

16. The antibody molecule of claim 13, which comprises a VH comprising an HCDR1 amino acid sequence of SEQ ID NO: 54; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

17. The antibody molecule of claim 13, which comprises a VH comprising an HCDR1 amino acid sequence of SEQ ID NO: 55; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 76, and an LCDR3 amino acid sequence of SEQ ID NO: 81.

18. The antibody molecule of claim 13, which comprises a VH comprising an HCDR1 amino acid sequence of SEQ ID NO: 54; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

19. The antibody molecule of claim 13, which comprises a VH comprising an HCDR1 amino acid sequence of SEQ ID NO: 55; an HCDR2 amino acid sequence of SEQ ID NO: 59; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

20. The antibody molecule of claim 13, which comprises a VH comprising an HCDR1 amino acid sequence of SEQ ID NO: 54; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

21. The antibody molecule of claim 13, which comprises a VH comprising an HCDR1 amino acid sequence of SEQ ID NO: 55; an HCDR2 amino acid sequence of SEQ ID NO: 60; and an HCDR3 amino acid sequence of SEQ ID NO: 66; and a VL comprising an LCDR1 amino acid sequence of SEQ ID NO: 71, an LCDR2 amino acid sequence of SEQ ID NO: 77, and an LCDR3 amino acid sequence of SEQ ID NO: 82.

22. The antibody molecule of any of claims 1-21, which comprises a VH comprising an amino acid sequence at least 85%, 90%, or 95% identical to any of SEQ ID NOs: 1-9.

23. The antibody molecule of claim 22, which comprises a VH comprising an amino acid sequence of any of SEQ ID NOs: 1-9.

24. The antibody molecule of any of claims 1-21, which comprises a VL comprising an amino acid sequence at least 85%, 90%, or 95% identical to any of SEQ ID NOs: 10-15.

25. The antibody molecule of claim 24, which comprises a VL comprising an amino acid sequence of any of SEQ ID NOs: 10-15.

26. The antibody molecule of any of claims 1-21, which comprises a VH comprising an amino acid sequence at least 85%, 90%, or 95% identical to any of SEQ ID NOs: 1-9 and a VL comprising an amino acid sequence at least 85%, 90%, or 95% identical to any of SEQ ID NOs: 10-15.

27. The antibody molecule of claim 26, which comprises a VH comprising an amino acid sequence of any of SEQ ID NOs: 1-9 and a VL comprising an amino acid sequence of any of SEQ ID NOs: 10-15.

28. The antibody molecule of any of claims 1-27, which comprise a VH comprising an amino acid sequence of SEQ ID NO: 1 and a VL comprising an amino acid sequence of SEQ ID NO: 10.

29. The antibody molecule of any of claims 1-27, which comprise a VH comprising an amino acid sequence of SEQ ID NO: 2 and a VL comprising an amino acid sequence of SEQ ID NO: 10.

30. The antibody molecule of any of claims 1-27, which comprise a VH comprising an amino acid sequence of SEQ ID NO: 3 and a VL comprising an amino acid sequence of SEQ ID NO: 10.

31. The antibody molecule of any of claims 1-27, which comprise a VH comprising an amino acid sequence of SEQ ID NO: 4 and a VL comprising an amino acid sequence of SEQ ID NO: 10.

32. The antibody molecule of any of claims 1-27, which comprise a VH comprising an amino acid sequence of SEQ ID NO: 1 and a VL comprising an amino acid sequence of SEQ ID NO: 11.

33. The antibody molecule of any of claims 1-27, which comprise a VH comprising an amino acid sequence of SEQ ID NO: 2 and a VL comprising an amino acid sequence of SEQ ID NO: 11.

34. The antibody molecule of any of claims 1-27, which comprise a VH comprising an amino acid sequence of SEQ ID NO: 3 and a VL comprising an amino acid sequence of SEQ ID NO: 11.

35. The antibody molecule of any of claims 1-27, which comprise a VH comprising an amino acid sequence of SEQ ID NO: 4 and a VL comprising an amino acid sequence of SEQ ID NO: 11.

36. The antibody molecule of any of the preceding claims, which comprises an antigen-binding fragment.

37. The antibody molecule of claim 36, wherein the antigen-binding fragment comprises a Fab, F(ab′)2, Fv, or scFv.

38. The antibody molecule of any of the preceding claims, which comprises a heavy chain constant region chosen from the heavy chain constant regions of IgG1, IgG2, IgG3, or IgG4, or a chimera of two or more isotypes (e.g. IgG2/4), and optionally, wherein the heavy chain constant region comprises one or more amino acid modifications in the hinge, CH2 or CH3 region, as seen in IgG2/4-LS or IgG2/4-YTE.

39. The antibody molecule of any of the preceding claims, which comprises a light chain constant region chosen from the light chain constant regions of kappa or lambda.

40. The antibody molecule of any of the preceding claims, which comprises a heavy chain constant region chosen from the heavy chain constant regions of IgG1, IgG2, IgG3, IgG4, or a chimera of two or more isotypes (e.g. IgG2 and IgG4), and a light chain constant region chosen from the light chain constant regions of kappa or lambda.

41. The antibody molecule of any of the preceding claims, which comprises an Fc region.

42. The antibody molecule of claim 41, wherein the Fc region comprises one or both of Met-429-Leu and Asn-435-Ser substitutions at residues corresponding to methionine 428 and asparagine 434, respectively, each in EU numbering.

43. The antibody molecule of claim 41 or 42, wherein the Fc region comprises one, two or three of Met-252-Tyr, Ser-254-Thr and Thr-256-Glu substitutions at residues corresponding to methionine 252, serine 254 and threonine 256, respectively, each in EU numbering.

44. The antibody molecule of any of the preceding claims, which comprises two VH and two VL.

45. The antibody molecule of any of the preceding claims, wherein said antibody molecule is a humanized antibody molecule.

46. The antibody molecule of any of the preceding claims, wherein said antibody molecule is a monoclonal antibody molecule.

47. The antibody molecule of any of the preceding claims, wherein said antibody molecule is a synthetic antibody molecule.

48. The antibody molecule of any of the preceding claims, wherein said antibody molecule is a monospecific antibody molecule.

49. The antibody molecule of any of the preceding claims, which is a multispecific antibody molecule.

50. The antibody molecule of any of the preceding claims, which is a bispecific antibody molecule.

51. The antibody molecule of any of the preceding claims, wherein the C5 is a human C5.

52. The antibody molecule of any of the preceding claims, which binds to human C5 with a K_{D}′ of less than 0.1 μg/ml or 0.6 nM.

53. The antibody molecule of any of the preceding claims, which binds to human C5 with a K_{D}′ of between 0.03 and 0.08 μg/ml or between 0.2 nM and 0.53 nM.

54. The antibody molecule of any of the preceding claims, which binds to human C5 at an EC₅₀ of less than 2 μg/ml (e.g., less than 0.5 μg/ml) as determined by ELISA.

55. The antibody molecule of any of the preceding claims, which binds to human C5 at an EC₅₀ of between 0.01 μg/ml and 2 μg/ml (e.g., between 0.02 μg/ml and 0.2 μg/ml) as determined by ELISA.

56. The antibody molecule of any of the preceding claims, which binds to human C5 at an EC₅₀ of between 0.03 μg/ml and 0.8 μg/ml (e.g., between 0.03 μg/ml and 0.16 μg/ml) as determined by ELISA.

57. The antibody molecule of any of the preceding claims, which inhibits hemolysis at an IC50 of less than 10 μg/ml as determined by an in vitro hemolysis assay.

58. The antibody molecule of any of the preceding claims, which inhibits hemolysis at an IC50 of between 0.5 μg/ml and 6 μg/ml (e.g., between 2 μg/ml and 6 μg/ml) as determined by an in vitro hemolysis assay.

59. The antibody molecule of any of the preceding claims, which inhibits hemolysis at an IC50 of between 0.5 μg/ml and 3.3 μg/ml (e.g., between 2.3 μg/ml and 3.3 μg/ml) as determined by an in vitro hemolysis assay.

60. The antibody molecule of any of the preceding claims, which binds to human C5 comprising the amino acid sequence of SEQ ID NO: 53.

61. The antibody molecule of any of the preceding claims, wherein:
(1) LCDR1, LCDR2, LCDR3, HCDR1 and HCDR2 belong to Chothia CDR canonical classes 2, 1, 1, 1 and 2, respectively; and
(2) the antibody molecule comprises at least one of the paratope-paratope contacts described in Table 6.

62. An antibody molecule capable of binding to C5, comprising a VH comprising an HCDR1, an HCDR2, and an HCDR3, and a VL comprising an LCDR1, an LCDR2, and an LCDR3, wherein:
(1) LCDR1, LCDR2, LCDR3, HCDR1 and HCDR2 belong to Chothia CDR canonical classes 2, 1, 1, 1 and 2, respectively; and
(2) the antibody molecule comprises at least one of the paratope-paratope contacts described in Table 6.

63. An antibody molecule that competes for binding to C5 with an antibody molecule of any of the preceding claims.

64. An antibody molecule that binds to the same or overlapping epitope as the epitope recognized by an antibody molecule of any of the preceding claims.

65. A pharmaceutical composition comprising the isolated antibody molecule of any of the preceding claims and a pharmaceutically acceptable carrier, excipient or stabilizer.

66. An isolated nucleic acid encoding the VH, VL, or both, of the antibody molecule of any of claims 1-64.

67. An expression vector comprising the nucleic acid of claim 66.

68. A host cell comprising the nucleic acid of claim 66 or the vector of claim 67.

69. A method of producing an antibody molecule, comprising culturing the host cell of claim 68 under conditions suitable for gene expression.

70. A method of inhibiting C5, comprising contacting C5 with an antibody molecule of any of claims 1-64, or a pharmaceutical composition of claim 65.

71. The method of claim 70, wherein the contacting step occurs in vitro, ex vivo, or in vivo.

72. A method of treating a disorder, comprising administering to a subject in need thereof an antibody molecule of any of claims 1-64, or a pharmaceutical composition of claim 65, in an amount effective to treat the disorder.

73. The method of claim 72, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

74. The method of claim 72 or 73, wherein the antibody molecule is administered to the subject at a dose between 0.1 mg/kg and 50 mg/kg.

75. The method of any of claims 72-74, further comprising administering a second therapeutic agent or modality.

76. The method of claim 75, wherein the second therapeutic agent or modality is administered before, during, or after the antibody molecule is administered.

77. A method of preventing a disorder, comprising administering to a subject in need thereof an antibody molecule of any of claims 1-64, or a pharmaceutical composition of claim 65, in an amount effective to treat the disorder.

78. The method of claim 77, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

79. A method of detecting C5, comprising (i) contacting a sample or a subject with an antibody molecule of any of claims 1-64 under conditions that allow interaction of the antibody molecule and C5 to occur, and (ii) detecting formation of a complex between the antibody molecule and the sample or subject.

80. The method of claim 79, further comprising contacting a reference sample or subject with an antibody molecule of any of claims 1-64 under conditions that allow interaction of the antibody molecule and C5 to occur, and (ii) detecting formation of a complex between the antibody molecule and the sample or subject.

81. An antibody molecule of any of claims 1-64, or a pharmaceutical composition of claim 65, for use in treating a disorder in a subject.

82. The antibody molecule, or pharmaceutical composition, for use of claim 81, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).

83. Use of an antibody molecule of any of claims 1-64, or a pharmaceutical composition of claim 65, in the manufacture of a medicament for treating a disorder in a subject.

84. The use of claim 83, wherein the disorder is a complement-associated disorder, optionally, wherein the complement-associated disorder is chosen from ischemia-reperfusion injury, atypical hemolytic uremic syndrome (aHUS), typical or infectious hemolytic uremic syndrome (tHUS), dense deposit disease (DDD), paroxysmal nocturnal hemoglobinuria (PNH), neuromyelitis optica (NMO), macular degeneration, thrombotic thrombocytopenic purpura (TTP); myasthenia gravis, cold agglutinin disease, Guillain-Barre Syndrome, Degos' disease, graft rejection, sepsis, glomerulonephritis, or thrombotic microangiopathy (TMA).
